(19) United States
(12) Patent Application Publication
(10) Pub. No.: US 2008/0108641 A1
(43) Pub. Date: May 8, 2008

(54) Compounds for treating inflammatory disorders, demyelinating disdorders and cancers

(76) Inventor: Alfred M. Ajami, Brookline, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/893,375

(22) Filed: Aug. 15, 2007

Related U.S. Application Data
(63) Continuation-in-part of application No. PCT/US07/03135, filed on Feb. 7, 2007.
(60) Provisional application No. 60/835,153, filed on Aug. 2, 2006.
(60) Provisional application No. 60/771,909, filed on Feb. 8, 2006.
(60) Provisional application No. 60/963,144, filed on Aug. 2, 2007.
(60) Provisional application No. 60/963,120, filed on Aug. 2, 2007.

(51) Int. Cl. A61K 31/437; C07D 471/06; A61P 29/00; A61P 35/00; A61P 25/28; A61P 19/02

(52) U.S. Cl. 514/288; 546/66

(57) ABSTRACT

Compounds of formula (I) and a method of treating a patient suffering from an inflammatory and/or demyelinating disorders, comprising administering to said patient a therapeutically effective amount of a compound of formula (I) or a pharmaceutically acceptable salt thereof. 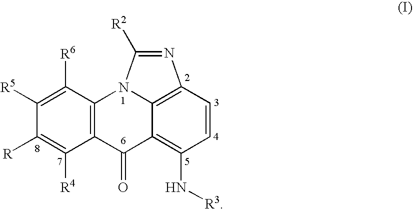 Definitions for the variables are provided therein.

### RELATED APPLICATIONS

This application is a continuation-in-part of International Application No. PCT/US2007/003135, which designated the United States and was filed on Feb. 7, 2007, published in English, which claims the benefit of U.S. Provisional Application No. 60/771,909, filed on Feb. 8, 2006 and U.S. Provisional Application No. 60/835,153, filed on Aug. 2, 2006.

This application claims the benefit of U.S. Provisional Application No. 60/963,144, filed on Aug. 2, 2007, and U.S. Provisional Application No. 60/963,120, filed on Aug. 2, 2007.

The entire teachings of the above applications are incorporated herein by reference.

### BACKGROUND OF THE INVENTION

There is a need for new pharmaceutically acceptable therapies for various types of cancer as well as inflammatory and demyelinating disorders.

### SUMMARY OF THE INVENTION

The present invention is directed to a class of novel compounds that can be used for treatment of certain cancers, immunocompromising, degenerative, inflammatory disorders and demyelinating diseases, including multiple sclerosis.

In one embodiment, the present invention is a compound of formula (I) or a pharmaceutically acceptable salt thereof: 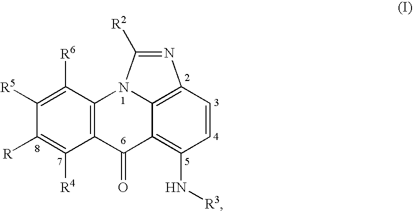
In formula (I):

R is R^{x}, a hydrolysable group, or alone or taken together with R⁴, or alternatively R⁵, and the intervening carbon atoms is a phenol isosteric group,
wherein R^{x} is —H, an optionally substituted alkyl, hydroxyl, alkoxy group, a halogen, or a group represented by the following structural formula: 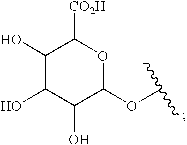
or, R and R⁵ taken together with their intervening carbon atoms form a 5, 6 or 7 member, optionally substituted, cycloalkyl or non-aromatic heterocycle; or R and R⁴ taken together with their intervening carbon atoms form a 5, 6 or 7 member, optionally substituted, cycloalkyl or non-aromatic heterocycle;

R² is —H, an optionally substituted C1-C10 alkyl or an optionally substituted aryl or aralkyl or heteroaryl;

R³ is —(CH₂)ₙ—NR^{a}R^{b}, wherein n is an integer from 1 to 5, and R^{a} and R^{b}, each independently are hydrogen or an optionally substituted alkyl, or R^{a} and R^{b}, taken together with the nitrogen to which they are attached, form group R^{y},
wherein R^{y} is a heteroaryl or a non-aromatic heterocycle, each optionally substituted at one or more substitutable carbon atoms with methyl, hydroxyl, or methoxy, and optionally N′-substituted with C1-C4 alkyl or C1-C4 alkyl substituted with —NR^{c}R^{d}, wherein R^{c} and R^{d} are individually H, methyl or ethyl; and

R⁴, R⁵ and R⁶, are each independently —H, —OH, a halogen or a C1-C6 alkoxy; or

R⁵ and R⁶ taken together with their intervening carbon atoms, form a 5, 6 or 7 member, optionally substituted cycloalkyl or non-aromatic heterocycle.

In one embodiment, when R is R^{x}, then R^{a} and R^{b} are not H or optionally substituted alkyl and R^{y} is not optionally substituted N-morpholinyl, optionally substituted N-piperazinyl, or optionally substituted N-pyrazinyl. In another embodiment, when R is R^{x}, then R^{a} and R^{b} are not H or optionally substituted alkyl and R^{y} is not optionally substituted N-morpholinyl, or optionally substituted N-pyrazinyl.

### BRIEF DESCRIPTION OF THE DRAWINGS

FIG. 1A is a bar plot illustrating inhibition of B-cells proliferation by Symadex™ following stimulation with lipopolysaccharide (LPS).

FIG. 1B is a bar plot illustrating inhibition of T-cells proliferation by Symadex™ following stimulation with concavanalin A (Con A).

FIG. 2A is a bar plot illustrating inhibition of IL-4 release by Symadex™ following stimulation with concavanalin A (Con A).

FIG. 2B is a bar plot illustrating inhibition of IL-10 release by Symadex™ following stimulation with concavanalin A (Con A).

FIG. 3 is a bar plot of the mean clinical score of the animals suffering from chronic stage Experimental Autoimmune Encephalomyelitis (EAE) at the indicated day post treatment with Symadex™ at 20 and 40 mg/kg versus vehicle control.

FIG. 4 is a time dependent chart showing mean clinical score (performance score) of the animals suffering from chronic stage Experimental Autoimmune Encephalomyelitis (EAE) at the indicated day post treatment with Symadex™ versus vehicle control.

FIG. 5 is a time dependent chart showing mean clinical score (performance score) of the animals suffering from chronic stage Experimental Autoimmune Encephalomyelitis (EAE) at the indicated day post treatment with Symadex™ after 4 weeks of dosing at 20 mg/kg versus vehicle control.

FIG. 6 is a time dependent chart showing mean clinical score (performance score) of the animals suffering from chronic stage Experimental Autoimmune Encephalomyelitis (EAE) at the indicated day post treatment with Symadex™ after 6 weeks of dosing at 20 mg/kg versus vehicle control.

FIG. 7 is a time dependent chart showing mean clinical score (performance score) of the animals suffering from chronic stage Experimental Autoimmune Encephalomyelitis (EAE) at the indicated day post treatment with Symadex™ after 8 weeks of dosing at 20 mg/kg versus vehicle control.

FIG. 8A is a bar chart showing the time course of T-cell counts of the animals suffering from chronic stage Experimental Autoimmune Encephalomyelitis (EAE) at the indicated day post treatment with Symadex™ after 4, 6 and 8 weeks of dosing at 20 mg/kg versus vehicle control.

FIG. 8B is a bar chart showing the time course of CD-4 cell counts of the animals suffering from chronic stage Experimental Autoimmune Encephalomyelitis (EAE) at the indicated day post treatment with Symadex™ after 4, 6 and 8 weeks of dosing at 20 mg/kg versus vehicle control.

FIG. 8C is a bar chart showing the time course of CD-8 cell counts of the animals suffering from chronic stage Experimental Autoimmune Encephalomyelitis (EAE) at the indicated day post treatment with Symadex™ after 4, 6 and 8 weeks of dosing at 20 mg/kg versus vehicle control.

FIG. 8D is a bar chart showing the time course of B-cell counts of the animals suffering from chronic stage Experimental Autoimmune Encephalomyelitis (EAE) at the indicated day post treatment with Symadex™ after 4, 6 and 8 weeks of dosing at 20 mg/kg versus vehicle control.

FIG. 9 is a time dependent chart showing mean clinical score (performance score) of the animals suffering from chronic stage Experimental Autoimmune Encephalomyelitis (EAE) at the indicated day post treatment with Symadex™ after 2 consecutive doses at 20 mg/kg given 72 hours apart versus vehicle control.

FIG. 10 is a time dependent chart showing mean clinical score of the animals suffering from acute stage Experimental Autoimmune Encephalomyelitis (EAE) at the indicated day post treatment with Symadex™ after 15 consecutive daily dose at 6 mg/kg.

FIG. 11 is a time dependent chart showing mean weight gain record of the animals suffering from acute stage Experimental Autoimmune Encephalomyelitis (EAE) at the indicated day post treatment with Symadex™ after 15 consecutive daily dose at 6 mg/kg.

FIG. 12 is a bar chart showing mean pathology scores on necropsy of the animals suffering from acute stage Experimental Autoimmune Encephalomyelitis (EAE) at the indicated day post treatment with Symadex™ after 15 consecutive daily dose at 6 mg/kg.

FIG. 13 is a time dependent chart bar chart showing mean performance of the animals suffering from acute stage Collagen Monoclonal Antibody (mAB) Induced Arthritis at the indicated day post treatment with Symadex™ after 3 consecutive daily oral doses at 30 mg/kg.

FIG. 14 is a list of chemical structures of pharmaceutical agents that can be co-administered with the compounds disclosed in the present invention.

### DETAILED DESCRIPTION OF THE INVENTION

It has now been discovered that administration of certain imidazoacridines and indazoles can be used to treat inflammatory diseases, diseases involving demyelination and certain cancers.

Compounds of the Present Invention

1. Imidazoacridines

Accordingly, in one embodiment, the present invention is a compound of formula (I) below or a pharmaceutically acceptable salt thereof. In another embodiment, the present invention is a method of treating an inflammatory or a demyelinating disease in a patient, comprising administering to said patient a therapeutically effective amount of a compound of formula (I) or a pharmaceutically acceptable salt thereof. 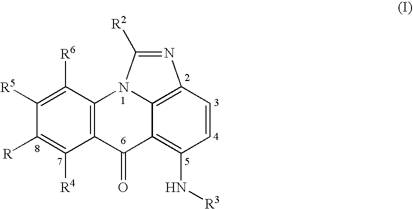
In formula (I), the substituents are each independently defined as follows.

R is R^{x}, a hydrolysable group as defined below, or alone or taken together with R⁴, or alternatively R⁵, and the intervening carbons is a phenol isosteric group as defined below.

R^{x} is —H, alkyl, hydroxyl, alkoxy group, a halogen, or a group represented by the following structural formula: 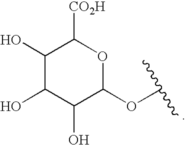
Included into the group R^{x} are such values of R that, when R and R⁵ are taken together with their intervening carbon atoms they form a 5, 6 or 7 member, optionally substituted, cycloalkyl or non-aromatic heterocycle, or such values of R that when R and R⁴ are taken together with their intervening carbon atoms, they form a 5, 6 or 7 member, optionally substituted, cycloalkyl or non-aromatic heterocycle.

R² is —H, an optionally substituted C1-C10 alkyl or an optionally substituted aryl or heteroaryl.

R³ is —(CH₂)ₙ—NR^{a}R^{b}, wherein n is an integer from 1 to 5, and R^{a} and R^{b}, each independently are hydrogen or an optionally substituted alkyl, or R^{a} and R^{b}, taken together with the nitrogen to which they are attached, form group R^{y}.

R^{y} is a heteroaryl or a non-aromatic heterocycle, each optionally substituted at one or more substitutable carbons with methyl, hydroxyl, or methoxy, and optionally N′-substituted with C1-C4 alkyl or C1-C4 alkyl substituted with —NR^{c}R^{d}, wherein R^{c} and R^{d} are individually H, methyl or ethyl.

In one embodiment, when R is R^{x}, then R^{a} and R^{b} are not H or optionally substituted alkyl and R^{y} is not optionally substituted N-morpholinyl, optionally substituted N-piperazinyl, or optionally substituted N-pyrazinyl.

In another embodiment, when R is R^{x}, then R^{a} and R^{b} are not H or optionally substituted alkyl and R^{y} is not optionally substituted N-morpholinyl, or optionally substituted N-pyrazinyl.

When R is R^{x} such that R and R⁴ or, alternatively, R and R⁵ are taken together with their intervening atoms, they preferably form a 5-6 membered cycloalkyl or 5-6 membered non-aromatic heterocycle containing one or two oxygen atoms and optionally substituted with methyl or hydroxyl. More preferably, when R is R^{x}, R⁴, R⁵ and R⁶ are each independently —H, —OH, a C1-C3 alkoxy or taken together with R, forms a methylenedioxy group; and R⁶ is —H, —OH, or a C1-C3 alkoxy. More preferably, R⁴, R⁵ and R⁶, are each independently, are independently each —H, —OH, or —OCH₃. The remainder of the variables take the values defined above in formula (I).

Preferably, R² is —H, optionally substituted C1-C8 alkyl, or phenyl, optionally substituted with one or more C1-C4 alkyl, C1-C4 alkoxy, C1-C4 haloalkoxy or cyano groups. More preferably, R²—H, methyl or ethyl. The remainder of the variables take the values defined above in formula (I).

In some embodiments of the compounds of formula (I), R^{y} is a heteroaryl, preferably N-pyrazinyl or N-pyridinyl. The remainder of the variables take the values defined above in formula (I).

Preferably, n is an integer from 1 to 5, and R^{a} and R^{b}, each independently are hydrogen or an alkyl, or R^{a} and R^{b}, taken together with the nitrogen to which they are attached, form a 5-7 membered non-aromatic heterocycle, optionally substituted at one or more substitutable carbons with methyl, hydroxyl, or methoxy, and optionally N′-substituted with C1-C4 alkyl or C1-C4 alkyl substituted with —NR^{c}R^{d}, wherein R^{c} and R^{d} are individually H, methyl or ethyl.

Preferably, n is 2 or 3 and R^{a} and R^{b}, is each independently a hydrogen or a C1-C3 alkyl. Alternatively, R^{a} and R^{b}, taken together with the nitrogen to which they are attached, form group R^{y}. The remainder of the variables take the values defined above in formula (I).

In another preferred embodiment, R^{y} is a 5-7 member non-aromatic heterocycle, optionally substituted at one or more substitutable carbons with methyl, hydroxyl, or methoxy, and optionally N′-substituted with C1-C4 alkyl or C1-C4 alkyl substituted with —NR^{c}R^{d}, wherein R^{c} and R^{d} are individually H, methyl or ethyl. Examples of R^{y} are listed below: 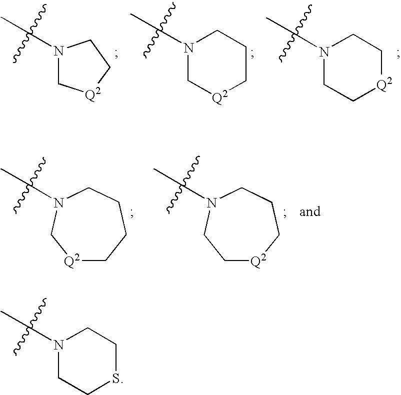
In these embodiments, Q² is CH₂, NH, or NR¹⁰², wherein R¹⁰² is methyl or ethyl.

In one embodiment, R is a hydrolysable group. The remainder of the variables take the values defined above in formula (I). As used herein, the term “hydrolysable group” means a derivatives of the compound of formula (I) at position 8, which yields the parent core molecule upon hydrolysis, either spontaneously under acidic or basic conditions in a physiological environment, e.g. blood, metabolically active tissues (liver, kidney, lung, brain) or catalyzed hydrolysis by enzyme, e.g. esterase, peptidases, hydrolases, oxidases, dehydrogenases, lyases or ligases.

Preferably, when R is a hydrolysable group, R is selected from groups represented by structural formulas (II)-(VII):

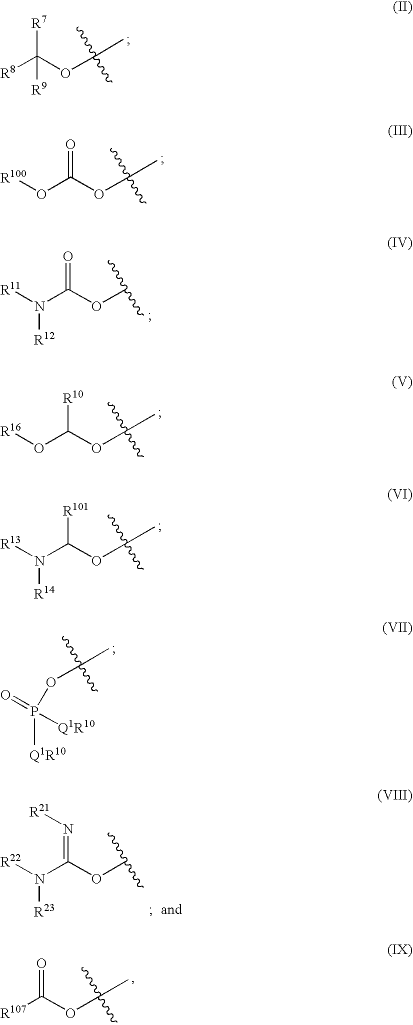

In variables in structural formulas (II)-(IX) are defined in the paragraphs below:

R⁷ and R⁸ are independently each H, optionally substituted C1-C6 alkyl, optionally substituted aryl or aralkyl. Preferably, R⁷ and R⁸ are independently each H, optionally substituted C1-C6 alkyl, phenyl or benzyl, optionally substituted with one or more hydroxyl, C1-C3 alkoxy, amino, alkylamino, halogen, C1-C3 haloalkyl or C1-C3 haloalkoxy groups. More preferably, R⁷ and R⁸ are each independently H, methyl or ethyl. The remainder of the variables take the values defined above in formula (I).

R⁹ is carboxyl, carboxamide optionally N-substituted with C1-C4 alkyl, C1-C6 alkanoyl, C1-C6 carbalkoxy, or optionally substituted aroyl. Preferably, R⁹ is a C1-C4 alkanoyl. The remainder of the variables take the values defined above in formula (I).

Q¹ is O or NH and R¹⁰ is H, optionally substituted C1-C6 alkyl or optionally substituted aryl or aralkyl. Preferably, R¹⁰ is H, C1-C4 alkyl, phenyl or benzyl, optionally substituted with one or more hydroxyl, C1-C3 alkoxy, amino, alkylamino, halogen, haloalkyl or haloalkoxy groups. More preferably, R¹⁰ is a H or C1-C4 alkyl. The remainder of the variables take the values defined above in formula (I).

R¹¹ and R¹² are independently each H, optionally substituted C1-C6 alkyl or, taken together with the atom to which they are attached, form an optionally substituted non-aromatic heterocycle. Preferably, R¹¹ and R¹² are independently each a H, methyl or ethyl or, taken together with the atom to which they are attached form non-aromatic heterocycle, optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl. More preferably, NR¹¹R¹² is N-pyrrolidinyl, N-piperidinyl, N-morpholinyl, N-thiomorpholinyl or N-piperazinyl, optionally N′-substituted with C1-C4 alkyl or C1-C4 alkyl substituted with —NR^{c}R^{d}, wherein R^{c} and R^{d} are individually H, methyl or ethyl. The remainder of the variables take the values defined above in formula (I).

R¹³ and R¹⁴ are each independently H, optionally substituted C1-C6 alkyl, optionally substituted C1-C6 alkanoyl, or optionally substituted aroyl, or, taken together with the atom to which they are attached, form an optionally substituted heteroaryl or non-aromatic optionally substituted heterocycle. Preferably, groups R¹³ and R¹⁴ are selected so that group (VI) above is represented by structural formulas (VIa) or (VIb): 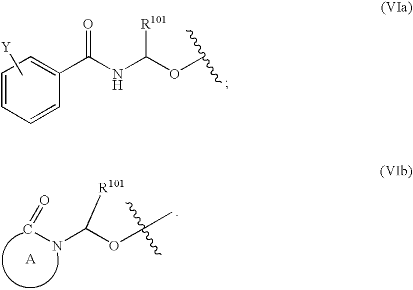
In structural formulas (VIa) and (VIb), Y is a halogen, —NO₂, —NH₂, —COOH, alkyl, C1-C3 carbalkoxy, C1-C3 alkoxy group, C1-C3 haloalkyl or C1-C3 haloalkoxy, and ring A is a 5-7 membered non-aromatic heterocycle optionally substituted at one or more substitutable ring carbon atoms with methyl, hydroxyl, oxo or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl. Examples of ring A include the following groups: 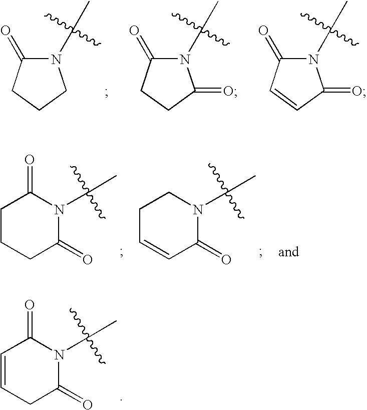
The remainder of the variables take the values defined above in formula (I).

R¹⁶ is optionally substituted C1-C6 alkyl, optionally substituted aryl or aralkyl, C1-C6 alkanoyl, or optionally substituted aroyl. Preferably, R¹⁶ is optionally substituted C1-C6 alkanoyl; more preferably, R¹⁶ is a branched C3-C6 alkanoyl. The remainder of the variables take the values defined above in formula (I).

R²¹ is optionally substituted C1-C10 alkyl, or an optionally substituted aryl or aralkyl or, R²¹ and R²² taken together with their intervening atoms form a 5-7 membered non-aromatic heterocycle. When R²² is not a part of a heterocycle defined above, the R²² and R²³ are each independently —H, or optionally substituted C1-C6 alkyl, provided that R²² and R²³ are not simultaneously hydrogens. Preferably, either R²¹ is optionally substituted C1-C10 alkyl, phenyl or benzyl optionally substituted with a halogen, —NO₂, —NH₂, —COOH, alkyl, C1-C3 carbalkoxy, C1-C3 alkoxy group, C1-C3 haloalkyl or C1-C3 haloalkoxy, and R²² and R²³ are each independently —H, or a C1-C3 alkyl; or R²¹ and R²², taken together with their intervening atoms, form a 5 or 6 membered non-aromatic heterocycle and R²³ is —H, or a C1-C3 alkyl. The remainder of the variables take the values defined above in formula (I).

R¹⁰⁰ is optionally substituted C1-C6 alkyl or optionally substituted aryl or aralkyl. In one embodiment, R¹⁰⁰ is phenyl or benzyl optionally substituted with a halogen, —NO₂, —NH₂, —COOH, alkyl, C1-C3 carbalkoxy, C1-C3 alkoxy group, C1-C3 haloalkyl or C1-C3 haloalkoxy. Preferably, R¹⁰⁰ is a C1-C4 alkyl. The remainder of the variables take the values defined above in formula (I).

R¹⁰¹ is H, optionally substituted C1-C6 alkyl or optionally substituted aryl or aralkyl. In one embodiment, R¹⁰⁰ is phenyl or benzyl optionally substituted with a halogen, —NO₂, —NH₂, —COOH, alkyl, C1-C3 carbalkoxy, C1-C3 alkoxy group, C1-C3 haloalkyl or C1-C3 haloalkoxy. Preferably, R¹⁰¹ is H or C1-C4 alkyl. More preferably, R¹⁰¹ is H, methyl or ethyl. The remainder of the variables take the values defined above in formula (I).

R¹⁰⁷ is C1-C6 alkyl, optionally substituted aryl or aralkyl, or a non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}. Preferably, R¹⁰⁷ is C1-C6 alkyl optionally substituted with —OH, —SH, halogen, cyano, nitro, amino, —COOH, a C1-C3 alkyl, C1-C3 haloalkyl, C1-C3 alkoxy, C1-C3 haloalkoxy or C1-C3 alkyl sulfanyl, or —(CH₂)ₚ—(CH₂)_{q}—C(O)OH, wherein and q are independently an integer from 1 to 6. More preferably, R¹⁰⁷ is C1-C6 alkyl or C1-C6 carboxyalkyl. The remainder of the variables take the values defined above in formula (I).

In alternative embodiment of the present invention, R alone or taken together with R⁴, or alternatively R⁵, and the intervening carbon atoms is a phenol isosteric group. As used herein, the term “phenol isosteric group” means a chemical moiety whose quantum properties result in such electrostatic charge distribution, polarizability, capacity to form hydrogen bonds, hydrophobicity, steric effect and other inductive or mesomeric effects, that the molecule as a whole is endowed with activity as either agonist or antagonist of receptors, enzyme active sites, and protein/enzyme allosteric modulation sites in the context of biological function.

In some embodiments, the phenol isosteric group is selected from the functional groups of formulas (X) — (XXIII):

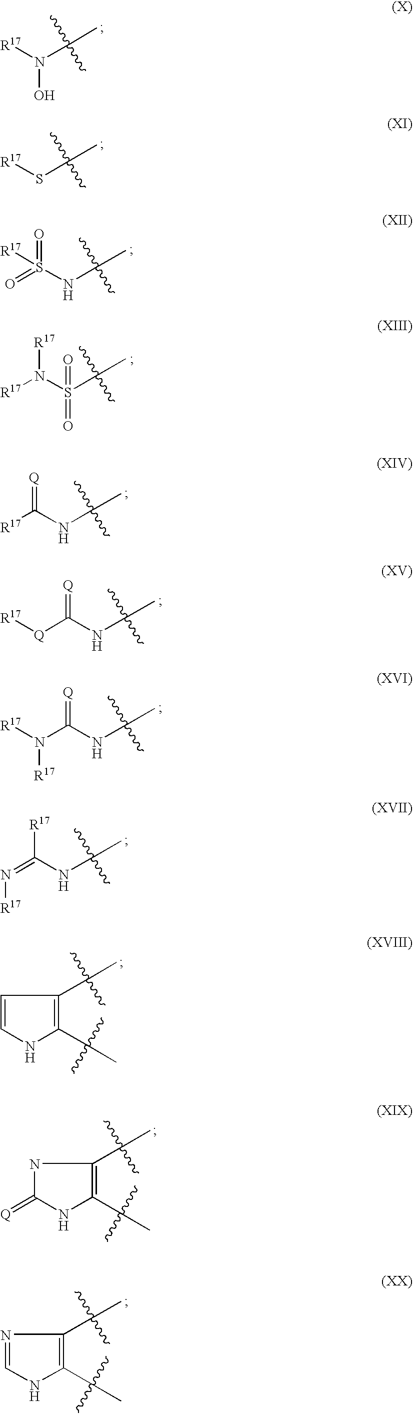

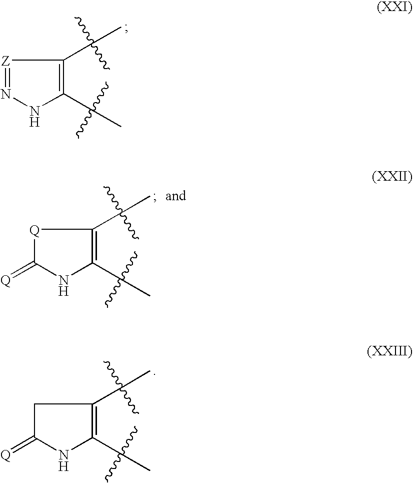

In formulas (X) — (XXIII), R¹⁷ is H, C1-C6 alkyl, C1-C6 alkoxyalkyl, optionally substituted aryl or aralkyl or heteroaryl, optionally substituted aryloxy, aralkyloxy or heteroaryloxy, Q is O or S, and Z is CH or N. The remainder of the variables take the values defined above in formula (I).

Preferably R¹⁷ is H, optionally substituted C1-C6 alkyl, optionally substituted C1-C6 alkoxyalkyl, or phenyl, benzyl, phenyloxy or benzyloxy, optionally substituted with one or more halogen atoms, —NO₂, —NH₂, —COOH, C1-C3 alkyl, C1-C3 carbalkoxy, C1-C3 alkoxy group, C1-C3 haloalkyl or C1-C3 haloalkoxy. More preferably, R¹⁷ is H, C1-C4 alkyl, or phenyl, optionally substituted with one or more halogen atoms or C1-C3 haloalkyls. Even more preferably, R¹⁷ is H, C1-C4 haloalkyl, or phenyl, optionally substituted with one or more C1-C3 haloalkyls. Examples of R¹⁷ include H, trifluoromethyl or phenyl substituted with one or more trifluoromethyls. The remainder of the variables take the values defined above in formula (I).

Examples of compounds of formula (I) include: 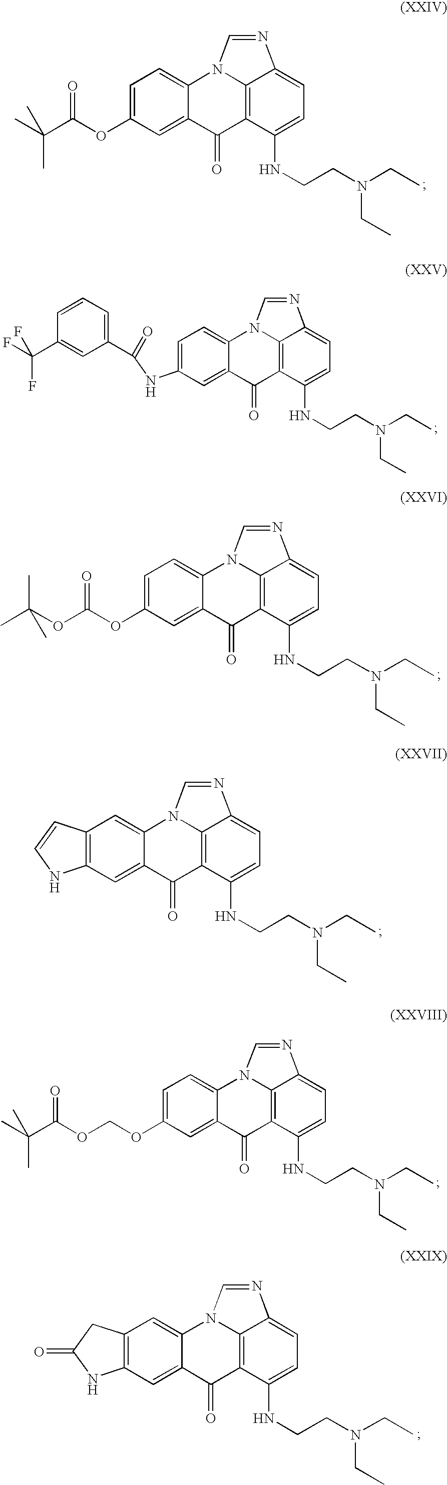 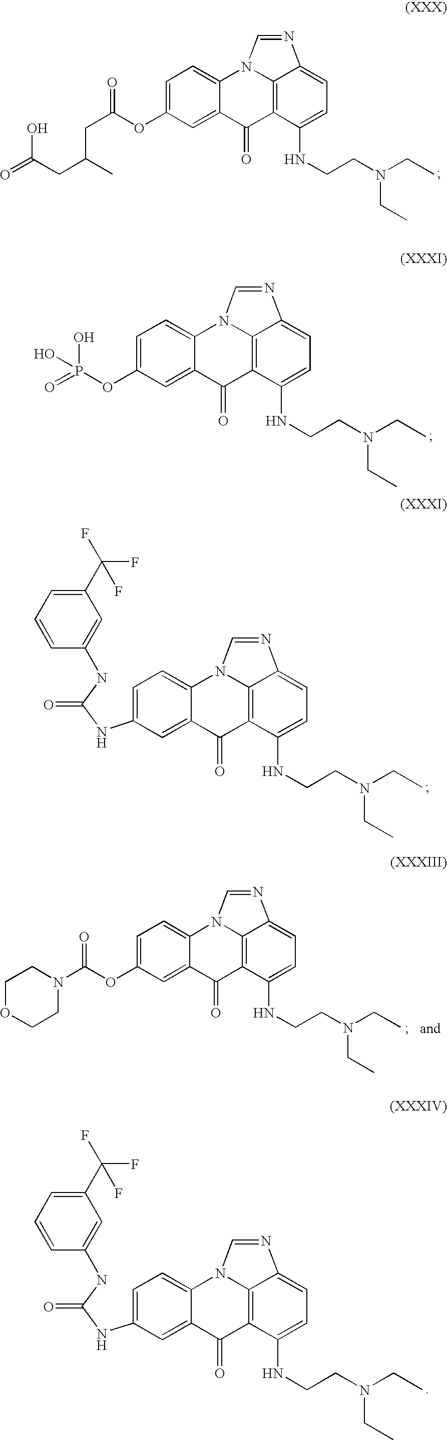
2. Indazoles

In one embodiment, the present invention is a compound of formula (IA) below or a pharmaceutically acceptable salt thereof. 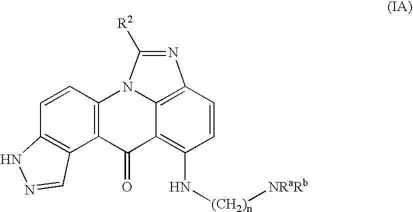
Values and preferred values for the variables in formula (IA) are defined below.

R² is —H or an optionally substituted C1-C4 alkyl. Preferably, R² is —H.

Integer n is from 2 to 5, preferably, 2 or 3. In another embodiment, n is 2; alternatively, n is 3. In another embodiment, n is 4; alternatively, n is 5.

R^{a} and R^{b}, each independently, are hydrogen or an optionally substituted alkyl, Alternatively, R^{a} and R^{b}, taken together with the nitrogen to which they are attached, form a non-aromatic heterocycle, each optionally substituted at one or more substitutable carbon atoms with methyl, hydroxyl, or methoxy, and optionally N′-substituted with C1-C4 alkyl or C1-C4 alkyl substituted with —NR^{c}R^{d}.

Preferably, R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}. Alternatively, R^{a} and R^{b} or individually are hydrogen or a C1-C3 alkyl optionally substituted with —OH, —SH, halogen, cyano, nitro, amino, —COOH, a C1-C3 alkyl.

More preferably, R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}.

More preferably, R^{a} and R^{b}, taken together with the nitrogen to which they are attached, form a 5-7 member non-aromatic heterocycle selected form a group consisting of:

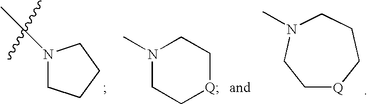

Q is S, O, CH₂, NH, or NR¹⁰², and R¹⁰² is methyl or ethyl. More preferably, R^{a} and R^{b}, taken together with the nitrogen to which they are attached, form N-morpholinyl or N-piperidinyl.

More preferably, R^{a} and R^{b} individually are hydrogen or a C1-C3 alkyl optionally substituted with —OH, —SH, halogen, cyano, nitro, amino, —COOH, a C1-C3 alkyl. More preferably, R^{a} and R^{b} individually are H, methyl or ethyl.

R^{c} and R^{d} are individually H, methyl or ethyl.

In a first preferred embodiment, R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}; or R^{a} and R^{b} or both are hydrogen or a C1-C3 alkyl optionally substituted with —OH, —SH, halogen, cyano, nitro, amino, —COOH, a C1-C3 alkyl. Preferably, R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}. Values and preferred values for the remainder of variables are as defined above in formula (IA).

In another embodiment, the compound of the invention is represented by formula (IIA):

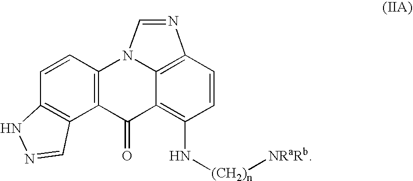

Values and preferred values for formula (IIA) are as described above for formula (IA).

In another embodiment, the compound of the invention is represented by formula (IIA), wherein: R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}. Values and preferred values for the remainder of variables are as defined above in formula (IA).

In another embodiment, the compound of the invention is represented by formula (IIA), wherein: n is 2 or 3; and R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}, and values and preferred values for the remainder of variables are as defined above in formula (IA). Preferably, R^{a} and R^{b}, taken together with the nitrogen to which they are attached, form a 5-7 member non-aromatic heterocycle selected form a group consisting of: 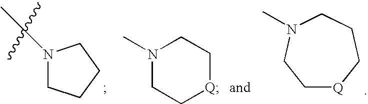
wherein Q is S, O, CH₂, NH, or NR¹⁰², and R¹⁰² is methyl or ethyl. More preferably, R^{a} and R^{b}, taken together with the nitrogen to which they are attached, form N-morpholinyl or N-piperidinyl.

In another embodiment, the compound of the invention is represented by formula (IIA), wherein: R^{a} and R^{b} individually are hydrogen or a C1-C3 alkyl optionally substituted with —OH, —SH, halogen, cyano, nitro, amino, —COOH, or C1-C3 alkyl; and the values and preferred values for the remainder of the variables are as described for formula (IA).

In another embodiment, the compound of the invention is represented by formula (II), wherein: n is 2 or 3 and R^{a} and R^{b} individually are hydrogen or a C1-C3 alkyl optionally substituted with —OH, —SH, halogen, cyano, nitro, amino, —COOH, or C1-C3 alkyl; and preferred values for the variables are as described for formula (I). More preferably, R^{a} and R^{b} both are H, methyl or ethyl.

Examples of compounds of the invention include: 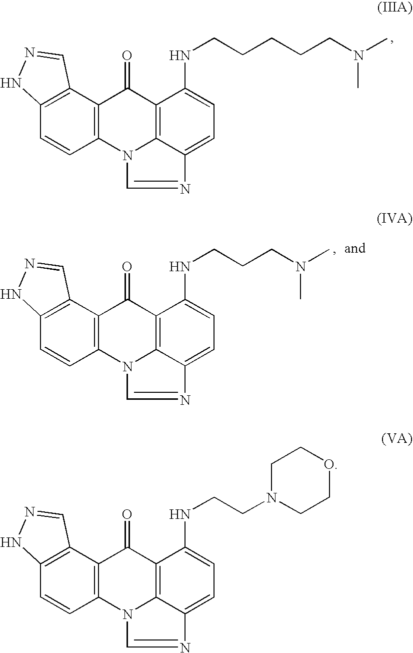
3. Esters and Carbonates

In one embodiment, the present invention is a compound of formula (IB) below or a pharmaceutically acceptable salt thereof. 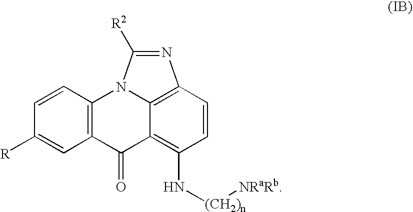
Values and preferred values for the substituents in formula (IB) are defined below.

R is 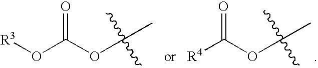
R² is —H or an optionally substituted C1-C4 alkyl. Preferably, R² is —H.

Integer n is from 2 to 5, preferably, 2 or 3. In another embodiment, n is 2; alternatively, n is 3. In another embodiment, n is 4; alternatively, n is 5.

R³ is an optionally substituted C1-C6 alkyl, C3-C8 cycloalkyl, or optionally substituted aryl, or a non-aromatic heterocycle, wherein the non-aromatic heterocycle or cycloalkyl are optionally and independently substituted at one or more substitutable carbon atoms with methyl, hydroxyl, or methoxy.

Preferably, R³ is C1-C6 alkyl optionally substituted with halogen, cyano, C1-C3 dialkylamino, C1-C3 alkyl, C1-C3 haloalkyl, C1-C3 alkoxy, C1-C3 haloalkoxy or C1-C3 alkoxycarbonyl or is C3-C6 cycloalkyl, optionally substituted with methyl, hydroxyl or methoxy. In another alternative, R³ is C3-C8 (preferably C3-C6) cycloalkyl optionally substituted at a substitutable ring carbon atom with methyl, hydroxyl or methoxy. In another alternative, R³ is a C3-C8 (preferably C3-C6) cycloalkyl. Alternatively, R³ is phenyl or benzyl optionally substituted with a halogen, C1-C3 dialkylamino, C1-C4 alkyl, C1-C3 alkoxycarbonyl, C1-C3 alkoxy, C1-C3 haloalkyl or C1-C3 haloalkoxy.

R⁴ is an optionally substituted C1-C6 alkyl, C3-C8 cycloalkyl, or optionally substituted aryl, or a non-aromatic heterocycle, wherein the non-aromatic heterocycle or cycloalkyl are optionally and independently substituted at one or more substitutable carbon atoms with methyl, hydroxyl, or methoxy.

Preferably, R⁴ is C1-C6 alkyl optionally substituted with halogen, cyano, C1-C3 dialkylamino, C1-C3 alkyl, C1-C3 haloalkyl, C1-C3 alkoxy, C1-C3 haloalkoxy or C1-C3 alkoxycarbonyl or is C3-C6 cycloalkyl, optionally substituted with methyl, hydroxyl or methoxy. In another alternative, R⁴ is C3-C8 (preferably C3-C6) cycloalkyl optionally substituted at a substitutable ring carbon atom with methyl, hydroxyl or methoxy. In another alternative, R⁴ is a C3-C8 (preferably C3-C6) cycloalkyl. Alternatively, R⁴ is phenyl or benzyl optionally substituted with a halogen, C1-C3 dialkylamino, C1-C4 alkyl, C1-C3 alkoxycarbonyl, C1-C3 alkoxy, C1-C3 haloalkyl or C1-C3 haloalkoxy.

R^{a} and R^{b}, each independently, are hydrogen or an optionally substituted alkyl, Alternatively, R^{a} and R^{b}, taken together with the nitrogen to which they are attached, form a non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}.

Preferably, R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}. Alternatively, R^{a} and R^{b} individually are hydrogen or a C1-C3 alkyl optionally substituted with —OH, —SH, halogen, cyano, nitro, amino, —COOH, a C1-C3 alkyl.

More preferably, R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}.

More preferably, R^{a} and R^{b}, taken together with the nitrogen to which they are attached, form a 5-7 member non-aromatic heterocycle selected form a group consisting of:

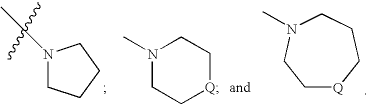

Q is S, O, CH₂, NH, or NR¹⁰², and R¹⁰² is methyl or ethyl. More preferably, R^{a} and R^{b}, taken together with the nitrogen to which they are attached, form N-morpholinyl or N-piperidinyl.

More preferably, R^{a} and R^{b} individually are hydrogen or a C1-C3 alkyl optionally substituted with —OH, —SH, halogen, cyano, nitro, amino, —COOH, a C1-C3 alkyl. More preferably, R^{a} and R^{b} individually are H, methyl or ethyl.

R^{c} and R^{d} are individually H, methyl or ethyl.

In a first preferred embodiment, the compound of formula (IB) is represented by a compound of formula (IIB): 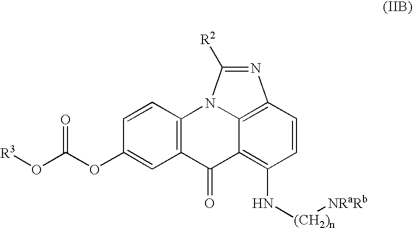
or a pharmaceutically acceptable salt thereof. Values and preferred values for the variables in formula (IIB) are as defined above in formula (IB).

Preferably, the compound is represented by formula (IIB), and R³ is C1-C6 alkyl optionally substituted with halogen, cyano, C1-C3 dialkylamino, C1-C3 alkyl, C1-C3 haloalkyl, C1-C3 alkoxy, C1-C3 haloalkoxy or C1-C3 alkoxycarbonyl or is C3-C6 cycloalkyl, optionally substituted with methyl, hydroxyl or methoxy. In another alternative, R³ is C3-C8 (preferably C3-C6) cycloalkyl optionally substituted at a substitutable ring carbon atom with methyl, hydroxyl or methoxy. In another alternative, R³ is a C3-C8 (preferably C3-C6) cycloalkyl. Values and preferred values for the remainder of variables are as defined above in formula (IB).

Alternatively, the compound is represented by formula (IIB), and R³ is phenyl or benzyl optionally substituted with a halogen, C1-C3 dialkylamino, C1-C4 alkyl, C1-C3 alkoxycarbonyl, C1-C3 alkoxy, C1-C3 haloalkyl or C1-C3 haloalkoxy. Values and preferred values for the remainder of variables are as defined above in formula (IB).

In a second preferred embodiment, the compound of formula (IB) is represented by a compound of formula (IIIB): 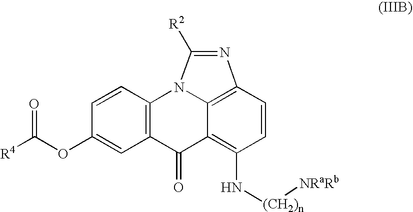
or a pharmaceutically acceptable salt thereof. Values and preferred values for the variables in formula (IIIB) are as defined above in formula (IB).

Preferably, the compound is represented by formula (IIIB), and R⁴ is C1-C6 alkyl optionally substituted with halogen, cyano, C1-C3 dialkylamino, C1-C3 alkyl, C1-C3 haloalkyl, C1-C3 alkoxy, C1-C3 haloalkoxy or C1-C3 alkoxycarbonyl or is C3-C6 cycloalkyl, optionally substituted with methyl, hydroxyl or methoxy. In another alternative, R⁴ is a C3-C8 (preferably C3-C6) cycloalkyl optionally substituted at a substitutable ring carbon atom with methyl, hydroxyl or methoxy. In another alternative, R⁴ is a C3-C8 (preferably C3-C6) cycloalkyl. Values and preferred values for the remainder of variables are as defined above in formula (I).

Alternatively, the compound is represented by formula (IIIB), and R⁴ is phenyl or benzyl optionally substituted with a halogen, C1-C3 dialkylamino, C1-C4 alkyl, C1-C3 alkoxycarbonyl, C1-C3 alkoxy, C1-C3 haloalkyl or C1-C3 haloalkoxy. Values and preferred values for the remainder of variables are as defined above in formula (IB).

Preferably, the compound of the present invention is represented by any of the formulas (IB), (IIB), and (IIIB), and R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}; or R^{a} and R^{b} or both are hydrogen or a C1-C3 alkyl optionally substituted with —OH, —SH, halogen, cyano, nitro, amino, —COOH, a C1-C3 alkyl. Values and preferred values for the remainder of variables are as defined above in formulas (IB), (IIB), and (IIIB).

More preferably, the compound of the present invention is represented by any of the formulas (IB), (IIB), and (IIIB), and R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}. Values and preferred values for the remainder of variables are as defined above in formula (IB), (IIB), and (IIIB).

In one preferred embodiment, the compounds of formula (IB) are represented by formulas (IVB-a) or (IVB-b) or a pharmaceutically acceptable salt thereof: 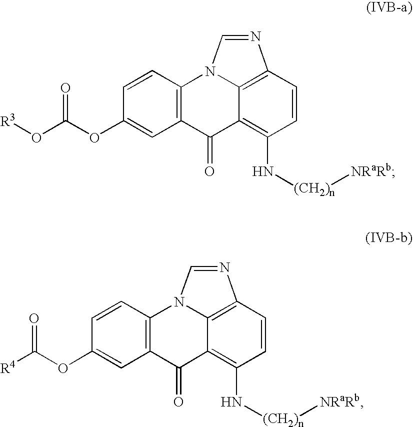
and R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}. Values and preferred values for the remainder of variables are as defined above in formula (IB), (IIB), and (IIIB).

In another preferred embodiment, the compound of formula (IB) is represented by formulas (IVB-a) or (IVB-b), R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}, and n is 2 or 3. Values and preferred values for the remainder of variables are as defined above in formula (IB), (IIB), and (IIIB).

In another preferred embodiment, the compound of formula (IB) is represented by formulas (IVB-a) or (IVB-b), n is 2 or 3, and R^{a} and R^{b}, taken together with the nitrogen to which they are attached, form a 5-7 member non-aromatic heterocycle selected form a group consisting of 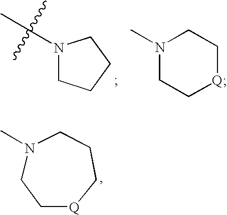
wherein Q is S, O, CH₂, NH, or NR¹⁰², and R¹⁰² is methyl or ethyl. Values and preferred values for the remainder of variables are as defined above in formula (IB), (IIB), and (IIIB).

In another preferred embodiment, the compound of formula (IB) is represented by formulas (IVB-a) or (IVB-b), n is 2 or 3, and R^{a} and R^{b}, taken together with the nitrogen to which they are attached, form N-morpholinyl or N-piperidinyl. Values and preferred values for the remainder of variables are as defined above in formula (IB), (IIB), and (IIIB).

More preferably, the compound of the present invention is represented by any of the formulas (IB), (IIB), and (IIIB), and R^{a} and R^{b} both are hydrogen or a C1-C3 alkyl optionally substituted with —OH, —SH, halogen, cyano, nitro, amino, —COOH, a C1-C3 alkyl. Values and preferred values for the remainder of variables are as defined above in formula (IB), (IIB), and (IIIB).

In one preferred embodiment, the compound of formula (I) is represented by formulas (IVB-a) or (IVB-b), above, and R^{a} and R^{b} both are hydrogen or a C1-C3 alkyl optionally substituted with —OH, —SH, halogen, cyano, nitro, amino, —COOH, a C1-C3 alkyl. Values and preferred values for the remainder of variables are as defined above in formula (IB), (IIB), and (IIIB).

In another preferred embodiment, the compound of formula (IB) is represented by formulas (IVB-a) or (IVB-b), above, R^{a} and R^{b} both are hydrogen or a C1-C3 alkyl optionally substituted with —OH, —SH, halogen, cyano, nitro, amino, —COOH, a C1-C3 alkyl, and n is 2 or 3. Values and preferred values for the remainder of variables are as defined above in formula (IB), (IIB), and (IIIB).

In another preferred embodiment, the compound of formula (IB) is represented by formulas (IVB-a) or (IVB-b), above, n is 2 or 3, and R^{a} and R^{b} both are H, methyl or ethyl. Values and preferred values for the remainder of variables are as defined above in formula (IB), (IIB), and (IIIB).

Examples of compounds of formula (IB) include compounds represented by the following formulas: 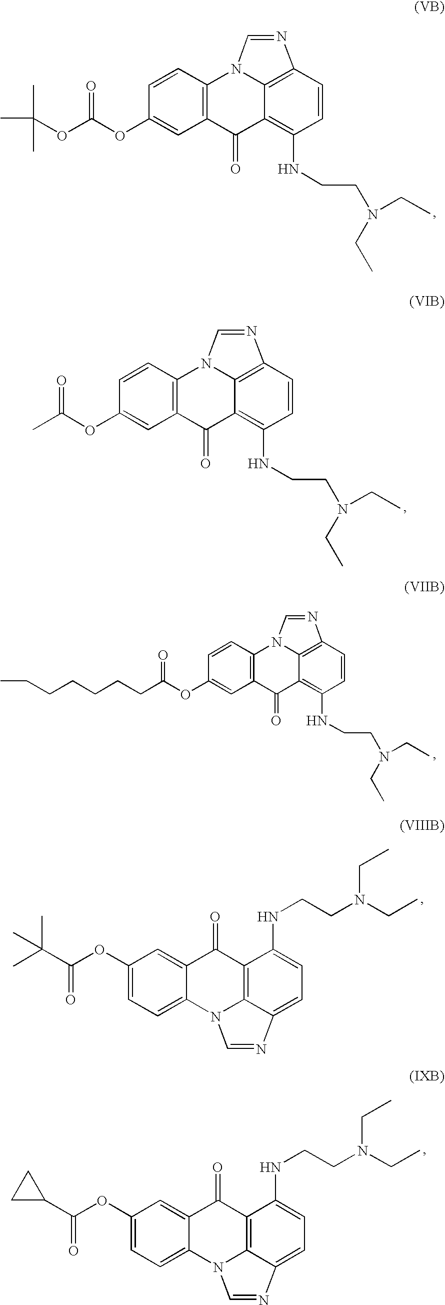 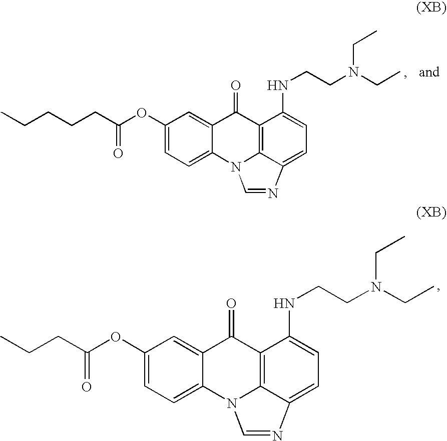
or a pharmaceutically acceptable salt thereof.

Additional examples of the compounds of formula (IB) include compounds represented by formulas (XXIV), (XXVI), and (XXX), mentioned above:

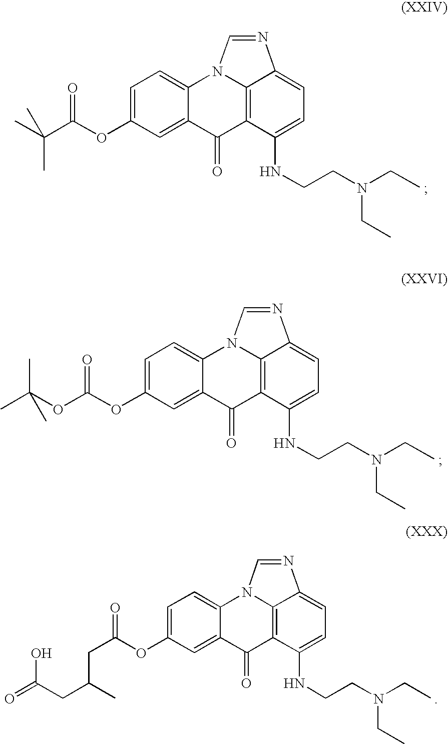

### DEFINITION OF TERMS

The term “alkyl”, as used herein, unless otherwise indicated, includes straight or branched saturated monovalent hydrocarbon radicals, typically C1-C10, preferably C1-C6. Examples of alkyl groups include, but are not limited to, methyl, ethyl, propyl, isopropyl, and t-butyl. Suitable substituents for a substituted alkyl include —OH, —SH, halogen, cyano, nitro, amino, —COOH, a C1-C3 alkyl, C1-C3 haloalkyl, C1-C3 alkoxy, C1-C3 haloalkoxy or C1-C3 alkyl sulfanyl, or —(CH₂)ₚ—(CH₂)_{q}—C(O)OH, where p and q are independently an integer from 1 to 6.

The term “cycloalkyl”, as used herein, is a non-aromatic saturated carbocyclic moieties. Examples of cycloalkyl include, but are not limited to, cyclopropyl, cyclobutyl, cyclopentyl, cyclohexyl, and cycloheptyl. Suitable substituents for a cycloalkyl are defined above for an alkyl.

The term “haloalkyl”, as used herein, includes an alkyl substituted with one or more F, Cl, Br, or I, wherein alkyl is defined above.

The terms “alkoxy”, as used herein, means an “alkyl-O—” group, wherein alkyl, is defined above.

The term “alkanoyl”, as used herein, means an “alkyl-C(O)-” group, wherein alkyl is defined above.

The term “haloalkoxy”, as used herein, means “haloalkyl-O—”, wherein haloalkyl is defined above.

As used herein, an amino group may be a primary (—NH₂), secondary (—NHRₓ), or tertiary (—NRₓR_{y}), wherein Rₓ and R_{y} may be any of the optionally substituted alkyls alkyls described above.

The term “aryl”, as used herein, refers to a carbocyclic aromatic group. Examples of aryl groups include, but are not limited to phenyl and naphthyl.

The term “aryloxy”, as used herein, means an “aryl-O—” group, wherein aryl is defined above.

The term “aroyl”, as used herein, means an “aryl-C(O)—” group, wherein aryl is defined above.

The term “heteroaryl”, as used herein, refers to aromatic groups containing one or more heteroatoms (O, S, or N). A heteroaryl group can be monocyclic or polycyclic, e.g. a monocyclic heteroaryl ring fused to one or more carbocyclic aromatic groups or other monocyclic heteroaryl groups. The heteroaryl groups of this invention can also include ring systems substituted with one or more oxo moieties. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

The term “heteroaryloxy”, as used herein, means a “heteroaryl-O—” group, wherein heteroaryl is defined above.

The term “non-aromatic heterocycle” refers to non-aromatic carbocyclic ring systems typically having four to eight members, preferably five to six, in which one or more ring carbons, preferably one to four, are each replaced by a heteroatom such as N, O, or S, Non aromatic heteroccyles can be optionally unsaturated. Examples of non-aromatic heterocyclic rings include 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrorolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, and 1-pthalimidinyl.

The heteroaryl or non-aromatic heterocyclic groups may be C-attached or N-attached (where such is possible). For instance, a group derived from pyrrole may be pyrrol-1-yl (N-attached) or pyrrol-3-yl (C-attached).

Suitable substituents for an aryl, a heteroaryl, or a non-aromatic heterocyclic group are those that do not substantially interfere with the pharmaceutical activity of the disclosed compound. One or more substituents can be present, which can be identical or different. Examples of suitable substituents for a substitutable carbon atom in aryl, heteroaryl or a non-aromatic heterocyclic group include —OH, halogen (—F, —Cl, —Br, and —I), —R′, haloalkyl, —OR′, —CH₂R′, —CH₂OR′, —CH₂CH₂OR′, —CH₂OC(O)R′, —O—COR′, —COR′, —SR′, —SCH₂R′, —CH₂SR′, —SOR′, —SO₂R′, —CN, —NO₂, —COOH, —SO₃H, —NH₂, —NHR′, —N(R′)₂, —COOR′, —CH₂COOR′, —CH₂CH₂COOR′, —CHO, —CONH₂, —CONHR′, —CON(R′)₂, —NHCOR′, —NR′COR′, —NHCONH₂, —NHCONR′H, —NHCON(R′)₂, —NR′CONH₂, —NR′CONR′H, —NR′CON(R′)₂, —C(═NH)—NH₂, —C(═NH)—NHR′, —C(═NH)—N(R′)₂, —C(═NR′)—NH₂, —C(═NR′)—NHR′, —C(═NR′)—N(R′)₂, —NH—C(═NH)—NH₂, —NH—C(═NH)—NHR′, —NH—C(═NH)—N(R′)₂, —NH—C(═NR′)—NH₂, —NH—C(═NR′)—NHR′, —NH—C(═NR′)—N(R′)₂, —NR′H—C(═NH)—NH₂, —NR′—C(═NH)—NHR′, —NR′—C(═NH)—N(R′)₂, —NR′—C(═NR′)—NH₂, —NR′—C(═NR′)—NHR′, —NR′—C(═NR′)—N(R′)₂, —SO₂NH₂, —SO₂NHR′, —SO₂NR₁₂, —SH, —SOₖR′(k is 0, 1 or 2) and —NH—C(═NH)—NH₂. Each R′ is independently an alkyl group. Oxo (C═O) and thio (C═S) are also suitable substituents for a non-aromatic heterocycle.

Suitable substituents on the nitrogen of a non-aromatic heterocyclic group or a heteroaryl group include —R″, —N(R″)₂, —C(O)R″, —CO₂ R″, —C(O)C(O)R″, —C(O)CH₂ C(O)R″, —SO₂R″, —SO₂ N(R″)₂, —C(═S)N(R″)₂, —C(═NH)—N(R″)₂, and —NR″ SO₂R¹⁵. R″ is hydrogen, an alkyl or alkoxy group.

Synthesis of the Compounds of the Present Invention

Imidazoacridines of Formula (I)

Compounds of formula (I) can be synthesized according to a variety of synthetic schemes disclosed in U.S. Pat. Nos. 5,231,100 and 6,229,015, incorporated herein by reference in their entirety. One example of such a scheme is shown below:

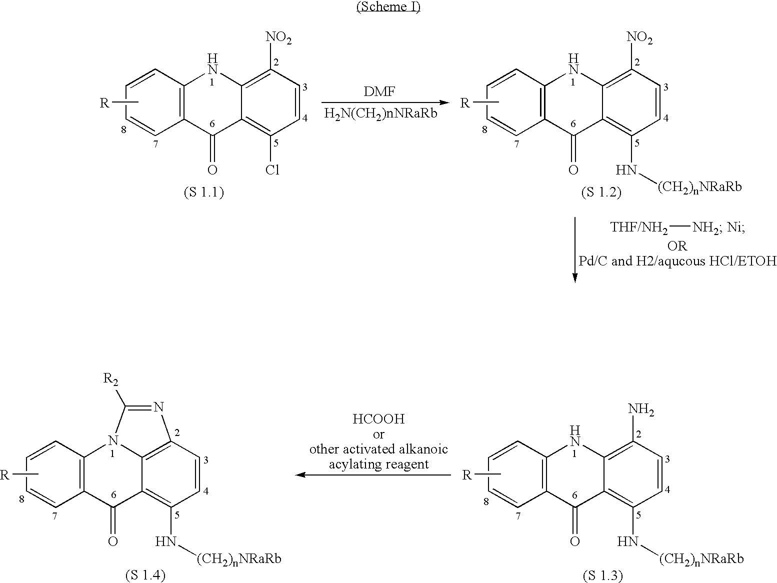

As used herein, the “activated alkanoic acylating agent” is defined within the references cited. For example: alkyl nitrites are reacted with HCl in methanol or ethanol to give the corresponding acetimidate ester hydrochlorides, R—CN going to R—C(OMe)═NH+Cl—, where R is an alkyl. This acetimidate reacts with the amine in compound (S I.3) and cyclized to the methyl or ethyl or other alkylimidazole, again per the cited articles.

Indazoles of Formula (IA)

Synthesis of the indazoles of formula (IA) is illustrated in Example 8 below (see EXEMPLIFICATION).

Esters and Carbonate of Formula (IB)

Synthesis of the esters and carbonates of the formula (IB) is illustrated in Example 10 below (see EXEMPLIFICATION).

Methods of Use of the Compounds of the Invention

It has now been discovered, that Symadex™ inhibits proliferation of B-cells following stimulation with LPS and T-cells following stimulation with Con A as well as that Symadex™ inhibit release of cytokines such as IL-4 and IL-10 (Example 1). It has further been discovered in microarray experiments, that Symadex™ treatment results in altered expression of several genes involved in key regulatory pathways affecting the inflammatory and proliferative states, particularly the ability of invasive cells to assemble and aggregate, downregulation of cell proliferation and cell-cell signaling (Example 3). These molecular pharmacology studies show that Symadex™ exerts a downregulatory effect on genes implicated in mechanisms of cell aggregation and proliferation and on processes associated with invasive cellular growth, which are the hallmark of the inflammatory etiology associated with the autoimmune diseases. Taken together, these results indicate that Symadex™ can be used for treating the disorders that have inflammatory component, including autoimmune diseases.

It was further discovered that Symadex™ demonstrates activity in the female Hartley guinea pig Experimental Autoimmune Encephalomyelitis (EAE) model, a classic animal model for chronic-progressive MS (Example 2). Taken together with the results of Example 3, this result indicates that Symadex™ can be used for treating the disorders that have demyelinating as well as inflammatory components.

Accordingly, in one embodiment, the present invention is a method of treating a patient suffering from an inflammatory condition. The condition can be systemic lupus, inflammatory bowl disease, psoriasis, Crohn's disease, rheumatoid arthritis, sarcoid, Alzheimer's disease, a chronic inflammatory demyelinating neuropathy, insulin dependent diabetes mellitus, atherosclerosis, asthma, spinal cord injury or stroke.

Examples of chronic inflammatory demyelinating neuropathies include: chronic Immune Demyelinating Polyneuropathy (CIDP); multifocal CIDP; multifocal motor neuropathy (MMN); anti-MAG Syndrome (Neuropathy with IgM binding to Myelin-Associated Glycoprotein); GALOP Syndrome (Gait disorder Autoantibody Late-age Onset Polyneuropathy); anti-sulfatide antibody syndrome; anti-GM2 gangliosides antibody syndrome; POEMS syndrome (Polyneuropathy Organomegaly Endocrinopathy or Edema M-protein Skin changes); perineuritis; and IgM anti-GD1b ganglioside antibody syndrome.

The method comprises administering to a patient a therapeutically effective amount of a compounds of formulas (I), (IA), and (IB) or a pharmaceutically acceptable salt thereof.

In another embodiment, the present invention is a method of treatment of a patient suffering from a demyelinating condition. As used herein, a “demyelinating condition” is a condition that destroys, breaks the integrity of or damages a myelin sheath. As used herein, the term “myelin sheath” refers to an insulating layer surrounding vertebrate peripheral neurons, that increases the speed of conduction and formed by Schwann cells in the peripheral or by oligodendrocytes in the central nervous system. Such condition can be multiple sclerosis, a congenital metabolic disorder, a neuropathy with abnormal myelination, drug-induced demyelination, radiation induced demyelination, a hereditary demyelination condition, a prion-induced demyelination, encephalitis-induced demyelination, a spinal cord injury, Alzheimer's disease as well as chronic inflammatory demyelinating neuropathies, examples of which are given above.

In one embodiment, the condition is multiple sclerosis. The method comprises administering to a patient a therapeutically effective amount of a compound of formulas (I), (IA), and (IB) or a pharmaceutically acceptable salt thereof. The term “patient” means a warm blooded animal, such as for example rat, mice, dogs, cats, guinea pigs, and primates such as humans. The terms “treat” or “treating” include any treatment, including, but not limited to, alleviating symptoms, eliminating the causation of the symptoms either on a temporary or permanent basis, or preventing or slowing the appearance of symptoms and progression of the named disorder or condition. The term “therapeutically effective amount” means an amount of the compound, which is effective in treating the named disorder or condition. In certain embodiments, therapeutically effective amount means an amount sufficient to effect remyelination of nerve cells in a patient.

In another embodiment, the present invention is a method of promoting remyelination of nerve cells in a patient, comprising administering to the patient in need thereof a therapeutically effective amount of a compound of formulas (I), (IA), and (IB). The patient can be suffering from any of the demyelinating conditions listed above.

In another embodiment, the present invention is a method of preventing demyelination and promoting remyelination in a patient in need thereof, comprising administering a combination of a therapeutically effective amount of a compound of formulas (I), (IA), and (IB) or pharmaceutically acceptable salt thereof, and an anti-inflammatory agent as described below.

In another embodiment, the present invention is a method of reversing paralysis in a subject in need thereof with a demyelinating disease, comprising administering to the subject a compound in an amount sufficient to inhibit lymphocyte infiltration of immune cells in the spinal cord to promote remyelination of nerve cells in the spinal cord and thereby treating paralysis in said subject, wherein the compound is of formula formulas (I), (IA), and (IB) or a pharmaceutically acceptable salt thereof.

The dosage range at which the disclosed compounds of formulas (I), (IA), and (IB) exhibit their ability to act therapeutically can vary depending upon the severity of the condition, the patient, the formulation, other underlying disease states that the patient is suffering from, and other medications that may be concurrently administered to the patient. Generally, the inventive compounds of the invention will exhibit their therapeutic activities at dosages of between about 0.001 mg/kg of patient body weight/day to about 100 mg/kg of patient body weight/day. For example, the dosage can be 0.1-100 mg/kg, 1-100 mg/kg, 10-100 mg/kg, 1-50 mg, kg, 10-50 mg/kg or 10-30 mg/kg per day, per every other day or per week.

In other embodiments, compounds can be administered by any of the routes described below, preferably intravenously, in an amount from 1 mg per kilogram body weight to 20 mg per kg body weight. Compounds can be administered daily, once every 72 hours or weekly.

In one embodiment in which compounds are used to treat rheumatoid arthritis, compounds can be administered orally in an amount of 1-50 mg/kg, 10-40 mg/kg, 20-30 mg/kg or 30 mg per kilogram of body weight per day, per every other day or per week.

In one embodiment, the compounds of the invention are administered chronically to the patient in need thereof. For example, the chronic administration of the compound is daily, weekly, biweekly, or monthly over a period of at least one year, at least two years, at least three or more years.

In one embodiment, the compounds of formulas (I), (IA), and (IB) are administered intravenously in the amount of 1.5-30 mg/kg once at intervals of 1-3 months. In another embodiment, the compounds are administered orally in the amount of 5-100 mg/kg on same schedule as above. Alternatively, the compounds of formulas (I), (IA), and (IB) are administered several times over a period of up to 3 months and up to a cumulative dose of between 1.5 and 30 mg/kg. In another embodiment, the cumulative dose is from 5 to 100 mg/kg.

In another embodiment, the compounds of formulas (I), (IA), and (IB) are administered intravenously in the amount of 2.5-10 mg/kg weekly for 8-24 weeks, repeating as needed after 6-18 weeks off drug. Alternatively, the compounds of formulas (I), (IA), and (IB) are administered several times over a period of from 14 weeks to 42 weeks to achieve a cumulative dose from 20 mg/kg to 240 mg/kg. Administration can be repeated over one or more periods of 14-42 weeks.

In another embodiment, the compounds of formulas (I), (IA), and (IB) are administered intravenously in the amount of 2.5-10 mg/kg twice, 72 hrs apart for 1 to 2 weeks, repeating monthly. Alternatively, the compounds of formulas (I), (IA), and (IB) are administered several times over a period of up to two weeks, up to a cumulative dose of from 11 mg/kg to 47 mg/kg. Administration can be repeated monthly.

In another embodiment, the compounds of formulas (I), (IA), and (IB) are administered orally in the amount of 1-3 mg/kg daily for 10-15 days, repeating every 30-45 days. Alternatively, the compounds of formulas (I), (IA), and (IB) are administered several times over a period of up to 40-60 days, up to a cumulative dose of from 10 mg/kg to 45 mg/kg. Administration can be repeated over one or more periods of up to 40-60 days.

In another embodiment, the compounds of the invention are administered orally in the amount of 2-6 mg/kg daily for 3 days per week, repeating every 15-30 days. Alternatively, the compounds of formulas (I), (IA), and (IB) are administered several times over a period of up to 30 days up to a cumulative dose of 6-18 mg/kg. Administration can be repeated over one or more periods of up to 30 days.

Preferably, the administration of the compounds or the combinations of the compounds described herein results in an effective blood level of the compound in the patient of more than or equal to 10 ng/ml. For example, compounds can be administered intravenously in an amount of 20 μg to about 500 μg per kilogram body weight of the patient.

Preferred human doses for treating chronic (remitting-relapsing) multiple sclerosis (MS) are 0.1 mg/kg to 10 mg/kg, 1-10 mg/kg, 1-5 mg/kg, 2-7 mg/kg, 2-5 mg/kg. Schedule could be once a month, twice a month, three times a month or once or twice a week for 3 months, 6 month, 12 months or more.

Preferred human doses for treating acute MS, is 0.1 mg/kg to 10 mg/kg, 0.1-5 mg/kg, 0.1-2 mg/kg, 0.5-2 mg/kg or 0.5-1 mg/kg three times a day, twice a day, or daily, on a weekly, biweekly or monthly basis.

Preferred human doses for treating rheumatoid arthritis 0.1 mg/kg to 10 mg/kg, 1-10 mg/kg, 1-5 mg/kg, 2-7 mg/kg, 2-5 mg/kg three times a day, twice a day, or daily, on a weekly, biweekly or monthly basis.

The compounds used in the present invention can be administered alone or in combination with one or more other pharmaceutically active agents that are effective against the inflammatory condition and/or the demyelating disorder being treated.

As used herein, the term “combination” with reference to pharmaceutically active agents and the term “co-administering” and “co-administration” refer to administering more than one pharmaceutically active agent to a patient during one treatment cycle and not necessarily simultaneous or in a mixture.

In one embodiment, the compounds of the present invention are administered in combination with an anti-inflammatory agent. The anti-inflammatory agent can be adrenocorticotropic hormone, a corticosteroid, an interferon, glatiramer acetate, or a non-steroidal anti-inflammatory drug (NSAID).

Examples of suitable anti-inflammatory agents include corticosteroid such as prednisone, methylprednisolone, dexamethasone cortisol, cortisone, fludrocortisone, prednisolone, 6α-methylprednisolone, triamcinolone, or betamethasone.

Other examples of suitable anti-inflammatory agents include NSAIDs such as aminoarylcarboxylic acid derivatives (e.g., Enfenamic Acid, Etofenamate, Flufenamic Acid, Isonixin, Meclofenamic Acid, Niflumic Acid, Talniflumate, Terofenamate and Tolfenamic Acid), arylacetic acid derivatives (e.g., Acematicin, Alclofenac, Amfenac, Bufexamac, Caprofen, Cinmetacin, Clopirac, Diclofenac, Diclofenac Sodium, Etodolac, Felbinac, Fenclofenac, Fenclorac, Fenclozic Acid, Fenoprofen, Fentiazac, Flubiprofen, Glucametacin, Ibufenac, Ibuprofen, Indomethacin, Isofezolac, Isoxepac, Ketoprofen, Lonazolac, Metiazinic Acid, Naproxen, Oxametacine, Proglumrtacin, Sulindac, Tenidap, Tiramide, Tolectin, Tolmetin, Zomax and Zomepirac), arylbutyric acid ferivatives (e.g., Bumadizon, Butibufen, Fenbufen and Xenbucin) arylcarboxylic acids (e.g., Clidanac, Ketorolac and Tinoridine), arylproprionic acid derivatives (e.g., Alminoprofen, Benoxaprofen, Bucloxic Acid, Carprofen, Fenoprofen, Flunoxaprofen, Flurbiprofen, Ibuprofen, Ibuproxam, Indoprofen, Ketoprofen, Loxoprofen, Miroprofen, Naproxen, Oxaprozin, Piketoprofen, Piroprofen, Pranoprofen, Protinizinic Acid, Suprofen and Tiaprofenic Acid), pyrazoles (e.g., Difenamizole and Epirizole), pyrazolones (e.g., Apazone, Benzpiperylon, Feprazone, Mofebutazone, Morazone, Oxyphenbutazone, Phenylbutazone, Pipebuzone, Propyphenazone, Ramifenazone, Suxibuzone and Thiazolinobutazone), salicyclic acid derivatives (e.g., Acetaminosalol, 5-Aminosalicylic Acid, Aspirin, Benorylate, Biphenyl Aspirin, Bromosaligenin, Calcium Acetylsalicylate, Diflunisal, Etersalate, Fendosal, Flufenisal, Gentisic Acid, Glycol Salicylate, Imidazole Salicylate, Lysine Acetylsalicylate, Mesalamine, Morpholine Salicylate, 1-Naphthyl Sallicylate, Olsalazine, Parsalmide, Phenyl Acetylsalicylate, Phenyl Salicylate, 2-Phosphonoxybenzoic Acid, Salacetamide, Salicylamide O-Acetic Acid, Salicylic Acid, Salicyloyl Salicylic Acid, Salicylsulfuric Acid, Salsalate and Sulfasalazine), thiazinecarboxamides (e.g., Droxicam, Isoxicam, Piroxicam and Tenoxicam), ε-Acetamidocaproic Acid, S-Adenosylmethionine, 3-Amino-4-hydroxybutyric Acid, Amixetrine, Bendazac, Benzydamine, Bucolome, Difenpiramide, Ditazol, Emorfazone, Guaiazulene, Ketorolac, Meclofenamic Acid, Mefenamic Acid, Nabumetone, Nimesulide, Orgotein, Oxaceprol, Paranyline, Perisoxal, Pifoxime, Piroxicam, Proquazone, Tenidap and a COX-2 inhibitor (e.g., Rofecoxib, Valdecoxib and Celecoxib).

Further examples of anti-inflammatory agents include aspirin, a sodium salicylate, choline magnesium trisalicylate, salsalate, diflunisal, sulfasalazine, olsalazine, a para-aminophenol derivatives, an indole, an indene acetic acid, a heteroaryl acetic acid, an anthranilic acid, an enolic acid, an alkanones, a diaryl-substituted furanone, a diaryl-substituted pyrazoles, an indole acetic acids, or a sulfonanilide.

In some embodiments, the compounds of the present invention can be administered in combination with immunotherapeutic agents such as interferons and anti-integrin blocking antibodies like natalizumab.

Examples of agents suitable for treating demyelinating disorders include Pirfenidone, Epalrestat, Nefazodone hydrochloride, Memantine hydrochloride, Mitoxantrone hydrochloride, Mitozantrone hydrochloride, Thalidomide, Roquinimex, Venlafaxine hydrochloride, Intaxel, Paclitaxel, recombinant human nerve growth factor; nerve growth factor, ibudilast, Cladribine, Beraprost sodium, Levacecamine hydrochloride; Acetyl-L-camitine hydrochloride; Levocarnitine acetyl hydrochloride, Droxidopa, interferon alfa, natural interferon alpha, human lymphoblastoid interferon, interferon beta-1b, interferon beta-Ser, Alemtuzumab, Mycophenolate mofetil, Zoledronic acid monohydrate, Adapalene, Eliprodil, Donepezil hydrochloride, Dexanabinol, Dexanabinone, Xaliproden hydrochloride, interferon alfa-n3, lipoic acid, thioctic acid, Teriflunomide, Atorvastatin, Pymadin, 4-Aminopyridine, Fampridine, Fidarestat, Priliximab, Pixantrone maleate, Dacliximab, Daclizumab, Glatiramer acetate, Rituximab, Fingolimod hydrochloride, interferon beta-1a, Natalizumab, Abatacept, Temsirolimus, Lenercept, Ruboxistaurin mesilate hydrate, Dextromethorphan/quinidine sulfate, Capsaicin, Dimethylfumarate or Dronabinol/cannabidiol.

In some embodiments, the compounds of the present invention can be administered in combination with one or more other pharmaceutically active agents that are effective against multiple sclerosis. Examples of such agents include the interferons (interferon beta 1-a, beta 1-b, and alpha), glatiramer acetate or corticosteroids such as methylprednisolone and prednisone as well as chemotherapeutic agents such as mitoxantrone, methotrexate, azathioprine, cladribine cyclophosphamide, cyclosporine and tysabri.

Further examples of pharmaceutically active agents that are effective against multiple sclerosis and are suitable to be administered in combination with compounds of the present invention include compounds of the following structural formulae:

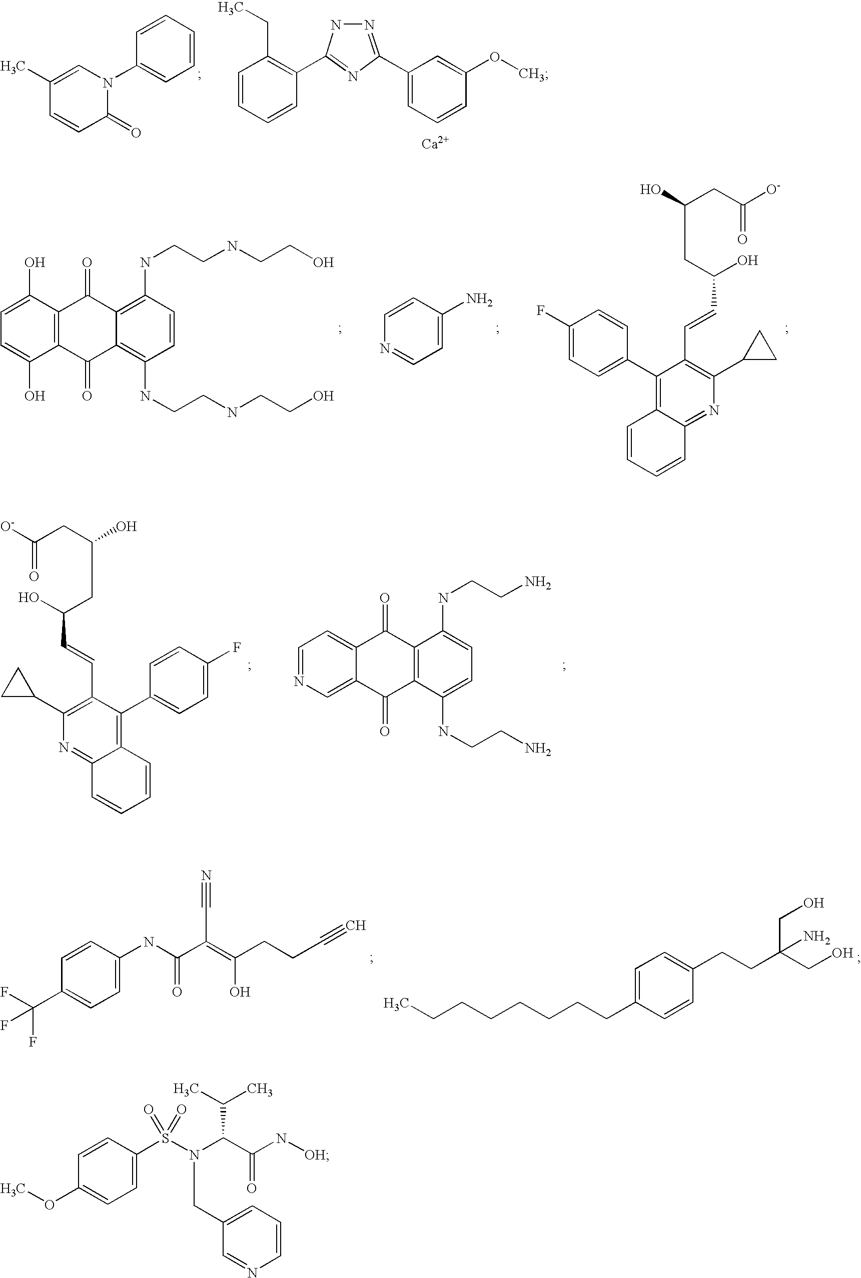

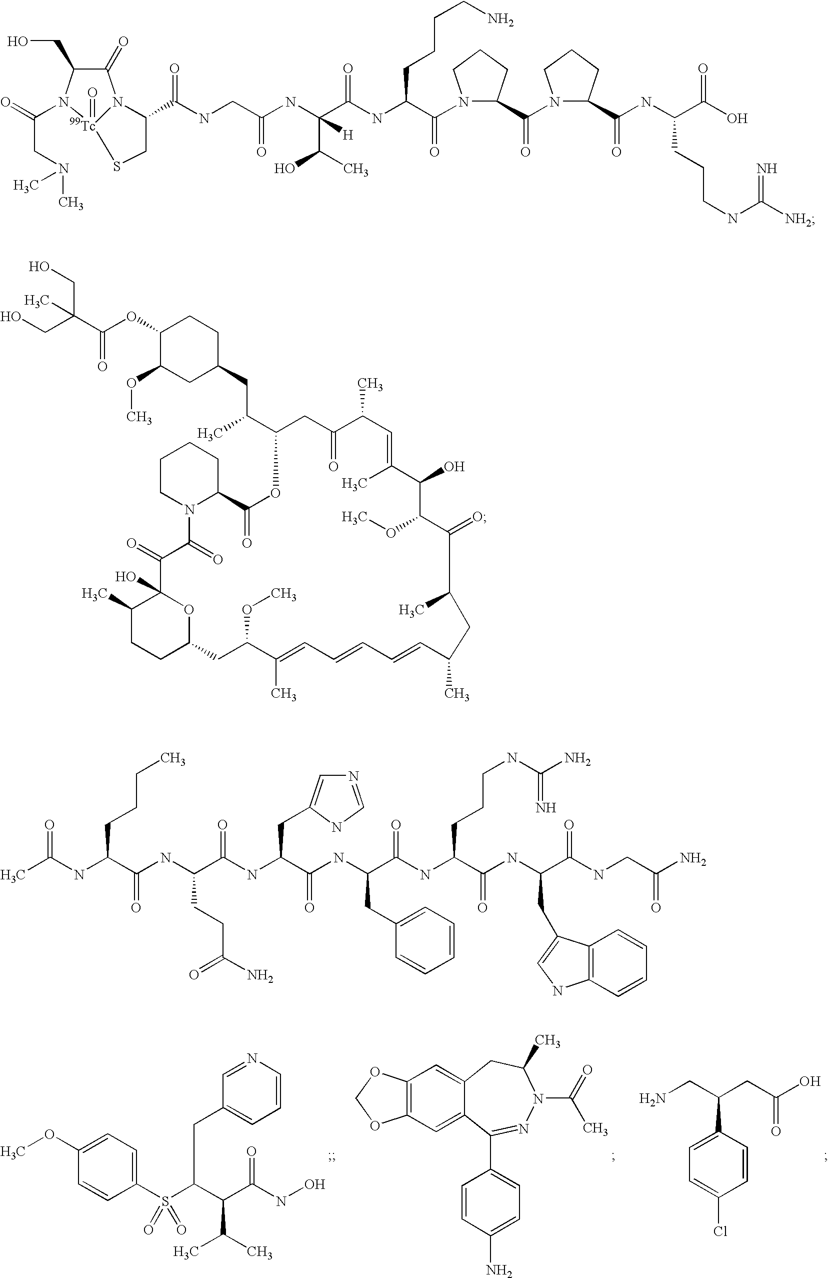

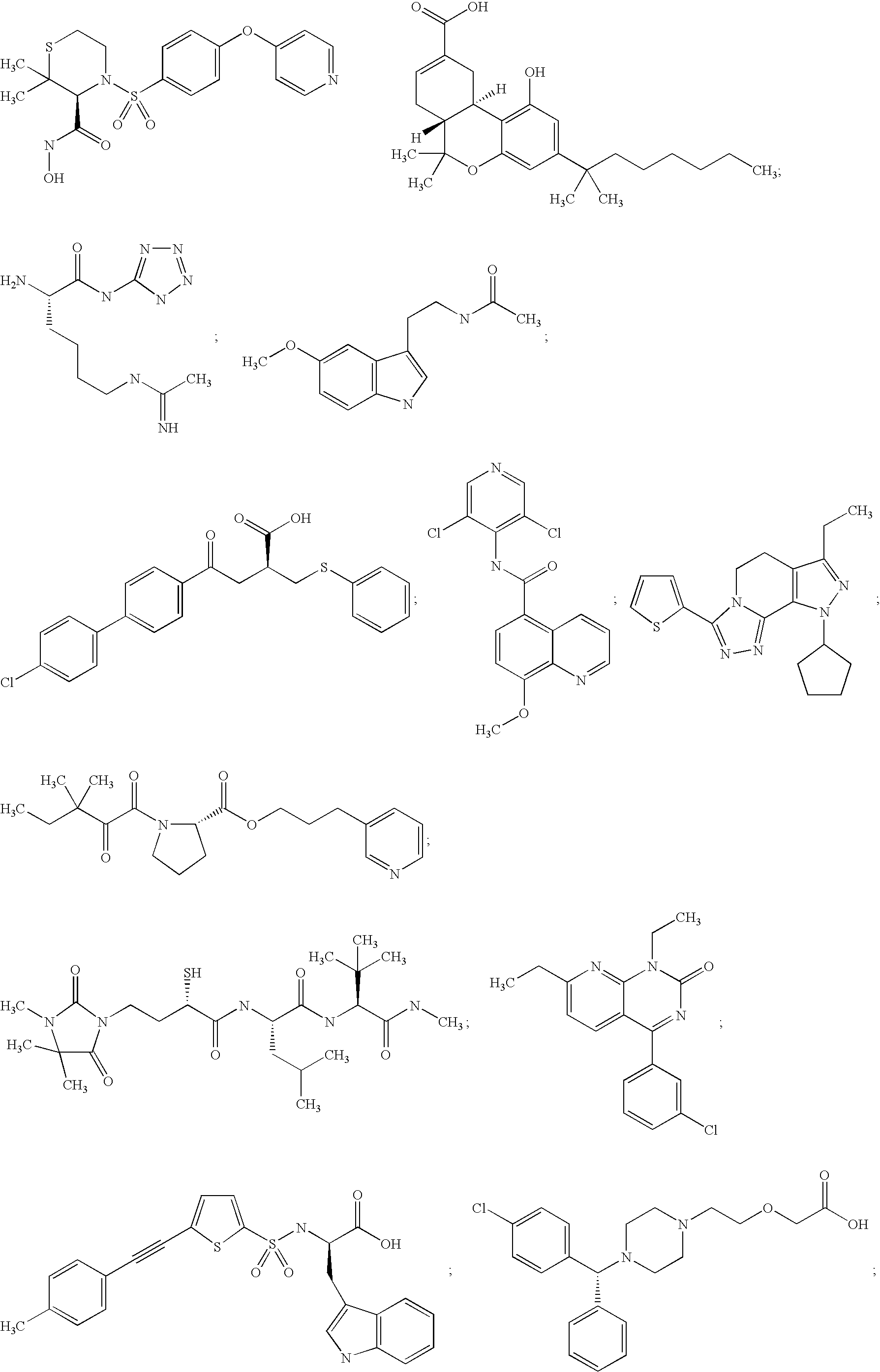

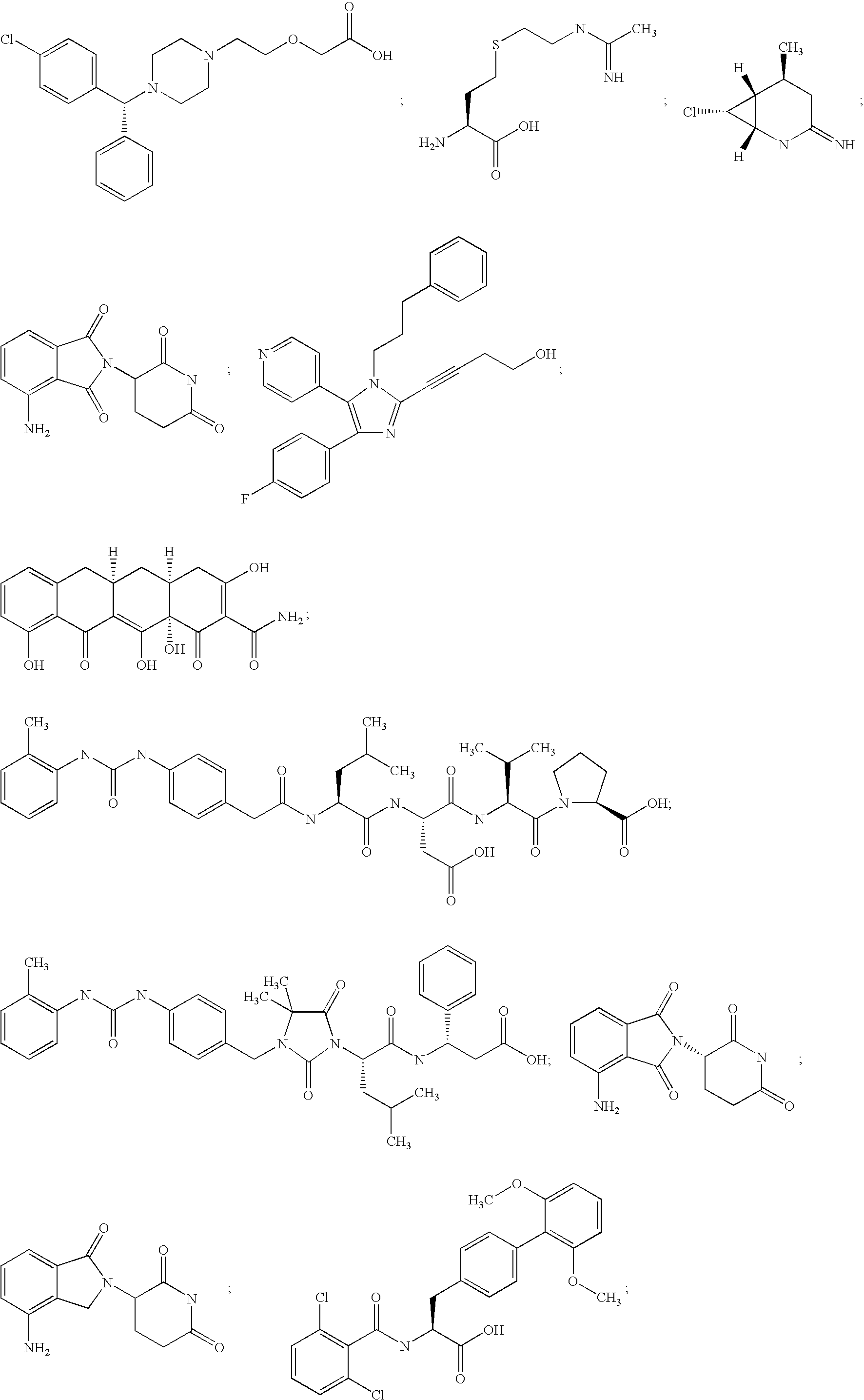

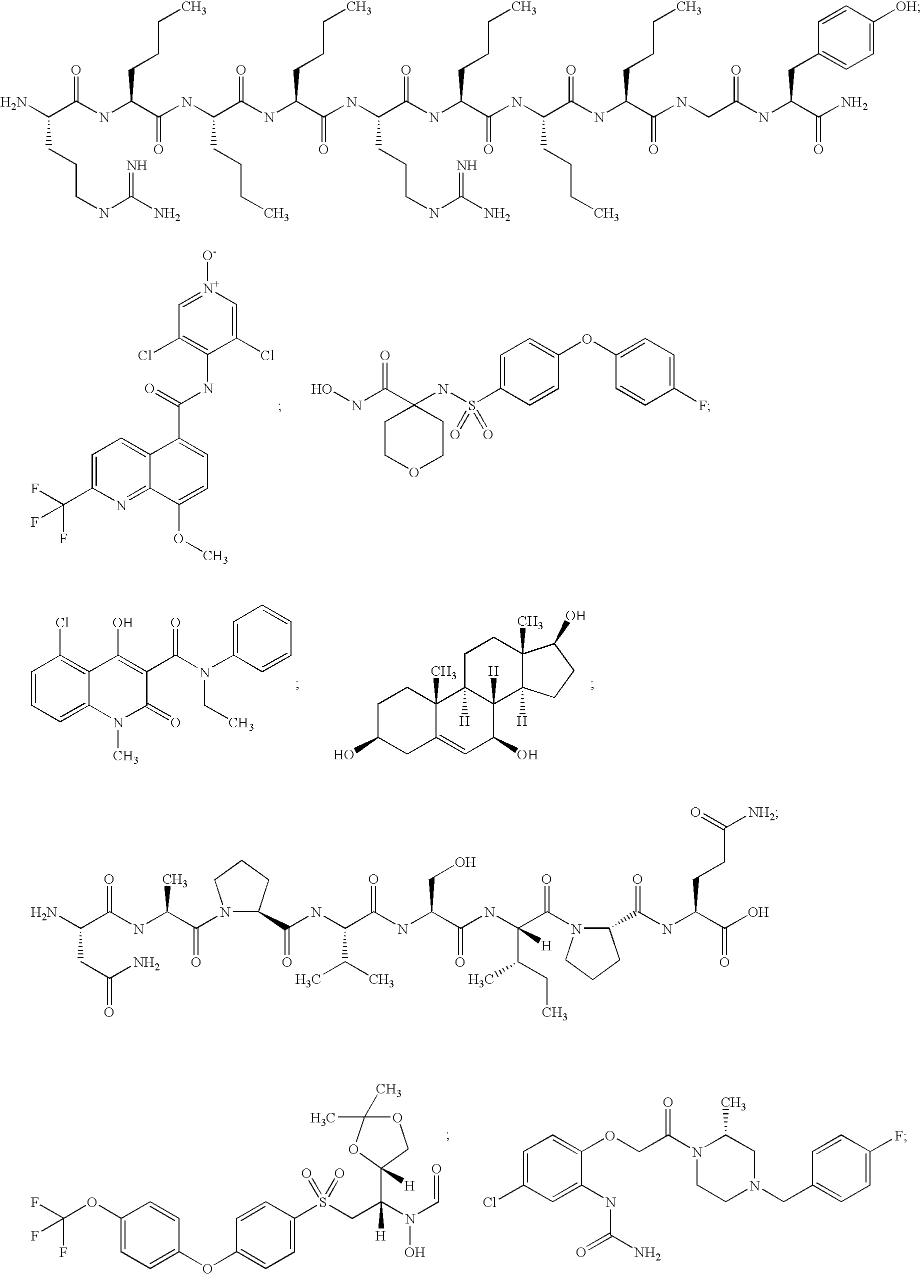

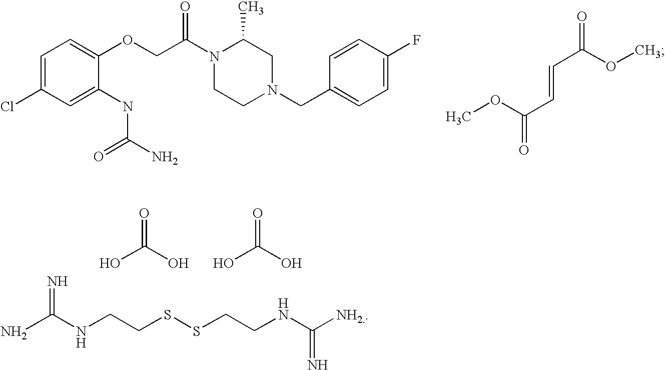

Further examples of pharmaceutical agents that can be co-administered with the compounds of formulas (I), (IA), and (IB) include:

T-cell receptor (TCR) Vβ6 CDR2 peptide vaccine consisting of TCR Vβ6, amino acid sequence 39-58, Leu-Gly-Gln-Gly-Pro-Glu-Phe-Leu-Thr-Tyr-Phe-Gln-Asn-Glu-Ala-Gln-Leu-Glu-Lys-Ser (SEQ ID NO:1);

Myelin basic protein immunogen peptide, aminoacid sequence 75-95, Lys-Ser-His-Gly-Arg-Thr-Gln-Asp-Glu-Asn-Pro-Val-Val-His-Phe-Phe-Lys-Asn-Ile-Val-Thr (SEQ ID NO:2);

Tiplimotide, myelin basic protein immunogen vaccine peptide, aminoacid sequence 83-99, D-Ala-lys-pro-val-val-his-leu-phe-ala-asp-ile-val-thr-pro-arg-thr-pro, (SEQ ID NO:3);

Myelin basic protein immunogen peptide, aminoacid sequence 82-98, Asp-glu-asp-pro-val-val-his-phe-phe-lys-asp-ile-val-thr-pro-arg-thr, (SEQ ID NO:4);

Adrenocorticotropic hormone (ACTH), Ser-Tyr-Ser-met-glu-his-phe-arg-try-gly-lys-pro-val-gly-lys-lys-arg-arg-pro-val-lys-val-tyr-pro-asp-gly-ala-glu-asp-glu-leu-ala-glu-ala-phe-pro-leu-glut-phe, (SEQ ID NO:5).

Further examples of pharmaceutically active agents that are effective against multiple sclerosis and are suitable to be administered in combination with compounds of the present invention include:

3-4 diaminopyridine; ABT-874; Actos® (pioglitazone); ALCAR (acetyl-L-carnitine); Alpha lipoic acid; AndroGel® (testosterone gel); combination of trimethoprim and vitamin C; combination of azithromycin and rifampin; minocycline; donezepil HCL; Avandia® (rosiglitazone maleate; combination of IFN beta-1a) and acetaminophen, ibuprofen or prednisone; combination of Avonex® (interferon beta-1a)+ CellCept® (mycophenolate mofetil); combination of Avonex® (interferon beta-1a) and Copaxone® (glatiramer acetate); combination of Avonex® (interferon beta-1a) and doxycycline; combination of Avonex® (interferon beta-1a) and EMLA (lidocaine and prilocalne) anesthetic cream; Avonex® (interferon beta-1a) and estrogen and progesterone; combination of Avonex® (interferon beta-1a)+Fludara® (fludarabine phosphate); combination of Avonex® (interferon beta-1a) and methotrexate and leucovorin rescue; combination of Avonex® (interferon beta-1a) and methotrexate and methylprednisolone; combination of Avonex® (interferon beta-1a) and Novantrone® (mitoxantrone); combination of Avonex® (interferon beta-1a) and Prozac® (fluoxetine); combination of Avonex® (interferon beta-1a) and Topamax® (topiramate); combination of Avonex® (interferon beta-1a) and Zocor® (simvastatin); AVP-923 (dextromethorphan/quinidine); combination of Betaseron® (interferon beta-1b) and Imuran® (azathioprine); combination of Betaseron® (interferon beta-1b) and Copaxone® (glatiramer acetate); combination of BHT-3009-01 and Lipitor® (atorvastatin); Bone marrow/peripheral stem cell transplant; CellCept® (mycophenolate mofetil); combination of CellCept® (mycophenolate mofetil) and Avonex® (interferon beta-1a); Oral cladribine; CNTO 1275 (monoclonal antibody); combination of Copaxone® (glatiramer acetate) and Antibiotic therapy (minocycline); combination of Copaxone® (glatiramer acetate) and Novantrone® (mitoxantrone); combination of Copaxone® (glatiramer acetate) and prednisone; combination of Copaxone® (glatiramer acetate) and Proventil® (albuterol); Cyclophosphamide; Daclizumab; Deskar® (pirfenidone); Estriol; Fumaric acid esters; Gabitril® (tiagabine HCL); Ginkgo biloba; IDEC-131 (anti-CD40L or anti-CD 154); the combination of Immunoglobulin and methylprednisolone; Inosine; Interferon tau; Lamictal® (lamotrigine); Lexapro® (escitalopram); Lipitor® (atorvastatin); combination of Lipitor® (atorvastatin) and Rebif® (interferon beta-1a); combination of Lymphocytapheresis (removal of immune cells), Imuran® (azathioprine) and prednisone; MBP8298; Methylprednisolone; combination of Methylprednisolone and Avonex (interferon beta-1a); Modiodal (modafinil); NBI-5788 (altered peptide ligand); combination of Novantrone® (mitoxantrone for injection concentrate) and Avonex® (Interferon beta-1a) or Copaxone® (glatiramer acetate); Omega-3 Fatty Acid Supplementation; Pixantrone (BBR 2778); combination of Provigil® (modafinil) and Avonex® (interferon beta-1a); Rapamune® (sirolimus); RG2077; Rituxan® (rituximab); Rolipram (phosphodiesterase-4 inhibitor); SAIK-MS (laquinimod, ABR-215062); T cell vaccination; Teriflunomide; Tetrahydrocannabinol; Tetrahydrocannabinol (dronabinol); Thalamic stimulation; combination of Tysabri® (natalizumab) and Avonex® (interferon beta-1a); combination of Tysabri® (natalizumab) and Copaxone® (glatiramer acetate); and Viagra® (sildafenil citrate).

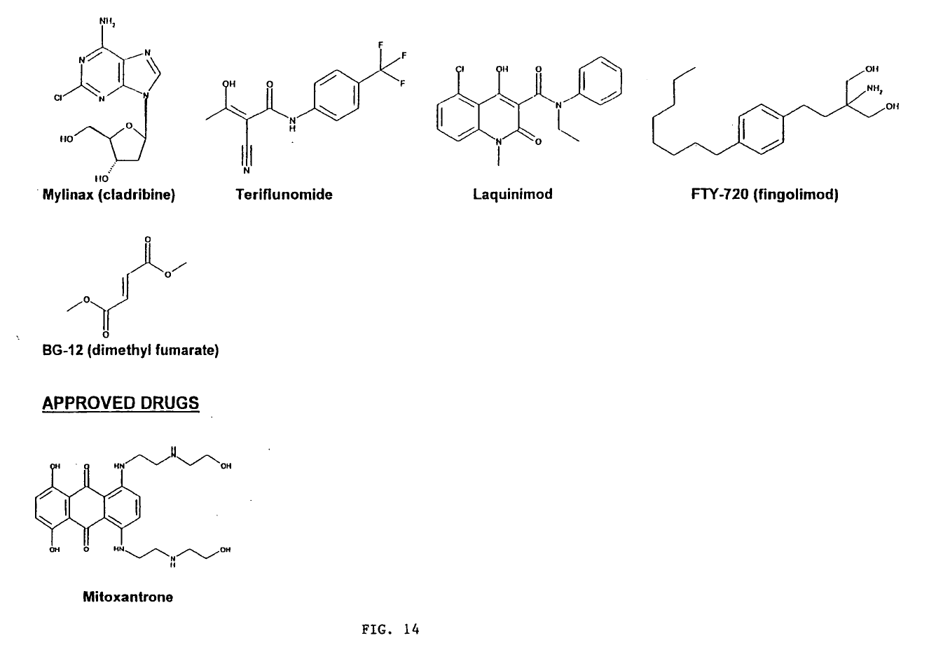

Further examples of pharmaceutically active agents that are effective against multiple sclerosis and are suitable to be administered in combination with compounds of the present invention include compounds listed in FIG. 14. Additionally, Copaxone (Glatiramer) can be orally co-administered with the compounds of the present invention.

In other embodiments, pharmaceutically active agents that are effective against multiple sclerosis and are suitable to be administered in combination with compounds of the present invention include compounds include: Mylinax, an oral formulation of cladrlbine used in leukaemia treatment, developed by Serono/Ivex; Teriflunomide, a metabolite of Arava, an oral immunosuppressant, developed by Sanofl-Aventis; FTY 720, an oral immunomodulator (Sphingosine-1-phosphate receptor agonist), developed by Novartis; MBP 8298, a synthetic myelin basis protein designed to reduce the emergence of antibodies directed against the myelin, developed by Bio MS Medical; an orphan drug 4-aminopyridline (4-AP), a potassium channel blocker, developed by Acorda; Gamunex, an intravenous immunoglobulin formulation, developed by Bayer; BG-12 fumarate, a second generation oral futnarate, developed by Biogen Idec/Fumapharm; Temsirolimus, a T-lymphocytes proliferation blocker, developed by Wyeth; E-2007, an AMPA receptor agonist, developed by Eisal; Campath, a humanized antibody directed against CD52, developed by Genzyme; Neuro Vax, a vaccine, developed by Immune Response; Zocor, a statin, developed by Merck; NBI 5788, a myelin-mimicking peptide ligand, developed by Neurocrine; Tauferon, Interferon tau, developed by Pepgen; Zenapax, a humanized anti-CD25 immunosuppressive antibody, developed by Protein Design; a combination of MS-IET and EMZ 701, a methyl donator, developed by Transition Therapeutics; Laquinlmod, an oral formulation of a derivative of linomide, developed by Active Biotech/Teva; deskar pirfenidone, a TNF-alpha inhibitor, developed by Mamac; ATL-1102, a second generation antisense inhibitor targeting VLA4, developed by Antisense Therapeutics.

In some embodiments, compounds of formula (A) can be administered in combination with antivascular agents, in particular agents inhibiting the growth factor receptors, Epidermal Growth Factor Receptor (EGFR), Vascular Epidermal Growth Factor Receptor (VEGFR), and Fibroblast Growth Factor Receptor (FGFR). Examples of such agents include, Iressa, Tarceva, Erbitux, Pelitinib, AEE-788, CP-547632, CP-547623, Tykerb (GW-2016), INCB-7839, ARRY-334543, BMS-599626, BIBW-2992, Falnidamol, AG1517, E-7080, KRN-951, GFKI-258, BAY-579352, CP-7055, CEP-5214, Sutent, Macugen, Nexavar, Neovastat, Vatalanib succinate, GW-78603413, Lucentis, Teavigo, AG-13958, AMG-706, Axitinib, ABT-869, Evizon, Aplidin, NM-3, PI-88, Coprexa, AZD-2171, XL-189, XL-880, XL-820, XL-647, ZK-CDK, VEGFTrap, OSI-930, Avastin, Revlimid, Endostar, Linomide, Xinlay, SU-668, BIBF-1 120, BMS-5826624, BMS-540215.

In some embodiments, compounds of formulas (I), (IA), and (IB) can be administered in combination with agents that affect T-cell homing, extravastion and transmigration. Examples of such agents include, FTY-720PKI-166, PTK-787, SU-11248.

In some embodiments, compounds of formulas (I), (IA), and (IB) can be administered in combination with agents inhibiting VLA-4. Examples of such agents include, Tysabri, Bio-1211. HMR-1031, SB-683698, RBx-4638,RO-0272441, RBx-7796,SB-683699, DW-908e, AJM-300, and PS-460644.

Daily dose of administration of the compounds of the present invention can be repeated, in one embodiment, for one week. In other embodiments, daily dose can be repeated for one month to six months; for six months to one year; for one year to five years; and for five years to ten years. In other embodiments, the length of the treatment by repeated administration is determined by a physician.

In another embodiment, the present invention is a method of treating a subject suffering from a cancer. As used herein, the term “cancer” refers to the uncontrolled growth of abnormal cells that have mutated from normal tissues. A cancerous tumor (malignancy) is of potentially unlimited growth and expands locally by invasion and systemically by metastasis. Examples of cancers that can be treated by the compounds of the present invention include: colorectal cancer, non-small cell lung cancer, ovarian, renal, sarcoma, melanoma, head & neck, hepatocellular, thyroid, multidrug-resistant leukemia, lymphoma, and multiple myeloma.

“Treating a subject suffering from cancer” includes achieving, partially or substantially, one or more of the following: arresting the growth or spread of a cancer, reducing the extent of a cancer (e.g., reducing size of a tumor or reducing the number of affected sites), inhibiting the growth rate of a cancer, and ameliorating or improving a clinical symptom or indicator associated with a cancer (such as tissue or serum components).

Treating, as used herein, refers to partially or totally inhibiting, delaying, or reducing (partially or completely) the progression of cancer including cancer metastasis; inhibiting, delaying or preventing the recurrence of cancer including cancer metastasis in, for example, a human.

In one embodiment the cancer selected from the group consisting of ovarian cancer, melanoma, renal cancer, sarcoma (e.g., liposarcoma and leiomyosarcoma), hepatocellular cancer, thyroid cancer, head and neck cancer, non-small cell lung cancer and colorectal cancer is a solid tumor. As used herein, a “solid tumor” is used to distinguish between a localized mass of tissue and leukemia. Leukemia is a cancer that starts in blood-forming cells of the bone marrow as a result of an abnormal development of leukocytes (white blood cells) and their precursors.

Different kinds of solid tumors are named for the type of cells of which they are composed and include but are not limited to, for example, sarcomas (cancers arising from connective or supporting tissues such as bone or muscle), carcinomas (cancers arising from the body's glandular cells and epithelial cells, which line body tissues) and lymphomas (cancers of the lymphoid organs such as the lymph nodes, spleen, and thymus, which produce and store infection-fighting cells).

As used herein the term “cancer” can be primary or secondary. Primary cancer is named after their original cell type, from the organ where the cancer first begins to grow. Cancer cells can break away from the primary site and travel to other parts of the body in the blood or lymphatic system. The cells eventually lodge in another body organ and begin to grow there. This is called a secondary cancer.

As used herein “ovarian cancer”, is cancer of the ovaries or fallopian tubes, including cancers of germ cells, stromal cells, and epithelial cells. Examples of ovarian cancers include but are not limited to:

Epithelial Ovarian Tumors, which include but are not limited to, serous adenomas, mucinous adenomas, and Brenner tumors, tumors of low malignant potential (LMP tumors), borderline epithelial ovarian cancer, epithelial ovarian cancers, carcinomas and undifferentiated epithelial ovarian carcinomas;

Germ Cell tumors which include but are not limited to, teratoma, dysgerminoma, endodermal sinus tumor, and choriocarcinoma; and

Stromal tumors, which include but are not limited to, granulosa cell tumors, granulosa-theca tumors, and Sertoli-Leydig cell tumors.

“Renal cancer” or “kidney cancer”, as used herein, includes but is not limited to, transitional cell cancer (TCC) of the renal pelvis, Wilms Tumour and renal cell cancer.

Renal cell cancer is also called renal adenocarcinoma or hypemephroma. In renal cell cancer, the cancerous cells are found in the lining of the tubules (the smallest tubes inside the nephrons that help filter the blood and make urine).

There are several types of renal cell cancer including but not limited to clear cell, chromophilic, chromophobic, oncocytic, collecting duct and sarcomatoid.

Renal cancer also includes cancers containing more than one of the cell types described above.

As used herein, “melanoma” is a type of skin cancer that occurs in the cells that color the skin, called melanocytes. Types of melanoma include but are not limited to:

Cutaneous melanoma, superficially spreading melanoma, nodular malignant melanoma, lentiginous malignant melanoma, acral lentiginous melanoma, demoplastic malignant melanomas, giant melanocytic nevus, amelanotic malignant melanoma, acral lentiginous melanoma unusual melanoma variants, including mucosal malignant melanoma and ocular malignant melanoma.

“Sarcomas”, as used herein, include but are not limited to, fibrosarcomas from fibrous body tissues, leiomyosarcomas and rhabdomyosarcomas from muscle tissues, liposarcomas from fat, synovial sarcomas, angiosarcomas from blood vessels, MPNST—malignant peripheral nerve sheath tumours (PNSTs), GIST—gastrointestinal stromal sarcoma, osteosarcoma, myosarcoma, chondrosarcoma, bile duct sarcoma, brain sarcoma, breast sarcoma, soft tissue sarcoma, uterine sarcoma, endocardial sarcoma, stromal sarcomas from supporting tissues (endometrial stromal sarcoma), granuloytic, histiolytic, hemangioendothelial, Kupffer-cell, neurogenic, round-cell, reticulum cell, spindle cell, Kaposi's sarcoma of the skin, Ewing's sarcomas and PNETs. In certain embodiments, the sarcoma is leiomyosarcoma or liposarcoma.

“Thyroid cancer”, as used herein, includes but is not limited to, papillary and/or mixed papillary/follicular, follicular and/or Hurthle cell, lymphoma, medullary, anaplastic and combinations thereof.

The term “head and neck cancer” as used herein, encompasses tumors that occur in several areas of the head and neck region, including the nasal passages, sinuses, mouth, throat, larynx (voice box), swallowing passages, salivary glands, and skin cancers that develop on the scalp, face, or neck may also be considered head and neck cancers. These cancers include but are not limited to squamous cell carcinoma, mucoepidermoid carcinoma, adenoid cystic carcinoma, lymphoma, adenocarcinoma, esthesioneuroblastoma, tumors of the nasal cavity and paranasal sinuses, nasopharyngeal cancer, cancers of the oral cavity (including all the various parts of the mouth: the lips; the lining inside the lips and cheeks (the buccal mucosa); the bottom of the mouth; the front of the tongue; the front part of the top of the mouth (the hard palate); the gums; and the area behind the wisdom teeth (the retromolar trigone)), tumors of the oropharynx, hypopharyngeal tumors, laryngeal cancer and salivary gland cancer (including malignant salivary gland tumor).

As used herein “hepatocellular cancer” or “liver cancer” includes but is not limited to: hepatocellular carcinoma (also sometimes called hepatoma or HCC) “carcinoma”, fibrolamellar HCC, cholangiocarcinoma, angiosarcoma (also be called haemangiosarcoma) and hepatoblastoma.

As used herein, “non-small cell lung cancer” includes, squamous cell carcinoma, adenocarcinoma and undifferentiated non-small cell lung cancer (undeveloped cancer cells are known as undifferentiated cells) and large cell carcinoma.

“Colorectal cancer” as used herein, includes any type of colon or rectal cancer, including but not limited to, adenoscarcinoma, sarcoma, melanoma, stromal, carcinoid, and lymphoma.

In one embodiment, the present invention is a method of treating a patient suffering from an acute myeloid leukemia characterized by a FLT3 mutation.

In certain embodiments, the compound of formulas (I), (IA), and (IB) can be administered in combination with an anti-cancer agent.

The compounds used in the present invention can be administered alone or in combination with one or more other pharmaceutically active agents that are effective against the cancer being treated.

As used herein, the term “combination” with reference to pharmaceutically active agents and the term “co-administering” and “co-administration” refer to administering more than one pharmaceutically active agent to a patient during one treatment cycle and not necessarily simultaneous or in a mixture.

Anti-cancer agents that can be employed in combination with the compounds of the invention include Taxol™ (also referred to as “paclitaxel”, and compounds that have the basic taxane skeleton), Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin and zorubicin hydrochloride.

Other anti-cancer drugs that can be employed in combination with the compounds described herein include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred anti-cancer drugs are 5-fluorouracil and leucovorin.

Other chemotherapeutic agents that can be employed in combination with the compounds of the invention include but are not limited to alkylating agents, antimetabolites, natural products, or hormones.

In the embodiments, in which the compounds of the invention are used to treat cancer, the dosage range at which the disclosed compounds, for example, compounds of formulas (I), (IA) and (IB), including the above-mentioned examples thereof, exhibit their ability to act therapeutically can vary depending upon the severity of the condition, the patient, the formulation, other underlying disease states that the patient is suffering from, and other medications that may be concurrently administered to the patient. Generally, the compounds described herein will exhibit their therapeutic activities at dosages of between about 0.1 mg/m² free base equivalent per square meter of body surface area/single dose to about 1000 mg/m² free base equivalent per square meter of body surface area/single dose. For example, the single dosage range can be between 10-800 mg/m², 100-700 mg/m², 400-600 mg/m², 420-550 mg/m² or 440-500 mg/m² or the single dose can be 480 mg/m².

In treating a patient afflicted with a conditions described above, all of the disclosed compounds can be administered in any form or mode which makes the compound bioavailable in therapeutically effective amounts. For example, compounds of formulas (I), (IA), and (IB) can be administered in a form of a pharmaceutically acceptable salt. The term “pharmaceutically acceptable salts” means either an acid addition salt or a basic addition salt, whichever is possible to make with the compounds of the present invention. “Pharmaceutically acceptable acid addition salt” is any non-toxic organic or inorganic acid addition salt of the base compounds represented by formulas (I), (IA), and (IB). Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tri-carboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicyclic, 2-phenoxybenzoic, p-toluenesulfonic acid and sulfonic acids such as methanesulfonic acid and 2-hydroxyethanesulfonic acid. Either the mono- or di-acid salts can be formed, and such salts can exist in either a hydrated or substantially anhydrous form. In general, the acid addition salts of these compounds are more soluble in water and various hydrophilic organic solvents and which in comparison to their free base forms, generally demonstrate higher melting points. “Pharmaceutically acceptable basic addition salts” means non-toxic organic or inorganic basic addition salts of the compounds of formulas (I), (IA), and (IB). Examples are alkali metal or alkaline-earth metal hydroxides such as sodium, potassium, calcium, magnesium or barium hydroxides; ammonia, and aliphatic, alicyclic, or aromatic organic amines such as methylamine, trimethylamine and picoline. The selection of the appropriate salt may be important so that the ester is not hydrolyzed. The selection criteria for the appropriate salt will be known to one skilled in the art.

Compounds of the present invention can be administered by a number of routes including orally, sublingually, buccally, subcutaneously, intramuscularly, intravenously, transdermally, intranasally, rectally, topically, and the like. One skilled in the art of preparing formulations can determine the proper form and mode of administration depending upon the particular characteristics of the compound selected for the condition or disease to be treated, the stage of the disease, the condition of the patient and other relevant circumstances. For example, see Remington's Pharmaceutical Sciences, 18^{th} Edition, Mack Publishing Co. (1990), incorporated herein by reference.

The compound of formulas (I), (IA), and (IB) of this invention may also be administered topically, and when done so the carrier may suitably comprise a solution, ointment or gel base. The base, for example, may comprise one or more of petrolatum, lanolin, polyethylene glycols, bee wax, mineral oil, diluents such as water and alcohol, and emulsifiers and stabilizers.

The solutions or suspensions may also include one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampules, disposable syringes or multiple dose vials.

The invention is illustrated by the following examples, which are not intended to be limiting in any way.

### EXEMPLIFICATION

### Example 1

### Symadex™ Inhibits Proliferation of B-Cells Following Stimulation with LPS and T-Cells Following Stimulation with Con A in In Vitro Experiments

The activity of Symadex™ was compared to mitoxantrone in several in vitro assays to determine the effect of Symadex™ on several key regulatory systems involved in multiple sclerosis neuroinflammation and antigen presentation.

IL-4 serves as a growth and differentiation factor for B cells, mast cells and macrophages and is a switch factor for synthesis of IgE in mice. It also promotes growth of a cloned CD4⁺ T cell and enhances class II MHC molecule expression and resting B lymphocytes enlargement. In man, CD4⁺ T lymphocytes also produce IL-4, but the human variety has not been shown to serve as a B cell or mast cell growth factor. Both murine and human IL-4 induce switching of B lymphocytes to synthesize IgE. Human IL-4 also induces CD23 expression by B lymphocytes and macrophages in man. IL-4 may have some role in cell mediated immunity.

IL-10 inhibits cytokine synthesis by T_{H}1 cells, blocks antigen presentation, and inhibits the formation of interferon γ. IL-10 inhibits the macophage's ability to present antigen and to form IL-1, IL-6 and TNF-α. IL-10 also participates in IgE regulation. Although IL-10 suppresses cell-mediated immunity, it stimulates B lymphocytes, IL-2 and IL-4 T lymphocyte responsiveness in vitro, and murine mast cells exposed to IL-3 and IL-4. IL-10 may find therapeutic utility by suppressing T lymphocyte autoimmunity in multiple sclerosis and type I diabetes mellitus as well as in facilitating allograft survival.

In this experiment, test compound and/or vehicle were preincubated with human peripheral blood mononuclear leukocyte (PBML, 1×10⁶/ml) in RPMI buffer pH 7.4 for 2 hours. Concanavalin A (Con A, 20 μg/ml) was then added to stimulate the cells overnight in 5% CO₂ at 37° C. IL-4 and IL-10 cytokine levels in the conditioned medium were then quantified using a sandwich ELISA kit. Compounds were screened at 10, 1, 0.1, 0.01 and 0.001 μM.

B-lymphocyte cells isolated from the spleen of balb/c mice weighing 17±1 g were used. Test compound and/or vehicle were incubated with the cells (1.5×10⁶/ml) in the presence of 10 μg/ml lipopolysaccharide (LPS) in AIM-V medium pH 7.4 at 37° C. for 24 hours. [³H]Thymidine (120 nM) was then added for an additional overnight incubation period. Thymidine incorporation was assessed by liquid scintillation counting.

T-lymphocyte cells isolated from thymus of balb/c mice weighing 17±1 g were used. Test compound and/or vehicle is incubated with the cells (4×10⁶/ml) in the presence of 3 μg/mL Concanavalin A (Con A) in AIM-V medium pH 7.4 at 37° C. for 24 hours. [³H]Thymidine (120 nM) was then added for an additional overnight incubation period. Thymidine incorporation was assessed by liquid scintillation counting. Compounds were screened at 10, 1, 0.1, 0.01 and 0.001 μM.

The results for mitoxantrone and Symadex™ are presented in Table 1. Test compound-induced suppression of cell proliferation by 50 percent or more (≧50%) relative to vehicle control response indicates significant inhibitory activity.

TABLE 1

| | | SYMADEX ™ | | Mitoxantrone | |
|---|---|---|---|---|---|
| | | % Growth | | % Growth | |
| | Conc. | Inhibition | IC50 | Inhibition | IC50 |
| Mediator | 10 μM | 105 | 3.33 μM | 108 | 2.96 μM |
| release, IL-4 | 1 μM | −9 | | −4 | |
| | 0.1 μM | 2 | | 18 | |
| | 10 nM | 7 | | 13 | |
| | 1 nM | 8 | | 11 | |
| Mediator | 10 μM | 99 | 1.2 μM | 102 | 0.759 μM |
| release, IL-10 | 1 μM | 39 | | 60 | |
| | 0.1 μM | 16 | | 5 | |
| | 10 nM | 16 | | 1 | |
| | 1 nM | 1 | | 2 | |
| Cell | 10 μM | 103 | 0.038 μM | 102 | 7.31 nM |
| Proliferation, | 1 μM | 101 | | 102 | |
| B-Cell + LPS | 0.1 μM | 80 | | 93 | |
| | 10 nM | 38 | | 54 | |
| | 1 nM | 0 | | 18 | |
| Cell | 10 μM | 104 | 0.014 μM | 103 | 0.032 μM |
| Proliferation, | 1 μM | 103 | | 103 | |
| T-Cell + | 0.1 μM | 82 | | 80 | |
| Con A | 10 nM | 44 | | 19 | |
| | 1 nM | 10 | | −1 | |

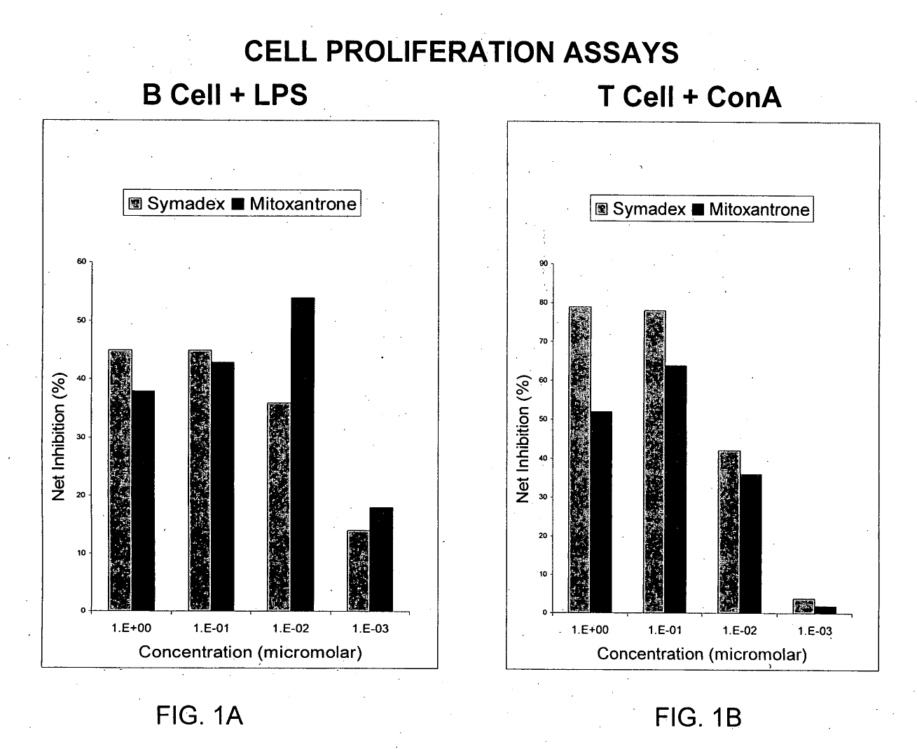

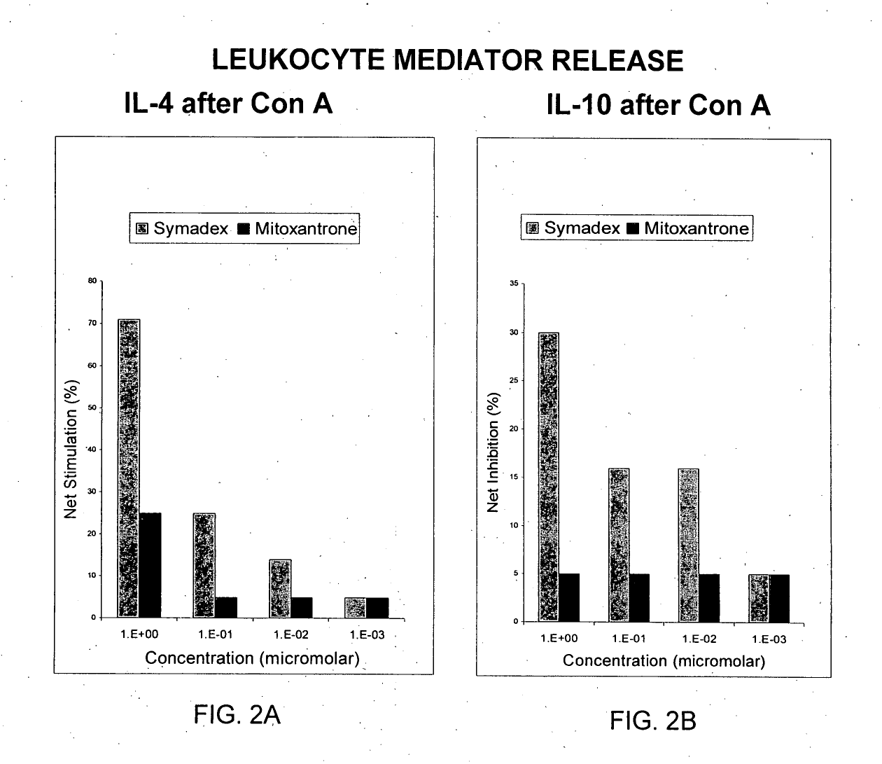

The results presented in Table 1, FIGS. 1A and 1B as well as in FIGS. 2A and 2B indicate that Symadex™ inhibits the release of inflammatory mediators IL-4 and IL-10, both of high importance in neuroinflammatory diseases such as multiple sclerosis. Furthermore, Symadex™ exhibited high level of growth inhibition in in vitro proliferation assays involving B- and T-cells. The activity of Symadex™ was comparable to that of the control compound, mitoxantrone.

### Example 2

### Symadex™ Alleviates the Symptoms of Experimental Autoimmune Encephalomyelitis (EAE), an Animal Model of Chronic Multiple Sclerosis in a Weekly Treatment Cycle for 4 Weeks

One Method of Showing the Utility of the a Pharmaceutical compound for the treatment of various conditions associated with multiple sclerosis (MS) is its ability to inhibit effects of Experimental Autoimmune Encephalomyelitis in laboratory animals.

Experimental Autoimmune Encephalomyelitis (EAE) is an animal model for MS, which entails inducing a T-cell-mediated autoimmune disease against myelin basic protein in certain susceptible mammalian species. The EAE model is an appropriate method for studying the inflammation of the brain and spinal cord associated with MS (see Bolton, C. Mult, Scler, 1995; 1(3); 143-9).

In rodents, injection of whole spinal cord or spinal cord components such as myelin basic protein induces an autoimmune response based on the activation of T-lymphocytes. Clinical disease typically becomes manifest around day 8-10 after inoculation, observed as a broad spectrum of behavioral anomalies ranging from mild gait disturbances and tail atony to complete paralysis and death. Weight loss typically occurs. In animals that survive, spontaneous recovery occurs, accompanied by variable recovery of most motor function. Depending on the species, allergen, and methodology used, animals tested by the EAE model may experience a single (acute EAE) or several (chronic relapsing EAE) attacks.

Treatments of EAE come in many structural forms: treatment can be prophylactic or preventative, whereby the therapeutic composition is administered before immunization; treatment can be initiated during the first week of induction; and treatment can be interventions, initiated after clinical symptoms are extent (acute or chronic). Prevention protocols are very common in the literature, treatment after disease is rarer, and treatment after weeks of disease are the most infrequent. The experiments reported herein are in the last classification in which animals in the chronic-progressive (CP) phase with extensive demyelinated plaques are treated. CP-EAE induced by whole CNS in complete Freund's adjuvant is a florid disease with extensive inflammatory and demyelinated changes. As a general philosophy, we believe that successful intervention at later times can better predict effectiveness in the human condition. This is particular relevant to the case of prevention studies, which concentrates on the peripheral immune system, rather than addressing the issue of existing CNS inflammation that is a characteristic of MS.

Methodology

In the present experiment, female juvenile Hartley guinea pigs (225 g) were immunized with homogenized whole CNS (in saline) with an equal amount of complete Freund's Adjuvant and 10 mg added killed M. tuberculosis. The animals (>95%) show clinical signs starting on day 7 post immunization. An acute event of varying severity occurs between day 7 and day 20 followed by a continuous accumulation of clinical abnormality with hind limb paralysis, fecal impaction and incontinence. Table 2 shows the clinical scoring scale. These clinical features indicate inflammation-induced lumbar spinal cord demyelination. A recent survey of previous experiments indicates that taking an animal past day 40, which has a clinical score of “2” for more than 1 week yields a 97% occurrence of demyelinated plaques in the cord.

In these experiments, the immunized animals were nursed until day 40 or day 52 and then treated with 8 mg/kg and 16 mg/kg Symadex™ (intra cardiac), or 20 mg/kg and 40 mg/kg Symadex™ (i.p.) once a week for 4 weeks. Controls were given vehicle. Clinical signs were scored daily and the weights recorded. At the completion of the treatment period, the brain and spinal cord were dissected, formalin fixed and blocked for routine pathological examination of meningeal inflammation, perivascular infiltration (cuffing), parenchymal myelitis and demyelination by a blinded observer using hematoxylin-eosin and solochrome R cyanin stained sections.

Untreated, chronic EAE animals (n=5) were sacrificed on day 40, as well as non-EAE controls (n=5). Following each 10-day treatment interval, five animals from each group were sacrificed (0.25 ml sodium pentobarbital), blood samples were collected for FACS analysis (see below), and the brain and spinal cord dissected and sectioned. Three spinal sections were used, corresponding to lumbar, thoracic and cervical regions of the cord. The brain was cut into five transverse sections; the first three proximal sections were combined in one block, and the last two distal sections in another. Tissues were fixed in 10% formalin and embedded in paraffin. Five micrometer sections were stained with hematoxylin-eosin (H-E) or solochrome-R-cyanin (SCR) and evaluated by a blinded observer in each of the four categories: meningeal inflammation, perivascular infiltration, encephalitis or myelitis and demyelination (Table 2). The combined pathological score represents the total score (out of a potential 20) from all five CNS sections in each animal.

TABLE 2
Pathological scoring scale

| M: | Inflammatory reaction in the meninges |
|---|---|
| 0: | no changes |
| 1: | perivascular and/or meningeal infiltration by mononuclear cells, 1-3 |
| | vessels involved |
| 2: | 4-6 vessels involved |
| 3: | 6+ vessels involved |
| 4: | dense infiltration of meninges with nearly all or all blood vessels |
| | involved |
| P: | Parenchymal perivascular infiltrations |
| 0: | no changes |
| 1: | 1-3 parenchymal vessels infiltrated in Virchow-Robin spaces |
| 2: | 4-6 vessels involved |
| 3: | 6+ vessels involved |
| 4: | virtually all vessels involved |
| E: | Encephalitis or myelitis |
| 0: | no invasion of the neural parenchyma; microglial or inflammatory |
| | cells invading neural parenchyma |
| 1: | a few scattered cells |
| 2: | invasion by cells from several perivascular cuffs |
| 3: | large areas of neural parenchyma involved |
| 4: | virtually the entire section is infiltrated |
| D: | Demyelination, remyelination and myelin debris |
| 0: | no demyelination |
| 1: | single focus on subpial demylination or myelin debris |
| 2: | several small foci of demyelination |
| 3: | one large confluent area of demyelination |
| 4: | several large confluent areas of demyelination |

To quantify the abnormalities observed in the spinal cord, sections stained with H-E were divided into 12 representative pie-shaped areas. In each area, the number of cells within a 0.12-mm² field of view was counted using Sigma Scan Pro image analysis software (SPSS), and the combined mean number of cell in all 12 areas was calculated for the whole spinal cord (36 fields of view per animal). Note that as all cell nuclei were counted, the number of cells may include neurons and glial cells in addition to infiltrates. Hence, the cell count in non-EAE animals served as a baseline.

Results

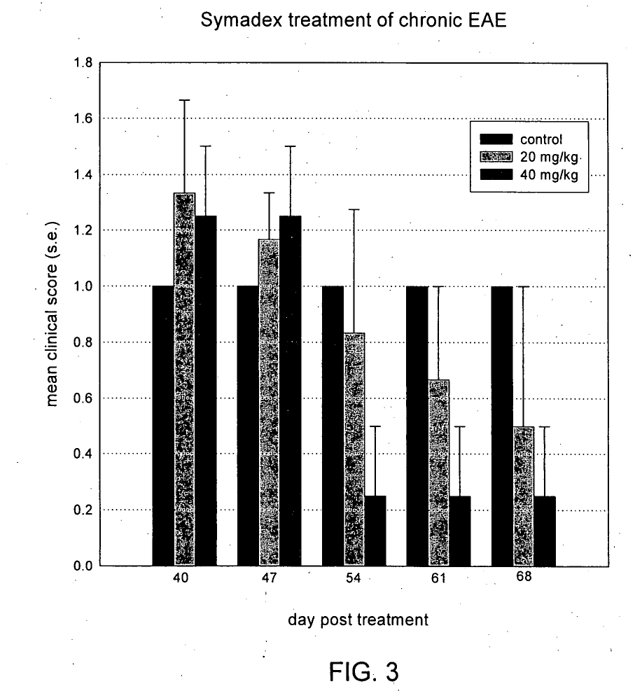

Symadex™ produced a profound and substantial change in the clinical progress and pathological findings when given at 20 and 40 mg/kg (i.p.). FIG. 3 illustrates the mean clinical score of the animals at the indicated day thereafter. The treated animals all showed some degree of clinical recovery with the 40 mg/kg group reaching recovery within 2 weeks of the start of treatment.

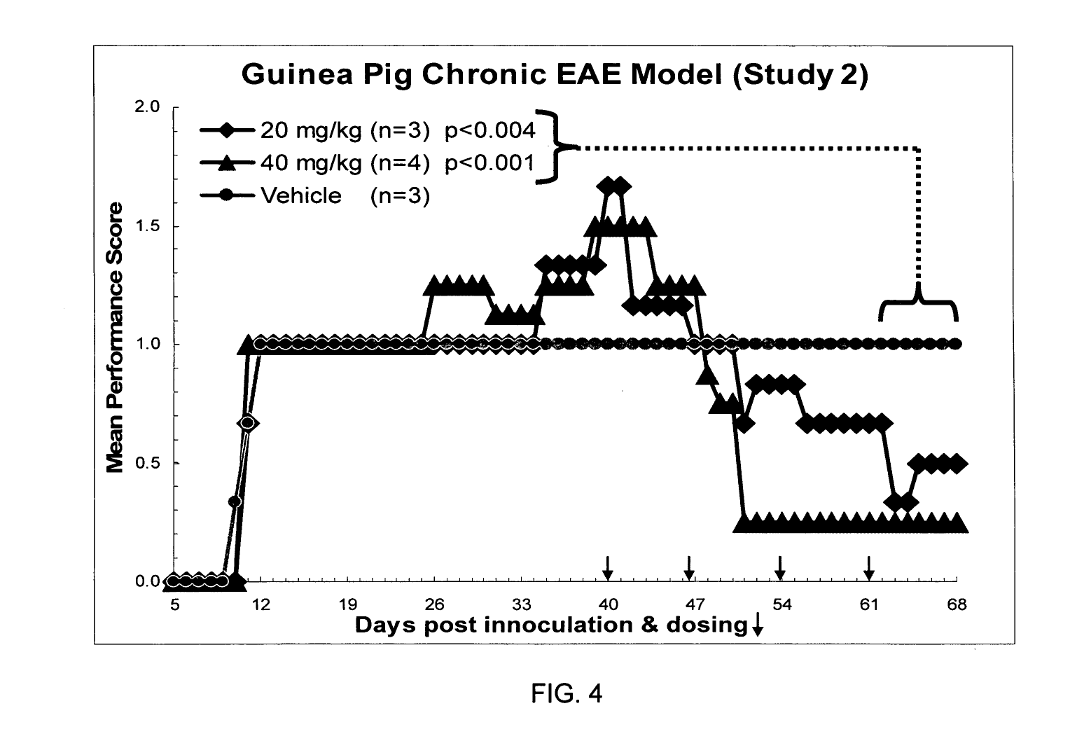

The longitudinal course of recovery from disease is further illustrated in FIG. 4. In this experiment, the vehicle controls showed a steady course of disease. Treated animals, in both the 20 and 40 mg/kg cohorts entered the study presenting disease of greater severity than controls, a coincidental circumstance attributed to the phenomenology of randomization prior to assignment of a treating group. As indicated by the arrows, treatment began on day forty and progressed for a total of four doses. At the end of the treatment, both treated cohorts showed significant disease improvement to levels significantly below control, despite having entered the study at a disease level well above control. Statistical significance, even with 3 and 4 animal small cohorts showed p values supporting the hypothesis that treatment afforded a statistically different outcome over control. These were determined by non-parametric comparisons by the Mann-Whitney or Wilcoxon rank-sum test for difference in medians. The significance values were 0.001 and 0.004 for the 40 and 20 mg/kg cohorts, respectively.

The pathological findings were most unusual. The scores for meningeal inflammation and perivascular infiltration were more severe in the treated groups than in vehicle controls (data not shown). However, we observed two highly significant findings: existing lesions had a profound loss of cells (data not shown) and we observed myelin pallor previously attributed to remyelination (data not shown). The latter observation is consistent with permissive remyelination of the CNS due to removal of the inflammatory cells.

Demyelination is a key pathological feature of the MS lesion. Not only does this alter electrical response of the axon, current thought suggests that prolonged demyelination can result in permanent axonal damage and death. Thus neuro-degeneration is also a key component of the MS pathological milieu. In this regard, Symadex™ has proved to be effective in permitting endogenous remyelination even after a period of disease progression that reached 97% spinal chord demyelination in this chronic-progressive model. It appears to permit this CNS recovery by reducing the inflammation in existing lesions. After prolonged Symadex™ treatment, it is possible to observe chronic demyelinated plaques that have virtually no remaining inflammatory cells and some of these lesions show the myelin pallor indicative of remyelination (called a shadow plaque in MS). Prevention of new T-cell infiltration by deletion of these cells or down regulation or inhibiting cell trafficking would prevent the recruitment of further macrophages to an inflammatory lesion. As a consequence, the immune cells in the lesions die by apoptosis and the lesions are left relatively free of infiltrates. Removal of the cytokine, and ROS-mediated tissue toxicity of macrophages would allow the CNS reparative mechanisms to become active and remyelination is observed. It is thus likely that Symadex™ has an effect on the peripheral immune system, although a direct effect on CNS inflammation cannot be ruled out.

The continued presence of large inflammatory cuffs and meningeal inflammation scores that were higher than control is consistent with the continued production of immune competent leukocytes which accumulate around CNS vessels, but do not traffic into the parenchyma.

### Example 3

### Symadex™ Alleviates the Symptoms of Experimental Autoimmune Encephalomyelitis (EAE), an Animal Model of Chronic Multiple Sclerosis in a Weekly Treatment Cycle for 4, 6, 8 and in a 4 Week on Drug-4 Week Off Treatment Cycle

The experiment described in Example 2 was extended to a larger cohort and longer treatment cycle with several objectives in mind. In addition to corroborating the initial findings, a concerted effort was directed at also demonstrating the extent and durability of response, including the effect after drug withdrawal, and to document the impact of drug treatment on immune function in order to uncover any signals of impending impairment or toxicity.

Following disease induction, as previously described, the animals were randomized into 5 five cohorts, one vehicle control and 4 treatment cohorts. Animals in the treatment cohorts were administered study drug intraperitoneally at 20 mg/kg (Symadex™ dihydrochloride trihydrate) once a week for 4, 6 and 8 weeks, with an additional cohort treated once a week for 4 weeks and observed for an additional 4 weeks of treatment with vehicle solution (saline) rather than with drug.

The only significant protocol deviation from the method of Example 2 was that the pool of immunized animals was culled of animals presenting with a disease severity greater than 2 and randomized so that the mean disease severity of each cohort was matched in the severity score range of 1 to 1.5. This measure was invoked in order to avoid the chance circumstance, observed in Example 2, that animal selected for treatment should start treatment with a more severe presentation than the corresponding vehicle controls.

All treated animals showed statistically significant improvement in disease. That is, their symptoms of paralysis attributable to the demyelinating progression of inflammatory cell assault on nerve chord parenchyma, were reduced close to baseline, pre-disease levels. These results are evident by mere inspection of the disease course plots and also proved to be highly significant by non-parametric, rank-order statistical analysis.

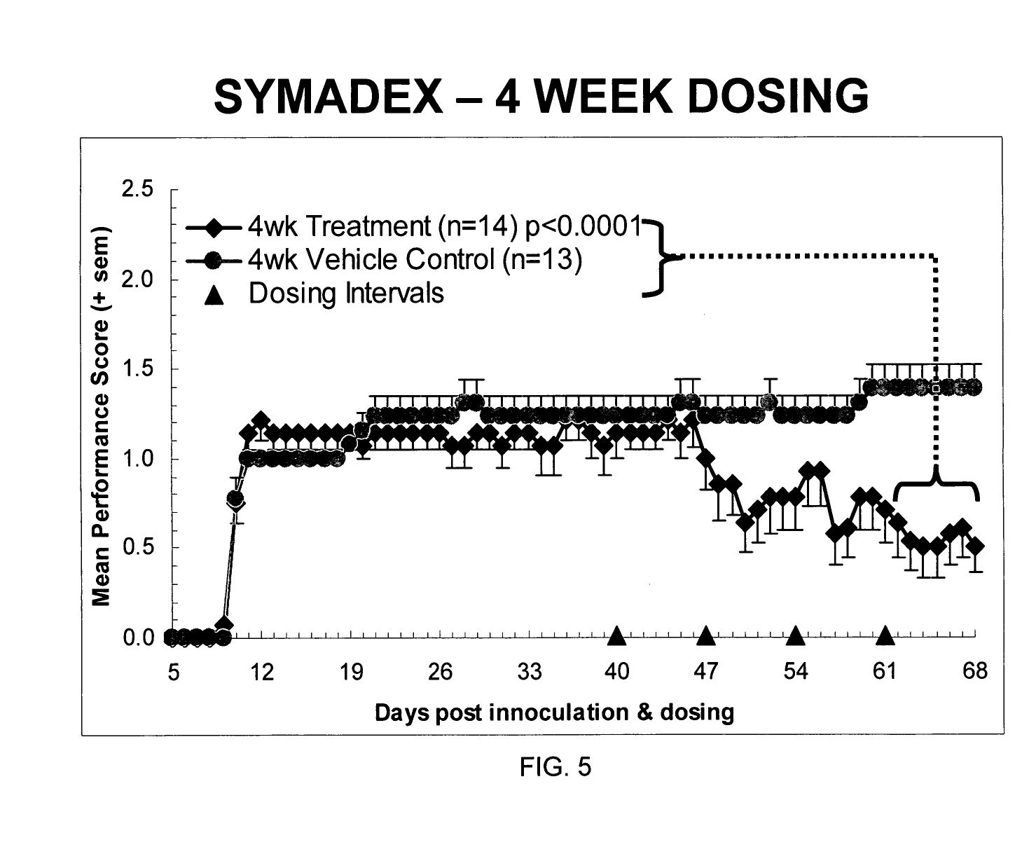

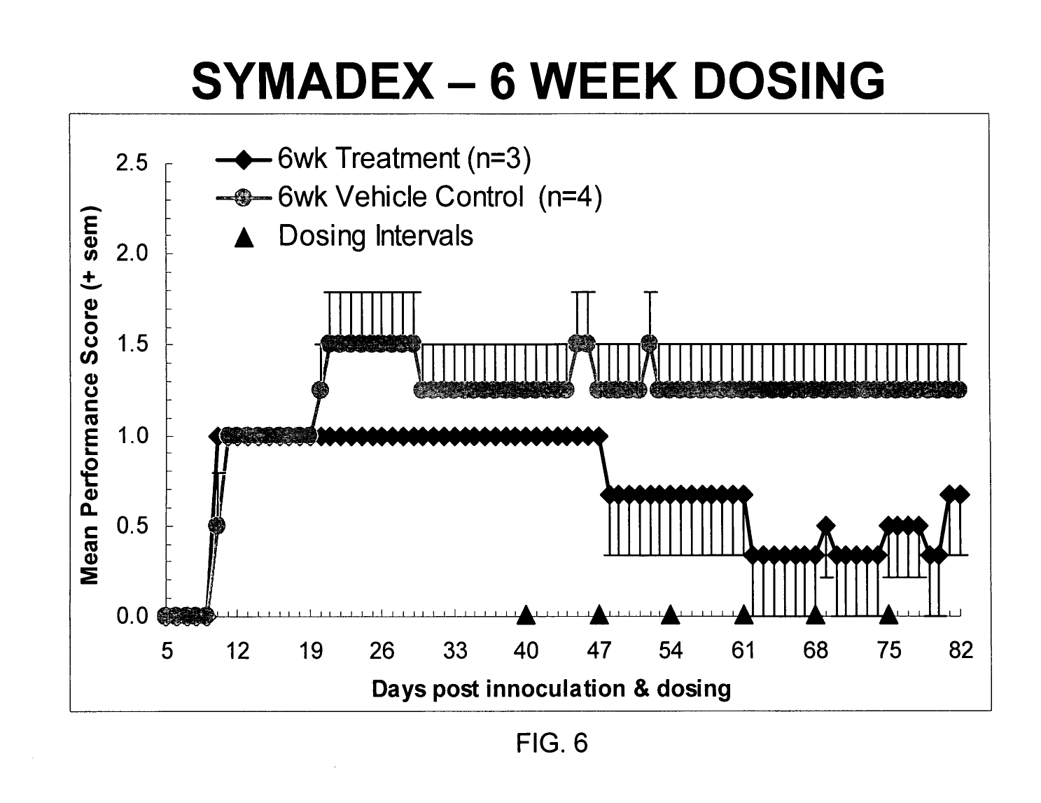

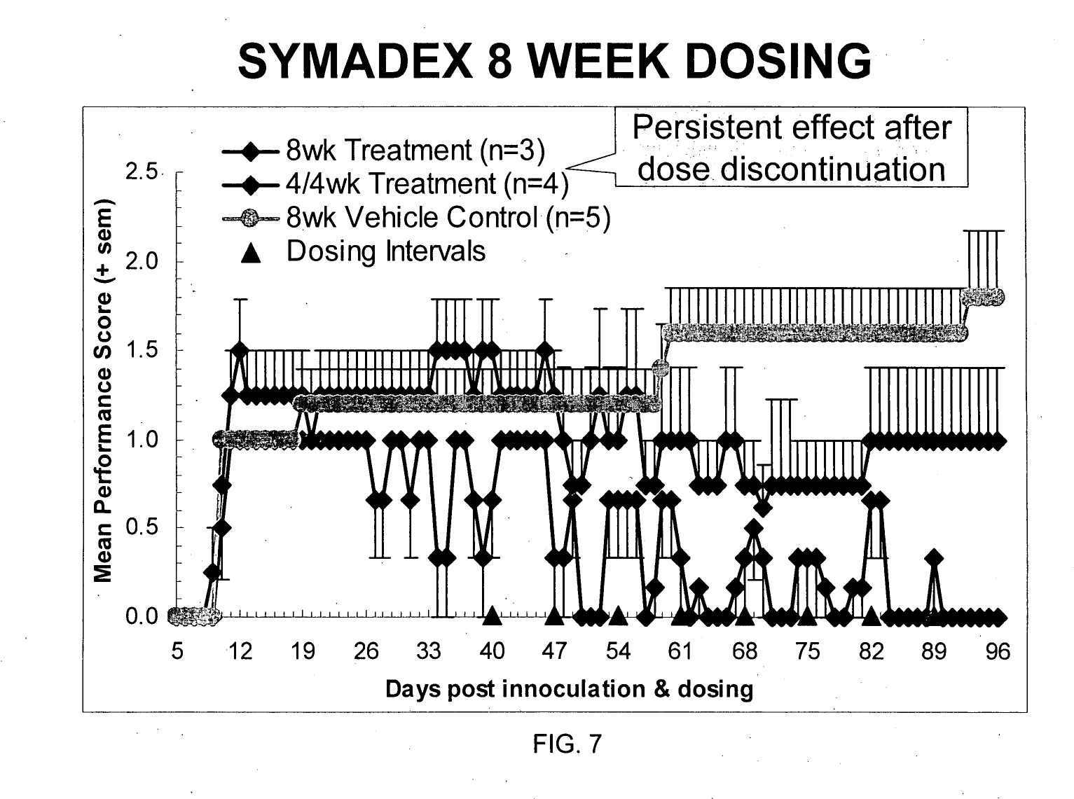

The 4-week treatment cycle result (n=14), as shown in FIG. 5, demonstrates a declining trend in clinical severity score to a mean level 0.7 from a starting disease presentation mean of 1.3. This change is statistically different from control (n=13), with a p value on differences in median of 0.0001. A similar dose response is demonstrated in FIG. 6, which describes the 6 week treatment course. Again, disease improved from a mean severity score of 1.3 for control (n=4) down to 0.3 (n=3) for the treatment cohort, with a statistical significance p value of 0.009. FIG. 7 presents the 8 week treatment cohorts. Attention is called to the vehicle control, whose disease shows progression towards greater severity, and hence greater paralysis attributable to demyelination, with a gradual rise after 4 weeks from a level of 1.3 to 1.7. In contrast, both treatment cohorts, show progressive disease amelioration, indication reversal of demyelination. In the case of animals treated with 8 consecutive doses, the trend towards normal, pre-disease clinical scores is initially variable but converges on baseline at the end of the treatment course. Despite the interindividual heterogeneity in recovery profile, the full rank-order analysis shows the difference between treatment (n=3) and control (n=5) to be significantly at a p value of 0.0006. The cohort with treatment discontinued treatment after 4 weeks, remained in stable disease at the time of discontinuation, also at a level significantly different from control, with a p value of 0.002.

The results of this study are shown in FIG. 7. First, the therapeutic response shows a graded temporal response against control. Sick animals become progressively healthier in proportion to the weekly duration of response, while control animals progress irreversibly towards complete neurological dysfunction, whose root cause is irreversible demyelination. Second, the cumulative pharmacodynamic effect of drug treatment is durable, because treated animals remain in stable condition, commensurate with their degree of treatment, while untreated controls sustain an accelerating progression in disease. This is a particularly noteworthy observation in light of the effects obtained with the most promising recent therapy for progressive MS, namely the α4 integrin antagonists like Tysabri and its small molecule ligand equivalents, as described by Piraino P. S. et al, J Neuroimmunology, 131:147-159, 2002). When treatment is discontinued in the same guinea pig EAE model of MS, the animals in the treatment cohort revert within 7 days to the disease level of untreated controls, with no evidence of a protracted beneficial pharmacodynamic effect.

The contrasting result between the therapeutic effects of Symadex and the α4 integrin antagonists, as well as between Symadex and other therapies that interdict T-cell mediated inflammatory responses, is that Symadex does not exert its action via the activation and recruitment of inflammatory cells. The histopathology of spinal chord from animals sacrificed at periodic interval throughout the time course of disease recovery show accumulation, rather than diminution, of inflammatory cells in blood vessels and perivascular cuffs, as noted in Example 2. Yet, these cells are apparently blocked from transmigrating beyond the basement membrane of parenchyma, suggesting a block via mechanisms that could involve: cell adhesion, motility, and extracellular matrix remodeling.

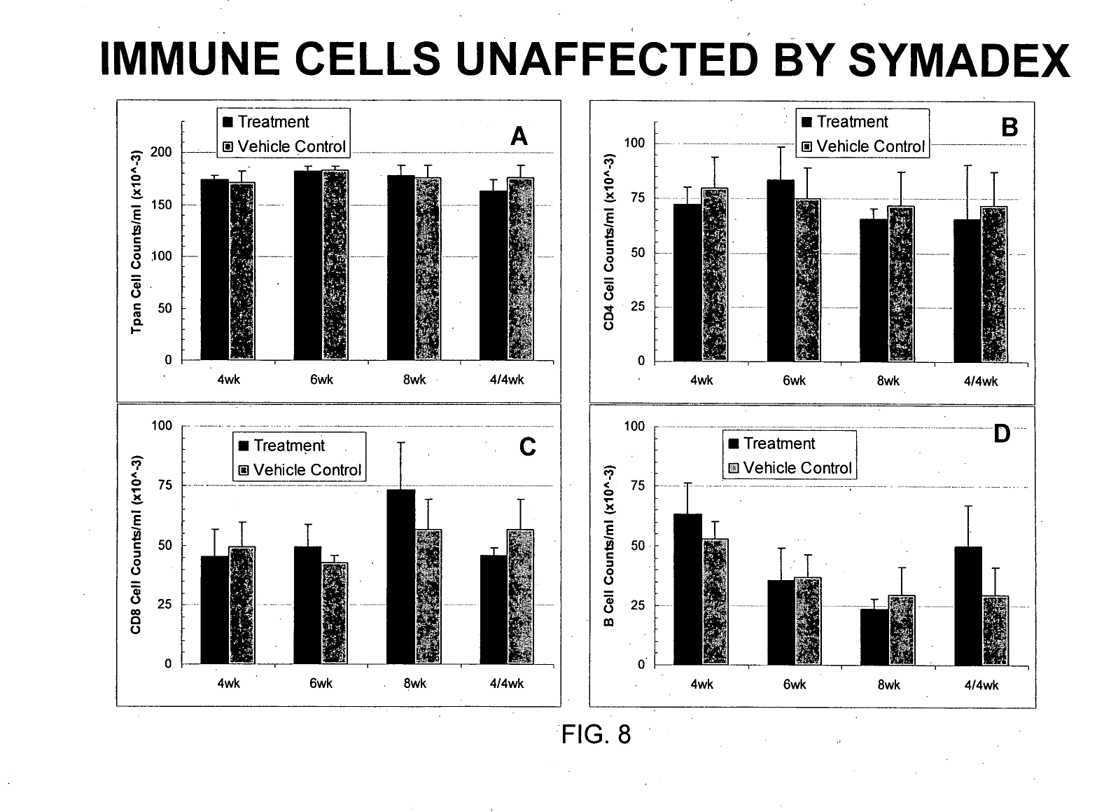

It is demonstrable as a differentiating, and unexpected, feature of Symadex's mode of action, when compared to corticosteroid, interferon, and integrin antagonist therapies that there are no changes in T-cell populations or in T-cell sub-type ratios. In the instant example, as shown in FIG. 8, Panel A, no difference is observed between any treatment cohort and vehicle control with respect to total T-cell counts. Neither are CD4 or CD8 T-cell populations modulated by Symadex™ treatment, as shown in panels B and C. Even B-cells show no significant deviation from controls, although there appears to be a declining trend across the board as a function of disease or treatment duration. Like the cytotoxic therapies for MS, e.g. mitoxantrone and cyclophosphamide, for example, Symadex™ may exert a transient diminution in B-cell counts in stimulated cell cultures, according to the evidence presented in Example 1, but the effect is not evident on prolonged exposure in vivo. This observation further reinforces the notion that Symadex™ acts by a novel mechanism that will not deplete the immune system nor diminish host defenses against antigen presenting pathogens. Such a property would be highly desirable and advantageous in any therapy intended for the treatment of chronic conditions or acute conditions, such as MS flair ups, in otherwise healthy subjects.

### Example 4

### Symadex™ Reverses the Symptoms of Experimental Autoimmune Encephalomyelitis (EAE), an Animal Model of Chronic Multiple Sclerosis After Two Doses Administered 72 Hours Apart

Analysis of the time course of disease recovery upon treatment with Symadex™ on a weekly basis reveals a two to three day periodicity in the amelioration of disease. This phenomenon is particularly evident in the 8 week treatment test cohort shown in FIG. 7. Individual animals in the cohort can be seen to respond differently so that the pharmacodynamic response on average presents itself in a “saw-tooth” pattern between successive dosing intervals. In tracing the records of specific animals within the cohort, it appears that some recover transiently and show disease improvement for 2-3 days after receiving a dose and then revert to a higher disease score. The overall trend leads to disease improvement over 8 weeks, but the “saw-tooth” response phenomenon raises questions about the temporal spacing of treatments that optimally achieve a smoother reversal of disease symptoms.

In order to test the possibility that a more frequent dosing schedule would offer a more rapid resolution of disease symptoms, an experiment was performed to match the dosing cycle to the observed periodicity of response. Accordingly, the method of Experiment 2 was applied to a cohort of animals and controls, which were allowed to reach the chronic phase of disease at 30 days post immunization. Six animals with a disease score of 1 were selected and half were treated with 20 mg/kg Symadex™ administered intraperitoneally. Two dose were given 72 hours apart to 3 animals. Three animals served as vehicle controls.

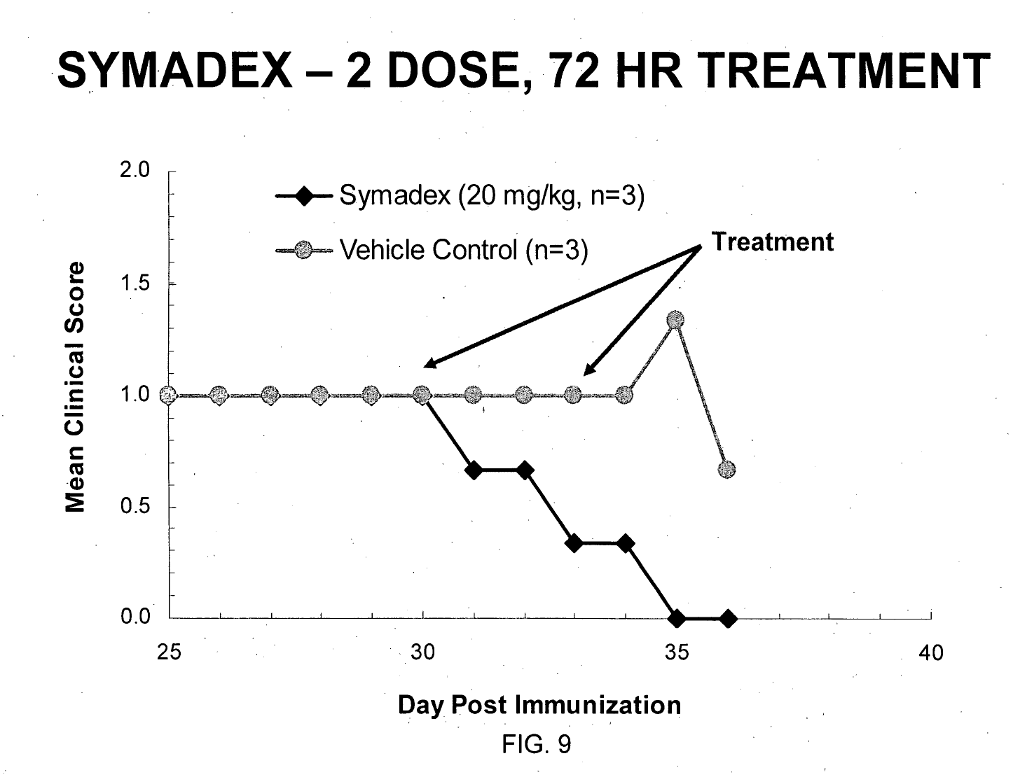

As shown in FIG. 9, the Symadex™ treatment on a 72 hour schedule reversed the progression of disease and restored baseline clinical scores with just 2 doses, while disease progression continued in the control cohort. The difference is statistically significant by a p value of 0.002 by the rank-sum test.

This experiment demonstrates that a more frequent dosing of Symadex™ can be tailored to match the particular balance between drug residence time, the temporal properties of the assault by inflammatory cells on myelin, and the intrinsic processes of permissive remyelination. It would be reasonable, therefore, to expect that combinations of dosing regimens can be applied first to accelerate recovery from disease, by more frequent or intense schedules of drug delivery, and then to maintain the beneficial effects of inflammatory cell blockade with less frequent, but, periodic, booster doses. The “saw-tooth” patterns of treatment and disease reversion evidenced in FIG. 7, in Example 3, suggested that the effect of Symadex™ can be attenuated over a 2-3 day interval between doses. This experiment confirms that the efficacy of Symadex™ can be re-enforced through more frequent dose administration.

### Example 5

### Symadex™ Alleviates the Symptoms of Experimental Autoimmune Encephalomyelitis (EAE), an Animal Model of Acute Multiple Sclerosis Upon Daily Dosing Over the Initial Course of Disease Induction

As described in Example 2, the EAE model in the guinea pig is biphasic. After the myelin basic protein insult on initial immunization, the typical clinical pattern of neurological impairment begins with acute signs of disease day 9 post immunization. Clinical onset results in weight loss, hind limb weakness and an abnormal righting reflex. The severity of these symptoms peaks over 6-7 additional days followed by a short duration transient and partial resolution by day until day-20, when the disease course changes to a steady progressive decline, from which there is no clinical recovery.

As an important extension to the utility of Symadex, its therapeutic effect at this earlier stage of disease presentation was examined. The experiment was further designed to build on the results of Example 4, which suggested that more frequent dosing affords more rapid and unidirectional symptom resolution. Since the acute phase of EAE also mimics active, but not necessarily progressive disease, as would be expected to be the case in human subjects with remitting-relapsing multiple sclerosis, the experiment was further designed to test the efficacy in comparison to mitoxantrone. This later drug, as indicated earlier, is an approved therapeutic agent and had served as the starting point for the chemical evolution of what became the Symadex™ molecule minus the toxicophores known to be causative agents for cardiotoxicity.

Accordingly, three randomized cohorts of guinea pigs with EAE induced by the method of Example 2, were treated, respectively, with 6 mg/kg of Symadex™ (full salt hydrate) and 0.35 mg/kg of mitoxantrone. Animals were treated daily, by intraperitoneal injection, for 15 days, starting on day 7 post immunization. Controls were treated with vehicle. We reasoned that 15 consecutive, 6 mg/kg doses of Symadex™ would represent a level of drug exposure that would be commensurate with the “20 mg/kg every 72 hours” regimen in Example 4 and consistent with a mid level exposure between the 20 mg/kg and the 40 mg/kg schedule given weekly, in Example 2. The mitoxantrone dose was selected to reflect a typical high dose given to rats or mice by daily dosing in the prior art, but allometrically scaled to the guinea pig.

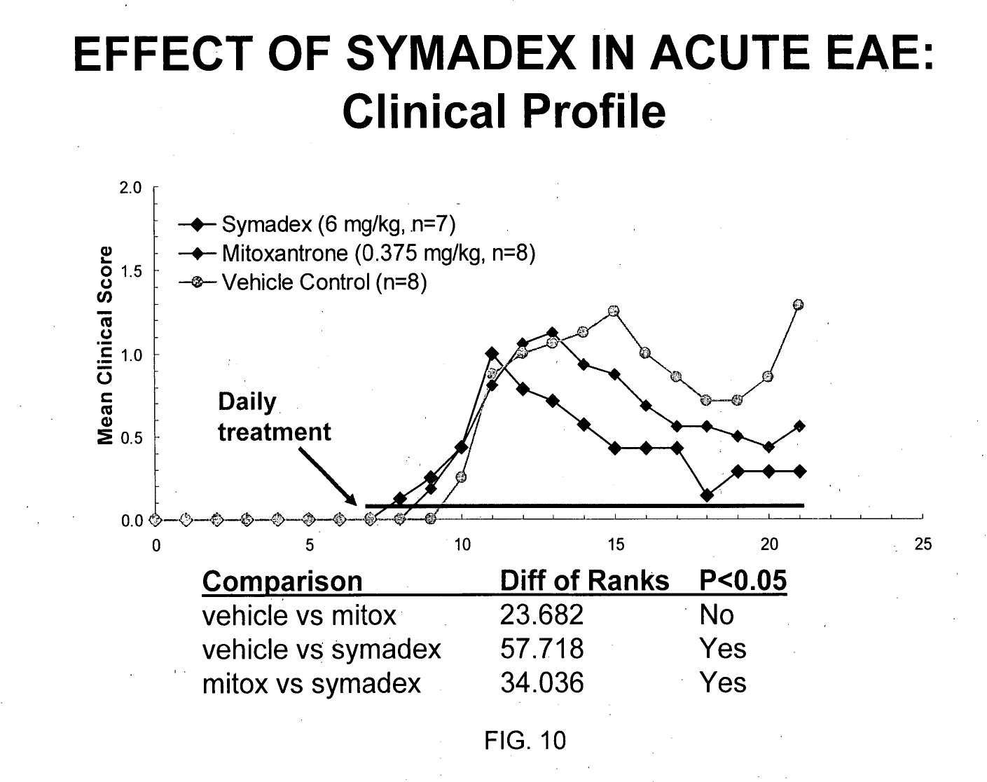

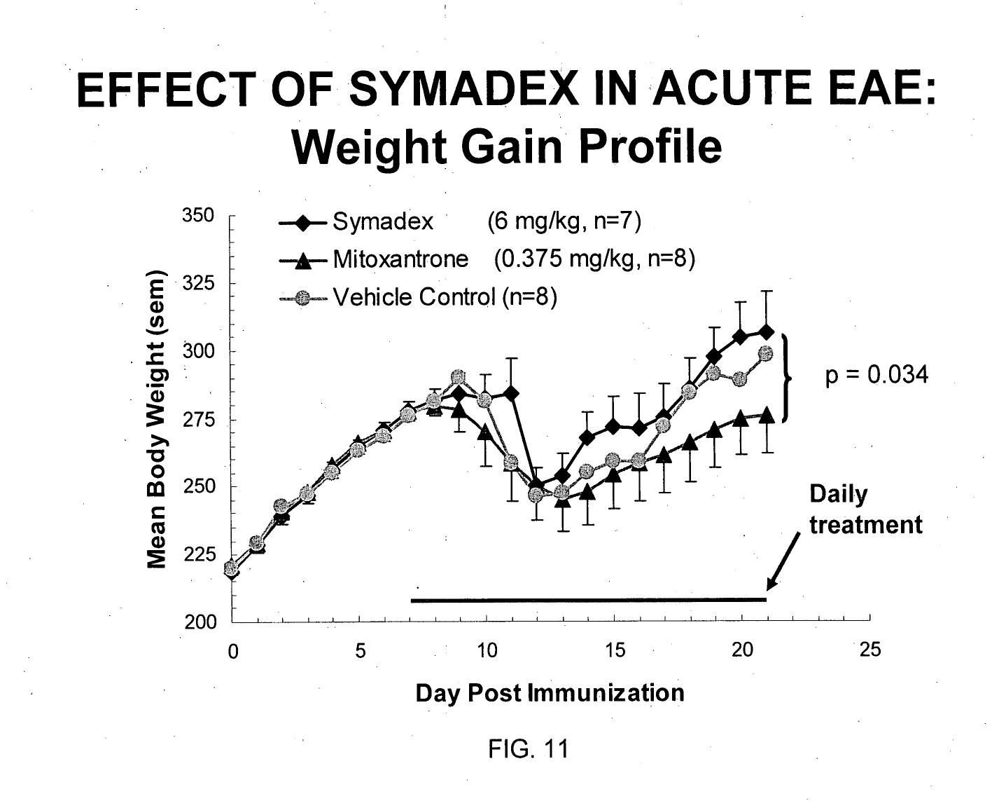

As can be appreciated from the results presented in FIG. 10, the cohorts treated with either Symadex™ or mitoxantrone present a consistently different trend than the vehicle controls. In the controls, the onset of disease is followed by a steady increase in clinical score until day 15, followed by the characteristic short term reversion and a second climb towards higher disease severity by day 20. In the case of both mitoxantrone and Symadex, the initial rise in neurological impairment is arrested, and all animals continued on a course of recovery towards basal levels throughout the dosing period. However, statistical analysis by Mann-Whitney rank-sum tests indicates that the therapeutic effect of Symadex™ against both control and the performance of mitoxantrone reaches statistical significance with a p value below 0.05. The difference in median scores between the performance of mitoxantrone and control did not reach statistical significance. FIG. 11 shows the weight gain profile, a sensitive indicator of general health status in guinea pigs. Acute EAE disease onset triggers a rapid weight loss from which there develops a steady recovery after day 15. Control animals and Symadex™ treated animals regain the ability to add weight every day, which is the norm for guinea pigs when healthy and prior to the onset of severe, chronic disease. However, under the circumstances of this experiment, mitoxantrone may have alleviated the acute clinical symptoms of EAE at a lower relative dose but it also impairs weight gain, a sign of generalized toxicological response to a drug that is a potent and broad spectrum cytotoxic. Comparison of the weight gain profiles between vehicle controls and Symadex™ did not show statistical significance by the Mann-Whitney rank-sum test, whereas the difference between Symadex™ and mitoxantrone reached statistical significance with a p value of 0.034.

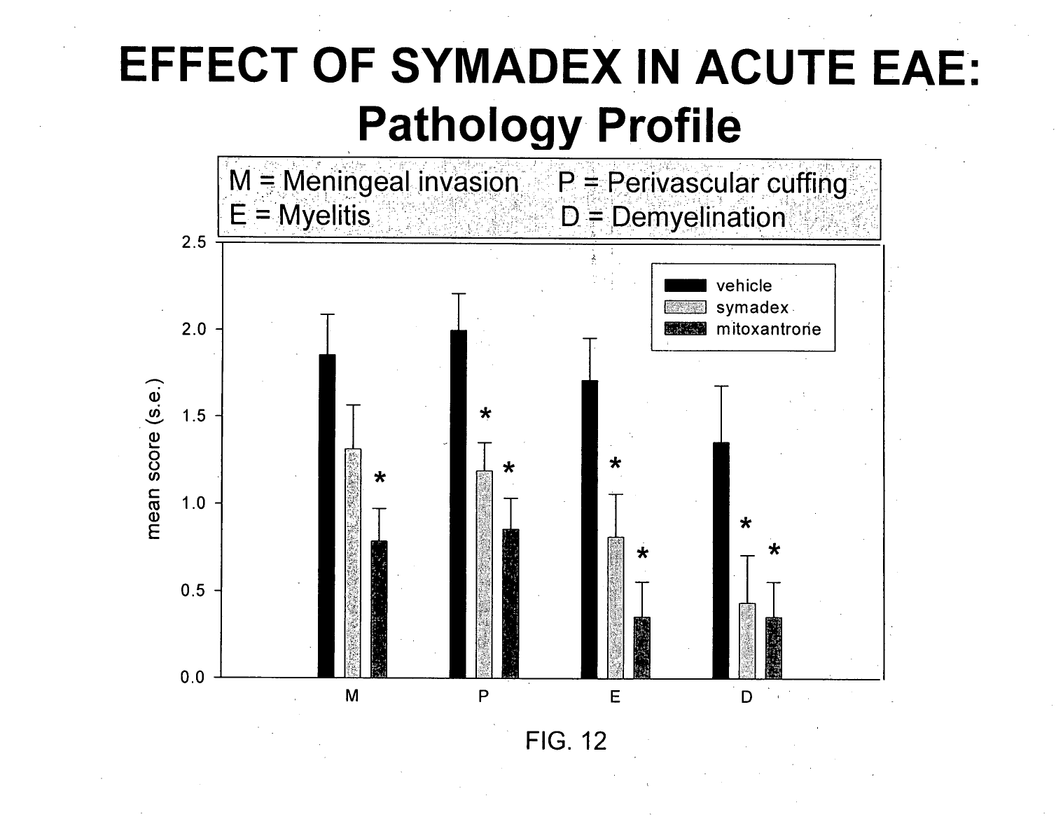

These results confirm that Symadex™ modifies the presentation of EAE throughout the course of active disease, both at the early acute and at the chronic phase without imposing a deleterious cytotoxic load. An analysis of the pathophysiology, shown in FIG. 12, confirms the earlier observations, by the histological methods described in Example 2, that Symadex™ arrests the invasion of parenchyma by inflammatory cells. It does so with a significant difference from the mode of action of mitoxantrone and drugs in the mitoxantrone class which are immunosuppressive. While the outcome may be the same in terms of the effect of both drugs on reducing the perivascular cuffing (P), myelitis (E) and demyelination (D), as judged by statistically significant differences (p value<0.05) from control, Symadex™ and mitoxantrone do not show the same action on meningeal inflammation (M). Mitoxantrone is an immunosuppressant that blocks activation and recruitment of inflammatory cells given the statistically significant reduction in meningeal inflammation (M). Symadex™ does not, although the therapeutic outcome according to the remaining three histopathological assessments is essentially similar.

These findings are relevant to the human disease circumstance, because it is considered highly beneficial to effect treatment of MS conditions without impairing the host's ability to mount an immunological, and hence inflammatory response, against adventitious infections. It terms of response to cytotoxic agents, which might impair gastrointestinal function and nutritional maintenance, the lack of negative effects on normal growth and weight gain in these guinea pig experiments points to another safety advantage that may accrue to Symadex™ therapy. The cumulative 15 day dose of Symadex™ for treatment of acute, active disease is 90 mg/kg. Allometric scaling to human dosimetry levels yields a corresponding human dose of 540 mg/m² body surface area (as free base), which has been shown to be a safe and well-tolerated single dose, and is lower than the 640 mg/m² dose which is indicated as a repeat dose every three weeks. Allometric scaling of the mitoxantrone dose, on the other hand, represents a total human equivalent dose of 45 mg/m². Since mitoxantrone is used in the treatment of MS on a three month dosing cycle at 12 mg/m², this represents the total dose for a year's worth of treatment. Thus, the experimental findings in this comparative example on the relative efficacy of Symadex™ versus mitoxantrone suggest that in humans a single dose of Symadex™ should show a similar, if not greater, therapeutic benefit as an year's course of mitoxantrone.

### Example 6

### Symadex™ Alleviates the Symptoms of Collagen Antibody Induced Arthritis in the Mouse, an Animal Model of Rheumatoid Arthritis and Autoimmune Disease

Rheumatoid Arthritis (RA) is an autoimmune disorder characterized by the chronic erosive inflammation in joints leading to the destruction of cartilage and bones. Several disease modifying antirheumatic drugs (DMARDS) are used in the treatment of RA. Currently, the two most important DMARDS are inhibitors of tumor necrosis factor α (TNF-α) and methotrexate (MTX). One method for demonstrating the utility of a pharmaceutical compound for the treatment of various conditions associated with RA is its ability to inhibit the induction of arthritis by collagen monoclonal antibodies (mABs) in mice.

Collagen-induced Arthritis (CIA) is an experimental autoimmune disease that can be elicited in susceptible strains of rodents (rat and mouse) and nonhuman primates by immunization with type II collagen, the major constituent protein of articular cartilage. CIA manifests as swelling and erythema in the limbs of the mouse. This model of autoimmunity shares several clinical and pathological features with rheumatoid arthritis (RA) and has become the most widely studied model of RA. CIA in the mouse model was first described by Courtenay et al. in 1980 (Courtnay, J. S., Dallman, M. J., Dayman, A. D., Martin A., and Mosedale, B. (1980) Immunisation against heterologous type II collagen induces arthritis in mice. Nature 283, 666-668). Like RA, susceptibility to CIA is regulated by the class II molecules of the major histocompatibility complex (MHC), indicating the crucial role played by T cells.

Methods

Groups of 3 BALB/c strain mice, 6-7 weeks of age, were used for the induction of arthritis by monoclonal antibodies (mABs) raised against type II collagen, plus lipopolysaccharide (LPS). A combination of 4 different mABs (D8, F10, DI-2G and A2) totaling 4 mg/mouse was administered to the animal intravenously on day 0, followed by intravenous challenge with 25 mg/mouse of LPS 72 hours later (day 3). From day 3, test substance and vehicle were each administered orally once daily for 3 consecutive days. For each animal, volumes of both hind paws were measured using a plethysmometer with water cell (12 mm diameter) on Days 0, 5, 7, 10, 14 and 17. Percent inhibition of increase in volume induced by mABs+LPS was calculated by the following formula:
Inhibition(%):[1−(Tn−T0)/(Cn−C0)]×100%
Where:

C0 (Cn): volume of day 0 (day n) in vehicle control

T0 (Tn): volume of day 0 (day n) in test compound-treated group

Reduction of edema in the hind paws by 30% or more is considered significant.

Results

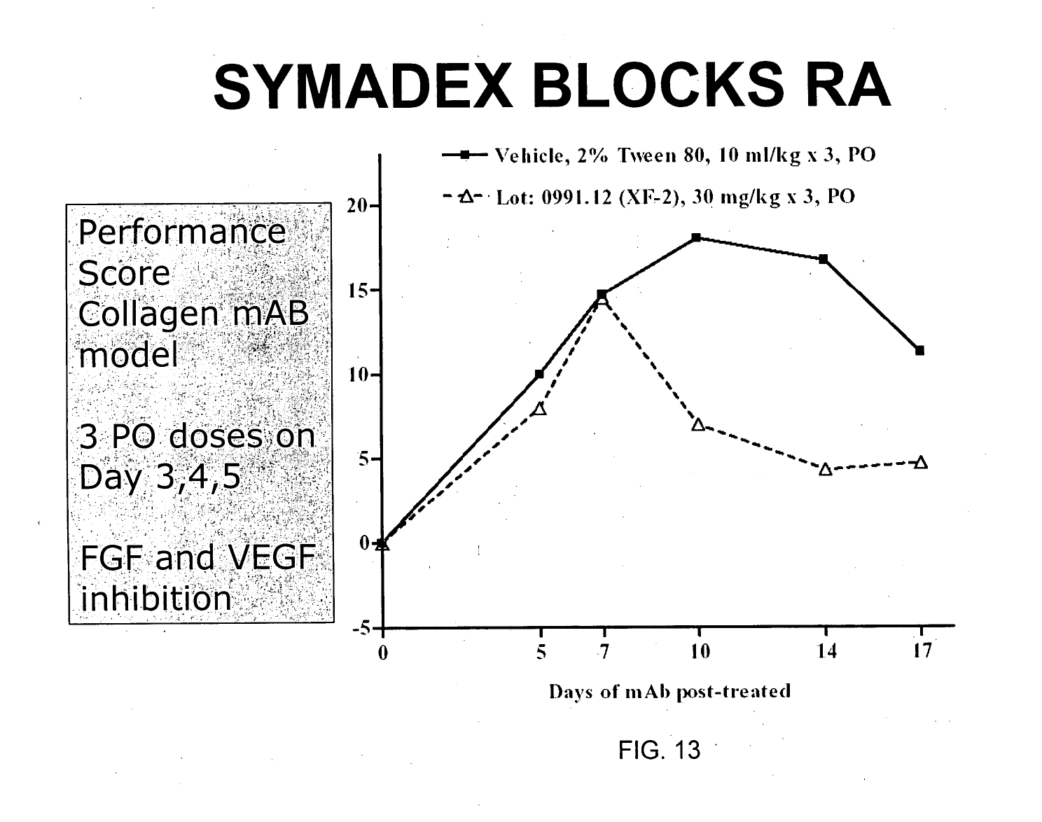

To monitor the onset of CIA, the volume of the two hind paws of mAB treated mice were measured. In the control (vehicle) treated animals the paws quickly became inflamed with a 42% increase in volume on day 5, the maximum volume was observed on day 10 and then the swelling began to subside. As shown in FIG. 13, in the Symadex™ treated group, the initial swelling on day 5 was slightly lower then control (32% vs. 42%) and significantly less inflammation (measured by paw volume) was observed on day 10 (29% vs. 75%), day 14 (18% vs. 70%) and day 17 (19% vs. 47%). The difference in means by paired t-test and in medians by non-parametric Mann-Whitney rank-sums all show p values lower than 0.01.

### CONCLUSION

Symadex™ demonstrated significant anti-arthritic activity in the mouse CIA model, with significant anti-inflammatory activity on day 10 (61% inhibition), day 14 (74% inhibition) and day 17 (59% inhibition). These findings are relevant in the context of prior example on EAE and autoimmune disease in general because they exemplify the efficacy of Symadex™ via an unexpected mechanism. The collagen antibody model of rheumatoid arthritis is significant because it by-passes the primary inflammatory insult of antigen presentation. Classical anti-inflammatories like the corticosteroids and anti-folates like methotrexate alleviate the consequence of autoimmune inflammatory diseases by suppressing the primary events of inflammatory cell activation and recruitment. The antibody induced model generates the symptoms of disease that present in the later stages of the autoimmune response, after activated cells become invasive into cartilage, having extravasated and transmigrated, as would be the case in MS during a prolonged assault on parenchyma.

Methotrexate, a benchmark therapeutic agent, has been shown to yield diminishing benefit in antibody induced models, which are intrinsically less dependent on T-cell activation than on their trafficking and migratory properties. The work of Lange et al. can be cited in this context (Annals of Rheumatoid Disease 64:599-605, 2005). By contrast, Symadex™ appears fully active in this model. The results presented in this example are especially relevant to the treatment of human subjects, because the therapeutic effect was obtained by oral administration. In the era of injectable biologics, such as blocking antibodies, the addition of an effective, non-immunosuppressive therapy via the oral route is particularly desirable.

### Example 7

### Symadex™ Downreaulates Otherwise Overexpressed Target Mechanisms of Inflammatory Cell Adhesion, Cell-Surface Signaling and Cell Proliferation

To explore the effect of Symadex™ treatment on gene expression, microarray experiments were performed.

Two colorectal cancer cell lines (HT29 & HCT116) were chosen for study, whose behavior as rapidly proliferating invasive cells could be generalized to many other such cell types from different tissue origins. The two lines were immortalized colon carcinomas. Their gene expression patterns are known to mimic the behavior of neuro-enteric cells and therefore provide an appropriate simulation of the kinds of regulatory patterns that would be found in cells of similar epithelial or endothelial origin. Cells with these ontological roots are also suitable models for the kinds of autoimmune and inflammatory susceptibilities that are common in tissues of neuroenteric origin, such as those in which inflammatory bowel disease would present itself.

Attention is drawn here to the exhaustive studies, using differential gene expression arrays (Zhang J. et al., “Neural system-enriched expression: relationship to biological pathways and neurological diseases”, Physiol. Genomics 18:167-183, 2004) which have documented the redundancies and commonalities of gene expression patterns in both the central nervous system and in anatomically unrelated tissues. For example, Zhang and colleagues, whose teachings are incorporated here by reference, profiled the expression products of 8,734 genes in 10 regions of the nervous system and in 30 peripheral organs. Their analyses reveal that approximately 70% of the genes relevant to nervous system diseases are also expressed in multiple tissues, including those of epithelial origin and in peripheral blood. These investigators suggest further that the profiling of genes implicated in nervous system diseases but sourced from various peripheral tissues, where easier sampling can be obtained, will aid the development of better mechanistic understanding about those diseases. Hence, the use of colon cells in gene expression studies as a model paradigm for understanding the effect of a drug on pathways common to those cells and nervous system tissues is experimentally justifiable.

Accordingly, the specific studies to document the mechanism of action of the compounds in the instant invention, were conducted as follows using the preferred imidazoacrinidone composition, referred to hereinafter as Symadex™.

Cells were grown in the presence of Symadex™ at the G150 concentration (0.68 and 0.21 ttMolar, for the HT29 and HCT116 cell lines, respectively), and harvested along with untreated control fractions after 1, 8 and 48 hrs. of exposure. Frozen cell pellets were lysed in triplicate and total RNA isolated by purification over spin columns (all reagents from Ambion). After QC acceptance for purity, total RNA was converted to cRNA by linear amplification and 10 μg samples were applied to CodeLink Human Whole Genome Bioarrays (GE Healthcare and GenUs Biosystems).

Arrays were processed in triplicate and comparisons made after robust statistical analysis of replicate variability. Genes (including ESTs) were considered to be differentially expressed if a change from baseline could be demonstrated as significant by T-test (p<0.05, α=0.025), using CodeLink Expression Analysis (GE Healthcare) and GeneSpring (Silicon Genetics) software. False discovery rates and representation in standardized gene ontologies/pathways were then determined by filtering the “fold” changes in expression with open access software packages, EASE and GoMiner, and with Pathways Analysis (Ingenuity Systems). Functional annotations were then explored further in the MedMiner literature search environment.

Over the interval sampled in the 24 hour test incubation, 271 down-regulated genes were significantly represented in both cell types, from within an array of 55,000 gene fragment accessions. A listing of these is shown in Table 3, in which the first column data presents the fold change against control, the second column cites the gene symbol, the third column cites the Genbank Accession, and the fourth column provides an abridged description of the gene's function.

TABLE 3
Significantly Downregulated Genes by the Action of Symadex ™

| MEAN | | | |
|---|---|---|---|
| FOLD CHANGE | | | |
| OVER | GENE | GENBANK | |
| CONTROL | SYMBOL | ACCESSION | DESCRIPTION |
| −17.00 | ACTA2 | AL713608 | actin, alpha 2, smooth muscle, aorta |
| −9.43 | ACVRL1 | NM_000020 | activin A receptor type II-like 1 |
| −2.07 | ACYP1 | AA664719 | acylphosphatase 1, erythrocyte (common) type |
| −21.74 | ADCYAP1 | NM_001117 | adenylate cyclase activating polypeptide 1 (pituitary) |
| −12.02 | ADH1C | NM_000669 | alcohol dehydrogenase 1C (class I), gamma polypeptide |
| −22.71 | AGT | NM_000029 | angiotensinogen (serine (or cysteine) proteinase inhibitor, |
| | | | clade A (alpha-1 antiproteinase, antitrypsin), member 8) |
| −2.71 | ALG5 | NM_013338 | asparagine-linked glycosylation 5 homolog (yeast, dolichyl- |
| | | | phosphate beta-glucosyltransferase) |
| −1.92 | ANAPC4 | NM_013367 | anaphase promoting complex subunit 4 |
| −13.15 | APOA1 | NM_000039 | apolipoprotein A-I |
| −2.29 | ARL6IP | NM_015161 | ADP-ribosylation factor-like 6 interacting protein |
| −2.21 | ASAH2 | AF250847 | N-acylsphingosine amidohydrolase (non-lysosomal |
| | | | ceramidase) 2 |
| −8.24 | ATP1B4 | AI659245 | ATPase, (Na+)/K+ transporting, beta 4 polypeptide |
| −9.43 | ATP2B3 | NM_021949 | ATPase, Ca++ transporting, plasma membrane 3 |
| −2.08 | BAD | NM_004322 | BCL2-antagonist of cell death |
| −2.22 | BAG2 | NM_004282 | BCL2-associated athanogene 2 |
| −23.65 | BBS2 | T26496 | Bardet-Biedl syndrome 2 |
| −2.17 | BCAP29 | NM_018844 | B-cell receptor-associated protein 29 |
| −12.57 | BGN | NM_001711 | biglycan |
| −1.91 | BIRC5 | NM_001168 | baculoviral IAP repeat-containing 5 (survivin) |
| −2.58 | BLM | NM_000057 | Bloom syndrome |
| −2.11 | C10ORF7 | NM_006023 | chromosome 10 open reading frame 7 |
| −15.52 | C3AR1 | NM_004054 | complement component 3a receptor 1 |
| −1.92 | CAMK1 | NM_003656 | calcium/calmodulin-dependent protein kinase I |
| −11.45 | CASR | BX106711 | calcium-sensing receptor (hypocalciuric hypercalcemia 1, |
| | | | severe neonatal hyperparathyroidism) |
| −12.99 | CCL23 | NM_005064 | chemokine (C—C motif) ligand 23 |
| −2.51 | CCNB2 | NM_004701 | cyclin B2 |
| −2.14 | CD164 | NM_006016 | CD164 antigen, sialomucin |
| −2.40 | CD58 | NM_001779 | CD58 antigen, (lymphocyte function-associated antigen 3) |
| −13.25 | CD5L | NM_005894 | CD5 antigen-like (scavenger receptor cysteine rich family) |
| −2.09 | CDC2 | NM_001786 | cell division cycle 2, G1 to S and G2 to M |
| −2.99 | CDC25C | NM_001790 | cell division cycle 25C |
| −8.14 | CDKL1 | NM_004196 | cyclin-dependent kinase-like 1 (CDC2-related kinase) |
| −19.89 | CENTA1 | NM_006869 | centaurin, alpha 1 |
| −2.14 | CHRNA5 | NM_000745 | cholinergic receptor, nicotinic, alpha polypeptide 5 |
| −2.38 | CKS1B | NM_001826 | CDC28 protein kinase regulatory subunit 1B |
| −54.48 | COL1A2 | NM_000089 | collagen, type I, alpha 2 |
| −2.54 | COPS3 | NM_003653 | COP9 constitutive photomorphogenic homolog subunit 3 |
| | | | (Arabidopsis) |
| −23.19 | COX6A2 | NM_005205 | cytochrome c oxidase subunit VIa polypeptide 2 |
| −2.13 | CREM | NM_001881 | cAMP responsive element modulator |
| −2.53 | CSE1L | NM_001316 | CSE1 chromosome segregation 1-like (yeast) |
| −26.26 | CSRP3 | NM_003476 | cysteine and glycine-rich protein 3 (cardiac LIM protein) |
| −20.65 | CYP19A1 | NM_000103 | cytochrome P450, family 19, subfamily A, polypeptide 1 |
| −1.88 | D8S2298E | NM_005671 | reproduction 8 |
| −1.94 | DCK | NM_000788 | deoxycytidine kinase |
| −3.14 | DCLRE1A | NM_014881 | DNA cross-link repair 1A (PSO2 homolog, S. cerevisiae) |
| −2.09 | DDX1 | NM_004939 | DEAD (Asp-Glu-Ala-Asp) box polypeptide 1 |
| −2.36 | DEK | NM_003472 | DEK oncogene (DNA binding) |
| −2.95 | DHFR | AU127142 | dihydrofolate reductase |
| −3.39 | DLEU2 | NM_006021 | deleted in lymphocytic leukemia, 2 |
| −2.15 | DNAJB11 | NM_016306 | DnaJ (Hsp40) homolog, subfamily B, member 11 |
| −2.24 | DNAJD1 | NM_013238 | DnaJ (Hsp40) homolog, subfamily D, member 1 |
| −89.97 | DSC3 | NM_001941 | desmocollin 3 |
| −9.75 | DSCR1L1 | NM_005822 | Down syndrome critical region gene 1-like 1 |
| −3.07 | DTYMK | NM_012145 | deoxythymidylate kinase (thymidylate kinase) |
| −2.18 | DUSP12 | NM_007240 | dual specificity phosphatase 12 |
| −2.08 | DUT | NM_001948 | dUTP pyrophosphatase |
| −28.71 | EGFL6 | NM_015507 | EGF-like-domain, multiple 6 |
| −15.24 | EIF2AK4 | AI630242 | eukaryotic translation initiation factor 2 alpha kinase 4 |
| −2.13 | EIF2S1 | NM_004094 | eukaryotic translation initiation factor 2, subunit 1 alpha, |
| | | | 35 kDa |
| −15.36 | EIF4EL3 | BX111619 | eukaryotic translation initiation factor 4E-like 3 |
| −6.35 | ENG | BM665467 | endoglin (Osler-Rendu-Weber syndrome 1) |
| −3.39 | ERH | NM_004450 | enhancer of rudimentary homolog (Drosophila) |
| −3.32 | FAIM | NM_018147 | Fas apoptotic inhibitory molecule |
| −2.11 | FARS1 | NM_006567 | phenylalanine-tRNA synthetase 1 (mitochondrial) |
| −17.42 | FBLN1 | NM_001996 | fibulin 1 |
| −13.00 | FBN1 | NM_000138 | fibrillin 1 (Marfan syndrome) |
| −11.62 | FCAR | NM_002000 | Fc fragment of IgA, receptor for |
| −2.87 | FEN1 | NM_004111 | flap structure-specific endonuclease 1 |
| −9.02 | FNTA | BI715309 | farnesyltransferase, CAAX box, alpha |
| −9.60 | FOXN1 | NM_003593 | forkhead box N1 |
| −10.14 | GABRA3 | NM_000808 | gamma-aminobutyric acid (GABA) A receptor, alpha 3 |
| −2.44 | GDAP1 | NM_018972 | ganglioside-induced differentiation-associated protein 1 |
| −1.96 | GGH | NM_003878 | gamma-glutamyl hydrolase (conjugase, |
| | | | folylpolygammaglutamyl hydrolase) |
| −19.73 | GIPR | NM_000164 | gastric inhibitory polypeptide receptor |
| −62.09 | GJB5 | NM_005268 | gap junction protein, beta 5 (connexin 31.1) |
| −2.51 | GLA | NM_000169 | galactosidase, alpha |
| −2.81 | GMNN | NM_015895 | geminin, DNA replication inhibitor |
| −18.16 | GNAL | BX116836 | guanine nucleotide binding protein (G protein), alpha |
| | | | activating activity polypeptide, olfactory type |
| −11.49 | GPR1 | CB992712 | G protein-coupled receptor 1 |
| −80.43 | GPR15 | NM_005290 | G protein-coupled receptor 15 |
| −13.80 | GPR24 | NM_005297 | G protein-coupled receptor 24 |
| −2.56 | GPR54 | NM_032551 | G protein-coupled receptor 54 |
| −2.31 | H2AFX | NM_002105 | H2A histone family, member X |
| −2.35 | H2AFZ | NM_002106 | H2A histone family, member Z |
| −2.63 | HAT1 | NM_003642 | histone acetyltransferase 1 |
| −1.97 | HMGB1 | NM_002128 | high-mobility group box 1 |
| −2.94 | HMMR | NM_012484 | hyaluronan-mediated motility receptor (RHAMM) |
| −7.42 | HNF4A | NM_000457 | hepatocyte nuclear factor 4, alpha |
| −1.93 | HNRPA2B1 | NM_002137 | heterogeneous nuclear ribonucleoprotein A2/B1 |
| −2.08 | HSGT1 | NM_007265 | suppressor of S. cerevisiae gcr2 |
| −13.76 | HSPB2 | NM_001541 | heat shock 27 kDa protein 2 |
| −58.56 | IBSP | NM_004967 | integrin-binding sialoprotein (bone sialoprotein, bone |
| | | | sialoprotein II) |
| −9.79 | IL13RA2 | NM_000640 | interleukin 13 receptor, alpha 2 |
| −11.23 | IL1RAP | AK095107 | interleukin 1 receptor accessory protein |
| −28.60 | IL1RL1 | NM_003856 | interleukin 1 receptor-like 1 |
| −25.20 | IL7R | NM_002185 | interleukin 7 receptor |
| −10.64 | ITGA2B | NM_000419 | integrin, alpha 2b (platelet glycoprotein IIb of IIb/IIIa |
| | | | complex, antigen CD41B) |
| −27.91 | ITGA9 | BF959890 | integrin, alpha 9 |
| −2.12 | ITGAE | NM_002208 | integrin, alpha E (antigen CD103, human mucosal |
| | | | lymphocyte antigen 1; alpha polypeptide) |
| −2.73 | ITGB3BP | NM_014288 | integrin beta 3 binding protein (beta3-endonexin) |
| −15.06 | ITSN1 | NM_003024 | intersectin 1 (SH3 domain protein) |
| −15.93 | KCNJ12 | NM_021012 | potassium inwardly-rectifying channel, subfamily J, |
| | | | member 12 |
| −6.83 | KCNJ15 | NM_002243 | potassium inwardly-rectifying channel, subfamily J, |
| | | | member 15 |
| −24.13 | KCNQ2 | NM_004518 | potassium voltage-gated channel, KQT-like subfamily, |
| | | | member 2 |
| −7.23 | KIAA0089 | NM_015141 | KIAA0089 protein |
| −2.52 | KIF2C | NM_006845 | kinesin family member 2C |
| −11.04 | KIF5A | AL118561 | kinesin family member 5A |
| −24.24 | KLRG1 | NM_005810 | killer cell lectin-like receptor subfamily G, member 1 |
| −3.34 | KRT13 | NM_002274 | keratin 13 |
| −11.85 | LCP2 | NM_005565 | lymphocyte cytosolic protein 2 (SH2 domain containing |
| | | | leukocyte protein of 76 kDa) |
| −57.61 | LIMS2 | NM_017980 | LIM and senescent cell antigen-like domains 2 |
| −87.48 | LNX | AL565198 | ligand of numb-protein X |
| −11.94 | LPAAT-E | NM_018361 | acid acyltransferase-epsilon |
| −2.40 | LPAAT-E | NM_018361 | acid acyltransferase-epsilon |
| −15.01 | LRP1B | NM_018557 | low density lipoprotein-related protein 1B (deleted in |
| | | | tumors) |
| −12.57 | LTB | NM_002341 | lymphotoxin beta (TNF superfamily, member 3) |
| −3.14 | MAD2L1 | NM_002358 | MAD2 mitotic arrest deficient-like 1 (yeast) |
| −12.89 | MAP6 | AB058781 | microtubule-associated protein 6 |
| −2.14 | MAPK13 | NM_002754 | mitogen-activated protein kinase 13 |
| −26.53 | MAPT | BM714794 | microtubule-associated protein tau |
| −1.89 | MAZ | NM_002383 | MYC-associated zinc finger protein (purine-binding |
| | | | transcription factor) |
| −1.97 | MCM6 | NM_005915 | MCM6 minichromosome maintenance deficient 6 (MIS5 |
| | | | homolog, S. pombe) (S. cerevisiae) |
| −2.69 | MCM7 | NM_005916 | MCM7 minichromosome maintenance deficient 7 (S. cerevisiae) |
| −2.39 | MEA | NM_014623 | male-enhanced antigen |
| −10.83 | MGAT4A | AI364966 | mannosyl (alpha-1,3-)-glycoprotein beta-1,4-N- |
| | | | acetylglucosaminyltransferase, isoenzyme A |
| −2.20 | MIS12 | NM_024039 | homolog of yeast Mis12 |
| −2.04 | MPZL1 | NM_003953 | myelin protein zero-like 1 |
| −2.02 | MRPL1 | NM_020236 | mitochondrial ribosomal protein L1 |
| −2.97 | MRPL11 | NM_016050 | mitochondrial ribosomal protein L11 |
| −2.16 | MRPL13 | NM_014078 | mitochondrial ribosomal protein L13 |
| −2.29 | MRPL23 | NM_021134 | mitochondrial ribosomal protein L23 |
| −2.20 | MRPL39 | NM_017446 | mitochondrial ribosomal protein L39 |
| −17.46 | MSLN | NM_005823 | mesothelin |
| −16.21 | MT1A | BM684446 | metallothionein 1A (functional) |
| −2.09 | MT2A | BG505162 | metallothionein 2A |
| −59.05 | MTIF2 | AI064964 | I factor (complement) |
| −2.66 | MXD3 | BQ053282 | MAX dimerization protein 3 |
| −21.22 | MYBPC2 | NM_004533 | myosin binding protein C, fast type |
| −50.59 | MYO15A | NM_016239 | myosin XVA |
| −33.67 | NCF1 | BI021745 | neutrophil cytosolic factor 1 (47 kDa, chronic |
| | | | granulomatous disease, autosomal 1) |
| −21.83 | NCF2 | NM_000433 | neutrophil cytosolic factor 2 (65 kDa, chronic |
| | | | granulomatous disease, autosomal 2) |
| −2.51 | NDUFA6 | NM_002490 | NADH dehydrogenase (ubiquinone) 1 alpha subcomplex, |
| | | | 6, 14 kDa |
| −15.58 | NDUFB3 | NM_002491 | NADH dehydrogenase (ubiquinone) 1 beta subcomplex, 3, |
| | | | 12 kDa |
| −10.52 | NDUFV3 | AW139027 | NADH dehydrogenase (ubiquinone) flavoprotein 3, 10 kDa |
| −6.89 | NEB | AI079911 | nebulin |
| −16.69 | NFATC1 | NM_006162 | nuclear factor of activated T-cells, cytoplasmic, |
| | | | calcineurin-dependent 1 |
| −2.01 | NFKBIB | NM_002503 | nuclear factor of kappa light polypeptide gene enhancer in |
| | | | B-cells inhibitor, beta |
| −2.41 | NMI | NM_004688 | N-myc (and STAT) interactor |
| −2.12 | NMU | NM_006681 | neuromedin U |
| −15.94 | NR0B1 | NM_000475 | nuclear receptor subfamily 0, group B, member 1 |
| −54.82 | NR2E1 | NM_003269 | nuclear receptor subfamily 2, group E, member 1 |
| −14.89 | NR2E3 | NM_016346 | nuclear receptor subfamily 2, group E, member 3 |
| −17.54 | NRG1 | NM_013956 | neuregulin 1 |
| −2.08 | NT5C3 | AA188573 | 5′-nucleotidase, cytosolic III |
| −17.10 | NT5E | BM994339 | 5′-nucleotidase, ecto (CD73) |
| −2.34 | NTHL1 | NM_002528 | nth endonuclease III-like 1 (E. coli) |
| −2.02 | NUCKS | NM_022731 | nuclear ubiquitous casein kinase and cyclin-dependent |
| | | | kinase substrate |
| −1.89 | NUDT1 | NM_002452 | nudix (nucleoside diphosphate linked moiety X)-type motif 1 |
| −2.68 | NUP107 | NM_020401 | nucleoporin 107 kDa |
| −16.33 | OLR1 | CD678960 | lysyl oxidase |
| −2.68 | OXCT | NM_000436 | 3-oxoacid CoA transferase 1 |
| −12.85 | PAFAH1B1 | AI674778 | platelet-activating factor acetylhydrolase, isoform lb, alpha |
| | | | subunit 45 kDa |
| −2.25 | PAFAH1B2 | NM_002572 | platelet-activating factor acetylhydrolase, isoform lb, beta |
| | | | subunit 30 kDa |
| −2.03 | PAICS | NM_006452 | phosphoribosylaminoimidazole carboxylase, |
| | | | phosphoribosylaminoimidazole succinocarboxamide |
| | | | synthetase |
| −19.65 | PCDH7 | NM_032457 | BH-protocadherin (brain-heart) |
| −16.89 | PCSK2 | NM_002594 | proprotein convertase subtilisin/kexin type 2 |
| −2.21 | PDCD5 | NM_004708 | programmed cell death 5 |
| −16.85 | PDE11A | NM_016953 | phosphodiesterase 11A |
| −17.53 | PECAM1 | BG739826 | platelet/endothelial cell adhesion molecule (CD31 antigen) |
| −10.55 | PFKL | AK098228 | phosphofructokinase, liver |
| −2.17 | PHAX | NM_032177 | likely ortholog of mouse phosphorylated adaptor for RNA |
| | | | export |
| −2.95 | PHF5A | NM_032758 | PHD finger protein 5A |
| −3.73 | PIR51 | NM_006479 | RAD51-interacting protein |
| −2.39 | PLK4 | NM_014264 | polo-like kinase 4 (Drosophila) |
| −8.04 | PLXNA3 | BF926082 | plexin A3 |
| −15.36 | PMPCB | AK090763 | peptidase (mitochondrial processing) beta |
| −1.88 | POLA | NM_016937 | polymerase (DNA directed), alpha |
| −2.62 | POLE2 | NM_002692 | polymerase (DNA directed), epsilon 2 (p59 subunit) |
| −2.04 | POLE4 | NM_019896 | polymerase (DNA-directed), epsilon 4 (p12 subunit) |
| −2.24 | POLR3K | NM_016310 | polymerase (RNA) III (DNA directed) polypeptide K, 12.3 kDa |
| −2.49 | PPIH | NM_006347 | peptidyl prolyl isomerase H (cyclophilin H) |
| −26.54 | PPP1R9A | AB033048 | protein phosphatase 1, regulatory (inhibitor) subunit 9A |
| −36.93 | PPP2R5A | AA496141 | protein phosphatase 2, regulatory subunit B (B56), alpha |
| | | | isoform |
| −3.78 | PRDM1 | NM_001198 | PR domain containing 1, with ZNF domain |
| −3.47 | PRIM1 | NM_000946 | primase, polypeptide 1, 49 kDa |
| −25.92 | PRLR | AA708864 | prolactin receptor |
| −22.21 | PRSS21 | NM_006799 | protease, serine, 21 (testisin) |
| −18.91 | PTPRG | BC047734 | protein tyrosine phosphatase, receptor type, G |
| −2.43 | PTTG1 | NM_004219 | pituitary tumor-transforming 1 |
| −6.00 | PXN | AW969600 | paxillin |
| −2.31 | RACGAP1 | NM_013277 | Rac GTPase activating protein 1 |
| −2.28 | RAD18 | NM_020165 | RAD18 homolog (S. cerevisiae) |
| −2.21 | RAD51 | NM_002875 | RAD51 homolog (RecA homolog, E. coli) (S. cerevisiae) |
| −2.04 | RAD54B | NM_012415 | RAD54 homolog B (S. cerevisiae) |
| −84.42 | RB1 | BI769614 | retinoblastoma 1 (including osteosarcoma) |
| −8.81 | RBBP9 | NM_006606 | retinoblastoma binding protein 9 |
| −7.09 | RCOR1 | NM_015156 | REST corepressor 1 |
| −2.02 | RFC4 | NM_002916 | replication factor C (activator 1) 4, 37 kDa |
| −2.31 | RNASEH2A | NM_006397 | ribonuclease H2, large subunit |
| −2.29 | RNF141 | NM_016422 | ring finger protein 141 |
| −11.95 | ROBO4 | NM_019055 | roundabout homolog 4, magic roundabout (Drosophila) |
| −2.54 | RPA3 | NM_002947 | replication protein A3, 14 kDa |
| −2.22 | RPC62 | NM_006468 | polymerase (RNA) III (DNA directed) polypeptide C (62 kD) |
| −39.17 | RPL4 | BF308998 | ribosomal protein L4 |
| −68.37 | RPS3 | BM693455 | ribosomal protein S3 |
| −2.38 | RQCD1 | NM_005444 | RCD1 required for cell differentiation1 homolog (S. pombe) |
| −2.43 | RYR3 | BU533957 | ryanodine receptor 3 |
| −2.14 | SARA1 | NM_020150 | SAR1a gene homolog 1 (S. cerevisiae) |
| −2.00 | SCAMP3 | NM_005698 | secretory carrier membrane protein 3 |
| −14.52 | SCNN1G | NM_001039 | sodium channel, nonvoltage-gated 1, gamma |
| −16.54 | SEMA7A | NM_003612 | sema domain, immunoglobulin domain (Ig), and GPI |
| | | | membrane anchor, (semaphorin) 7A |
| −2.84 | SFRS3 | NM_003017 | splicing factor, arginine/serine-rich 3 |
| −7.34 | SHOX | NM_000451 | short stature homeobox |
| −11.76 | SIAT7A | NM_018414 | sialyltransferase 7 ((alpha-N-acetylneuraminyl-2,3-beta- |
| | | | galactosyl-1,3)-N-acetyl galactosaminide alpha-2,6- |
| | | | sialyltransferase) A |
| −2.93 | SIN3B | AW051366 | SIN3 homolog B, transcriptional regulator (yeast) |
| −2.78 | SIVA | NM_006427 | CD27-binding (Siva) protein |
| −11.21 | SLC1A2 | NM_004171 | solute carrier family 1 (glial high affinity glutamate |
| | | | transporter), member 2 |
| −11.51 | SLC22A13 | NM_004256 | solute carrier family 22 (organic cation transporter), |
| | | | member 13 |
| −18.72 | SLC27A6 | NM_014031 | solute carrier family 27 (fatty acid transporter), member 6 |
| −2.23 | SLC35B1 | NM_005827 | solute carrier family 35, member B1 |
| −19.44 | SLC6A4 | NM_001045 | solute carrier family 6 (neurotransmitter transporter, |
| | | | serotonin), member 4 |
| −27.75 | SLC7A13 | NM_138817 | solute carrier family 7, (cationic amino acid transporter, y+ |
| | | | system) member 13 |
| −15.57 | SLC9A3 | NM_004174 | solute carrier family 9 (sodium/hydrogen exchanger), |
| | | | isoform 3 |
| −8.20 | SLC9A7 | AA279477 | solute carrier family 9 (sodium/hydrogen exchanger), |
| | | | isoform 7 |
| −17.45 | SNAI2 | NM_003068 | snail homolog 2 (Drosophila) |
| −2.31 | SNRPD3 | NM_004175 | small nuclear ribonucleoprotein D3 polypeptide 18 kDa |
| −14.75 | SPO11 | NM_012444 | SPO11 meiotic protein covalently bound to DSB-like (S. cerevisiae) |
| −6.57 | SPOCK | NM_004598 | sparc/osteonectin, cwcv and kazal-like domains |
| | | | proteoglycan (testican) |
| −16.71 | SPTB | NM_000347 | spectrin, beta, erythrocytic (includes spherocytosis, clinical |
| | | | type I) |
| −13.79 | SRY | NM_003140 | sex determining region Y |
| −2.20 | SSSCA1 | NM_006396 | Sjogren's syndrome/scleroderma autoantigen 1 |
| −1.92 | STK6 | NM_003600 | serine/threonine kinase 6 |
| −2.41 | STMN1 | NM_005563 | stathmin 1/oncoprotein 18 |
| −10.91 | SULT1E1 | NM_005420 | sulfotransferase family 1E, estrogen-preferring, member 1 |
| −293.18 | SULT4A1 | NM_014351 | sulfotransferase family 4A, member 1 |
| −2.33 | SUV39H2 | NM_024670 | suppressor of variegation 3-9 homolog 2 (Drosophila) |
| −2.23 | SYNCRIP | NM_006372 | synaptotagmin binding, cytoplasmic RNA interacting |
| | | | protein |
| −1481.77 | SYNE1 | NM_033071 | spectrin repeat containing, nuclear envelope 1 |
| −12.26 | TAC3 | NM_013251 | tachykinin 3 (neuromedin K, neurokinin beta) |
| −2.04 | TADA2L | NM_001488 | transcriptional adaptor 2 (ADA2 homolog, yeast)-like |
| −9.82 | TCP11 | NM_018679 | t-complex 11 (mouse) |
| −11.00 | TFAP2A | NM_003220 | transcription factor AP-2 alpha (activating enhancer |
| | | | binding protein 2 alpha) |
| −20.30 | TFEC | NM_012252 | transcription factor EC |
| −3.41 | THOC4 | NM_005782 | THO complex 4 |
| −2.23 | TIMM10 | NM_012456 | translocase of inner mitochondrial membrane 10 homolog |
| | | | (yeast) |
| −2.02 | TIMM23 | NM_006327 | translocase of inner mitochondrial membrane 23 homolog |
| | | | (yeast) |
| −2.34 | TK1 | NM_003258 | thymidine kinase 1, soluble |
| −2.21 | TMEM4 | NM_014255 | transmembrane protein 4 |
| −2.64 | TMPO | H57815 | thymopoietin |
| −36.20 | TNP1 | NM_003284 | transition protein 1 (during histone to protamine |
| | | | replacement) |
| −13.71 | TPSD1 | NM_012217 | tryptase delta 1 |
| −2.55 | TRA2A | BF093914 | transformer-2 alpha |
| −8.14 | TRH | NM_007117 | thyrotropin-releasing hormone |
| −2.11 | TSFM | AW603708 | Ts translation elongation factor, mitochondrial |
| −2.24 | TTK | NM_003318 | TTK protein kinase |
| −2.13 | TXNDC | NM_030755 | thioredoxin domain containing |
| −12.47 | TYRP1 | NM_000550 | tyrosinase-related protein 1 |
| −1.99 | U2AF1 | NM_006758 | U2(RNU2) small nuclear RNA auxiliary factor 1 |
| −10.12 | UBL4 | AA873769 | ubiquitin-like 4 |
| −1.92 | UMPK | NM_012474 | uridine monophosphate kinase |
| −66.26 | USP16 | NM_006447 | ubiquitin specific protease 16 |
| −12.46 | VAPA | AI671488 | VAMP (vesicle-associated membrane protein)-associated |
| | | | protein A, 33 kDa |
| −2.22 | VDAC3 | NM_005662 | voltage-dependent anion channel 3 |
| −2.79 | VRK1 | NM_003384 | vaccinia related kinase 1 |
| −2.07 | WWOX | NM_016373 | WW domain containing oxidoreductase |
| −30.18 | ZNF145 | BU607554 | zinc finger protein 145 (Kruppel-like, expressed in |
| | | | promyelocytic leukemia) |
| −2.66 | ZNF258 | NM_007167 | zinc finger protein 258 |
| −2.03 | ZNF265 | NM_005455 | zinc finger protein 265 |
| −31.24 | ZNF282 | NM_003575 | zinc finger protein 282 |
| −2.17 | ZNRD1 | NM_014596 | zinc ribbon domain containing, 1 |
| −2.31 | ZW10 | NM_004724 | ZW10 homolog, centromere/kinetochore protein |
| | | | (Drosophila) |

Analysis

Review of this listing in the context of gene ontology, reveals that Symadex™ exerts a profound, if pleiotropic effect, on mechanisms of cell aggregation and proliferation and on processes associated with invasive cellular growth, which are the hallmark of the inflammatory etiology associated with the autoimmune diseases described at the outset.

More detailed analysis of the evidence in Table 3 reveals, for example, that a significant proportion of the down regulated genes are associated with mechanisms of cell surface signaling, motility, migration and adhesion, which permit inflammatory cells to cross vascular barrier and penetrate into parenchymal layers. Those practiced in the art will recognize that these ontological relationships are described more fully in literature within databases in the public domain, from which the following information has been excerpted. Those databases include DAVID (Database for Annotation, Visualization and Integrated Discovery, from the National Institute of Allergy and Infectious Disease, http://apps1.niaid.nih.gov/david/; the sister program EASE (Expression Analysis Systematic Explorer) at the same site; and the GeneCards bioinformatics project (http://genome-www.stanford.edu/genecards/index.shtml).

For example, in the differential gene expression experiment under discussion, the down regulated genes ACTA2, ACVRL1, BGN, DSC3, ENG, FBAN1, FBLN1, HMMR, IGTA2B, ITGA2B, ITGA9, ITGAE, LIMS2, LTB, MAPT, MSLN, NMI, PCDH7, PECAM 1, PRDM 1, SEMA7A, VAPA all participate in the regulation of these processes via direct modulation of adhesion factors, like integrins and cadherins, or by disrupting the growth factor signals that promote their expression and the assembly of accessory proteins that further facilitate the adhesion process. Of special importance in this context is the remarkable 1500 fold down-regulation of the SYNE1 spectrin repeats. The accessory proteins in the nesprin family coded by this gene maintain nuclear organization and the structural integrity of the cellular cytoskeleton, Down-regulation of SYNE1 would be expected to impair the ability of inflammatory cells to maintain their shape and geometry during periods of invasive motility. Thus, this effect of Symadex™ on differential gene expression of the machinery for maintaining cellular conformation would yield to the collapse of those cells during trafficking, an outcome also consistent with the histopathology of Symadex's therapeutic mode of action.

The requisite processes for calcium ion and high energy phosphate generation are affected in tandem as evidenced by the down regulation of ATP1B4, ATP2B3, CAMK1, EGFL6, GPR24, IBSP, NUDT1, RAD54B, RYR3, and SLC9A7. Cell proliferation in turn is put in check through cell cycle blocking processes mediated by B1RC5, CCL23, CCNB2, CDC2, CDC25C, CKS1B, CREM, EGFL6, FCAR, IL13RA2, IL1RAP, IL1RL1, MAPK13, NRG1, PTPRG, STK6 among other such related genes. Neuromodulation via paracrine and autocrine controls is also evident in the downregulation of systems that further respond to neuroinflammatory insult, including, for example, neurotransmitter transporters associated with damaging, runaway glutamate signaling. The downregulated genes in this latter category are exemplified by ADCYAP1, GABRA3, GGH, KCNQ3, SLC1A2 (and its SLC family solute carrier homologs), and SULT4A1. This latter gene showed close to 300 fold down-regulation. It is a gene associated with heparan sulfation. Sulfated heparans constitute the “molecular velcro” that permits integrins to bind to laminins and thereby provide the linkage that permits invasive inflammatory cells to transmigrate through basal membranes into CNS parenchyma. Down regulation of a such a process would be expected to keep inflammatory cells within the confines of vascular cuffs, as has been observed to be the case in the histopathological evaluation on the Symadex™ treatment effect noted in Examples 2-8.

The integrated function of these genes affected by Symadex™ is consistent with the differential expression profile that has been observed with microarray experiments, as for example, in the work of Arnett H A et al., “Functional genomic analysis of remyelination reveals importance of inflammation in oligodendrocyte regeneration”, J. Neuroscience 23(30):9824-9832, 2003; Lindberg RLP et al., “Multiple sclerosis as a generalized CNS disease-comparative microarray analysis of normal appearing white matter and lesions in secondary progressive MS”, J. Neuroimmunology 152:154-167, 2004; and Tajouri L. et al., “Quantitative and qualitative changes in gene expression patterns characterize the activity of plaques in multiple sclerosis”, Mol. Brain Res. 119:170-183, 2003. These studies have cataloged, in a similar manner to the gene descriptions presented here, the characteristics of representative autoimmune inflammatory insults and subsequent recovery therefrom, especially in the context of autoimmune demyelinating models for which multiple sclerosis serves as a prime circumstance. Therefore, the assertion that the application of Symadex™ and its congeners in therapy for multiple sclerosis, and autoimmune diseases of similar etiology, is demonstrable in terms of the compound's molecular pharmacology.

### Example 8

### Synthesis of the Indazoles of Formula (IA)

Compounds of formula (IA) can be synthesized according to the following sequence of steps, starting with 2-Fluoro-5-nitro-6-(1H-indazol-5-ylamino)benzoic acid.

### 1) 2-Fluoro-5-nitro-6-(1H-indazol-5-ylamino)benzoic acid

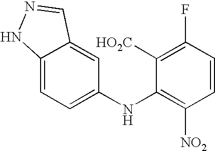

3-Nitro-2,6-difluorobenzoic acid (20.0 g, 98.44 mmol) was added to a solution of ethanol (100 mL) and water (100 mL). The acid solution was cooled to 10° C. and triethylamine (25.09 mL) added dropwise under rapid stirring to ensure the temperature did not exceed 40° C. 5-Aminoindazole (13.01 g, 98.44 mmol) was then added in portions and the combined mixture heated to 70° C. for 16 hours. A solution of water (100 mL) and conc. HCl (100 mL) was made, heated to 60° C. and placed under vigorous stirring. The reaction mixture, still at 70° C., was transferred to the HCl/water solution in small portions and allowed to cool to room temperature. The mixture was stirred for a further 4 hours to ensure maximum precipitation. The resulting precipitate was filtered off and washed with water (2×60 mL) and dried in a vacuum oven overnight (28.3 g, 89.56 mmol, 94%). ¹H δ (d6-DMSO): 6.97 (1H, dd, ArH, J=8.8, 2.4 Hz), 7.18 (1H, d, ArH, J=2.4 Hz), 7.43 (1H, d, ArH, J=8.8 Hz), 7.45 (1H, d, ArH, J=8.8 Hz), 8.10 (1H, d, ArH, J=8.8 Hz), 8.99 (1H, s, ArH), 9.15 (1H, s, NH), 9.87 (1H, s, NH); HPLC: Rₜ=3.52 min.; LRMS: m/z=315.4 (M−H)

### 2) 1-Fluoro-4-nitropyrazolo[7,8a]-acridin-9(10H)-one

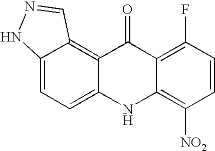

A suspension of 2-fluoro-5-nitro-6-(1H-indazol-5-ylamino)benzoic acid (20.0 g, 65.096 mmol) in chloroform under argon had freshly distilled POCl₃ (24.3 mL, 260.383 mmol) added to it. The mixture was heated to 80° C. for 16 hours. Once cooled, ethanol (50 mL) was added slowly to quench the excess POCl₃ reaction and then stirred for 30 minutes at room temperature. At this point, the mixture was reduced to dryness under vacuum. Water (100 mL) was added to the residue and saturated sodium bicarbonate solution added until the pH=8. The mixture was then stirred for 60 minutes at room temperature and the precipitate filtered off, washed with water (2×80 mL) and dried overnight in a vacuum oven (19.219 g, 64.445 mmol, 99%). ¹H δ (d6-DMSO): NMR unavailable since compound very insoluble; HPLC: Rₜ=6.22 min.; LRMS: m/z=299.1 (M+H)

### 3) Compound (VIA):1-Fluoro-4-nitropyrazolo[7,8a]-imidazo[4,5,1-de]-acridin-9(10H)-one

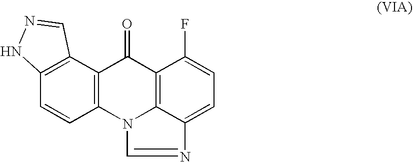

To a slurry of starting material (7.112 g, 23.846 mmol) in formic acid (75 mL), was added SnCl₂.2H₂O (23.07 g, 102.25 mmol) in conc. HCl (15 mL). The combined mixture was stirred at room temperature for 30 minutes then heated to 95° C. for 20 hours. Once cooled, the solid was filtered off and partially dried before being slurried in sat. sodium bicarbonate solution and stirred for 60 mins. The precipitate was then filtered off and washed with water (2×1100 mL) and dried in a vacuum oven overnight (6.303 g, 22.654 mmol, 95%).

### 4) Compound (VA)

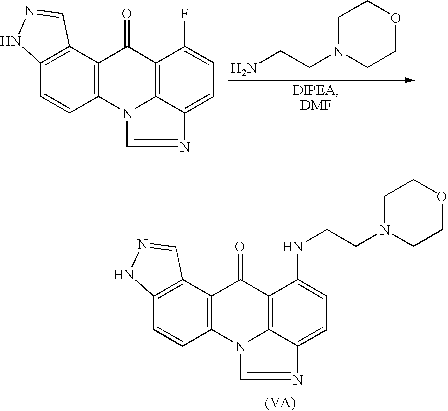

Amide core (0.294 g, 1.057 mmol), 2-morpholine ethylamine (0.220 g, 1.961 mmol, 0.222 mL), diisopropylethylamine (0.273 g, 2.114 mmol, 0.368 mL) and dimethylacetamide (2.0 mL) were combined in a 5 mL reaction tube and heated to 150° C. under microwave irradiation for 20 minutes. Once cooled, the solvent was removed and the residue columned over silica gel (0-20% MeOH in CHCl₃). The desired fractions were pooled and the solvent removed to yield the target compound. ¹H δ (d6-DMSO): 2.41 (2H, m, CH₂NCH₂), 2.68 (2H, m, CH₂), 3.19 (2H, m, CH₂NCH₂), 3.36 (2H, m, NHCH₂), 3.63 (4H, m, CH₂O CH₂), 6.89 (1H, d, ArH, J=8.6 Hz), 8.01 (1H, d, ArH, J=8.6 Hz), 8.16 (1H, d, ArH, J=9.0), 8.44 (1H, d, ArH, J=9.0 Hz), 9.00 (1H, s, Pyrazole-H), 9.07 (1H, m, NH), 9.29 (1H, s, Imidazole-H), 13.69 (1H, s, N═NH); HPLC: Rₜ=3.55 min.; LRMS: 389.0 (M+1).

Additional compounds with the fused pyrazole scaffold, for example, compounds (IIIA) and (IVA),

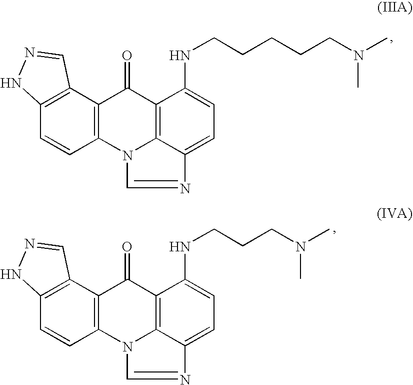

are prepared in a similar manner, following the method used for (VA), namely, by treating the amide core, compound (VIA), with other dialkylamino-alkyl amines and the same reaction stoichiometry used in the model reaction with 2-morpholine ethylamine. For example, compounds (IIIA) and (IVA) were prepared by the addition of 5-N,N-dimethylamino pentylamine and 3-N,N-dimethylamino propylamine to compound (VIA). The resulting compounds conformed to theory as evidenced by their NMR and mass spectroscopic features, shown in the accompanying table that provides a handy comparison of the analytical properties for the precursor amide core, compound (VIA), and it derivatives.

TABLE 4
Spectroscopic data on the pyrazole core and additional analogs prepared in
the same manner as XF-122

| | | LRMS | HPLC |
|---|---|---|---|
| Structure | ¹H NMR (δ) | (m/z) | (Rₜ) |
| 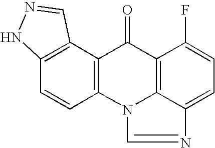 | 7.45 (1H, dd, ArH, J = 8.8, 8.8 Hz), 8.20 (1H, d, ArH, J = 9.2 Hz), 8.24 (1H, dd, ArH, J = 8.8, 3.4), 8.44 (1H, d, ArH, J = 9.2 Hz), 8.96 (1H, s, ArH), 9.64 (1H, s, ArH), 13.77 (1H, s, NH) | 279.0 (M + H) | 5.27 min. |
| 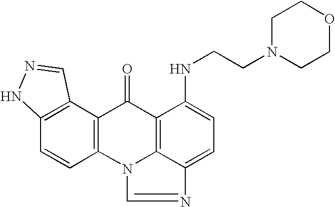 | 2.41 (2H, m, CH₂NCH₂), 2.68 (2H, m, CH₂), 3.19 (2H, m, CH₂NCH₂), 3.36 (2H, m, NHCH₂), 3.63 (4H, m, CH₂O CH₂), 6.89 (1H, d, ArH, J = 8.6 Hz), 8.01 (1H, d, ArH, J = 8.6 Hz), 8.16 (1H, d, ArH, J = 9.0 Hz), 8.44 (1H, d, ArH, J = 9.0 Hz), 9.00 (1H, s, ArH), 9.07 (1H, m, NH), 9.29 (1H, s, ArH), 13.69 | | |
| # (1H, s, N═NH) | 389.0 (M + H) | 3.52 min. | |
| 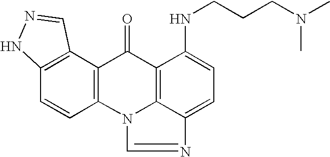 | 2.09 (2H, t, CH₂, J = 7.2 Hz), 2.75 (6H, s, N(CH₃)₂, 3.18 (2H, t, NCH₂, J = 7.2 Hz), 3.54 (2H, m, NHCH₂), 6.90 (1H, d, ArH, J = 9.2 Hz), 8.03 (1H, d, ArH, J = 9.2 Hz), 8.17 (1H, d, ArH, J = 9.2 Hz), 8.44 (1H, d, ArH, J = 19.2 Hz), 8.99 (1H, s, ArH), 9.03 (1H, m, NH), 9.30 (1H, s, ArH), 12.76 (1H, s, NH) | 361.0 | |
| # (M + H) | 3.56 min. | | |
| 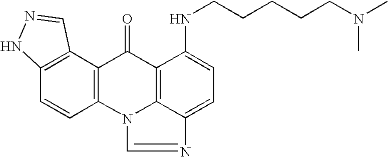 | 1.32 (2H, m, CH₂), 1.56 (2H, m, CH₂), 1.64 (2H, m, CH₂), 2.66 (6H, s, N(CH₃)₂), 2.74 (2H, m, CH₂), 2.93 (2H, CH₂), 6.86 (1H, d, ArH, J = 8.8 Hz), 8.01 (1H, d, ArH, J = 8.8 Hz), 8.16 (1H, d, ArH, J = 8.8 Hz), 8.45 (1H, d, ArH, J = 8.8 Hz), 8.99 (1H, s, ArH), 9.02 (1H, m, NH), 9.30 (1H, s, ArH) | 389.0 | |
| # (M + H) | 4.67 min. | | |

### Example 9

### Indazole Compounds of Formula (IA) Selectively Target FLT3 Protein Tyrosine Kinase In Vitro

The terms “IC50” and “EC50” are used interchangeably. As used herein, “EC50” refers to nMolar concentration at median percent inhibition determined by dose respose (DR) assay. As used herein, the term “E1000” refers to percent inhibition at 1000 nMolar determined by assay. EC50 was calculated based on dose response curve was fitted to 4 parameter Hill equation.

Testing of the compounds described in this invention was carried out using the SelectScreen™ platform from Invitrogen, Inc. (Carlsbad, Calif., USA) and the details of its performance are readily viewed via the web by linking to: http://www.invitrogen.com/downloads/SelectScrn_Brochure.pdf.

Briefly the approach is based on treating each specific kinase with a unique substrate and optical reporter system in the presence of ATP at 100 micromolar. In controls, the substrate is phosphorylated and a baseline optimal response is recorded.

Compounds were initially tested at 1000 nanomolar concentrations and the % inhibition of enzyme activity determined (E1000). The compounds were then re-tested by adding graded amounts of putative inhibitor which were added in 5 separate increments to generate a dose response curve. The latter is obtained by fitting to a 4 parameter Hill equation, a sigmoid saturation equation. The concentration which causes 50% enzyme inhibition (EC50) was then calculated from the dose response equation.

An effective level of inhibition in the low nanomolar range is considered to qualify the test compound as potential drug or targeting agent against the specific kinase that it has inhibited. The EC50 value is, therefore, a measure of potency. Another important feature is specificity. It is considered a desirable property when claiming efficacy to determine how many kinases are inhibited by the same molecule. The fewer number inhibited points toward specificity; the greater to inhibitory promiscuity.

For the compounds of this invention, the experimental condition were as follows. The 2×FLT3/Tyr 02 peptide mixture was prepared in 50 mM HEPES pH 7.5, 0.01 BRIJ-35, 10 mM MgCl2, 1 mM EGTA. The final 10 uL kinase reaction consists of 0.6-76.0 ng FLT3 and 2 uM Tyr 02 peptide in 50 mM HEPES pH 7.5, 0.01% BRIJ-35, mM Mg C12, 1 mM EGTA. After 1 hour kinase reaction incubation, 5 uL of a 1:64 dilution of development reagent A was added.

When tested in in vitro dose response assays against FLT3, compounds (1IIA), (IVA) and (VA) exhibited the following values of EC50 and E1000:

| Structure | EC50 | E1000 |
|---|---|---|
| 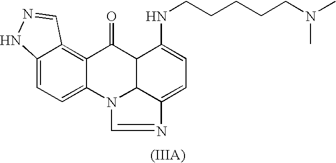 | 16 | 96 |
| 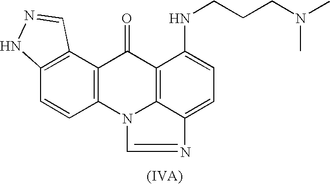 | 9 | 97 |
| 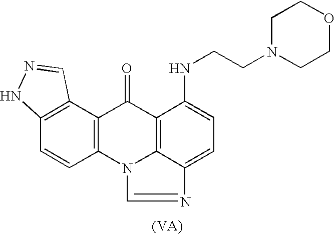 | 32 | 87 |

### Example 10

### Synthesis of the Compounds of Formula (IB)

Representative compounds of formula (I) can be synthesized according to the following synthetic schemes. The starting compound in each of the schemes below, referred to herein as C1311, can be synthesized according to a method disclosed in U.S. Pat. No. 6,229,015. The aforementioned patent is incorporated herein by reference.
Compound (VIB): Acetic acid 5-(2-diethylamino-ethylamino)-6-oxo-6H-2,10b-diaza-aceanthrylen-8-yl ester

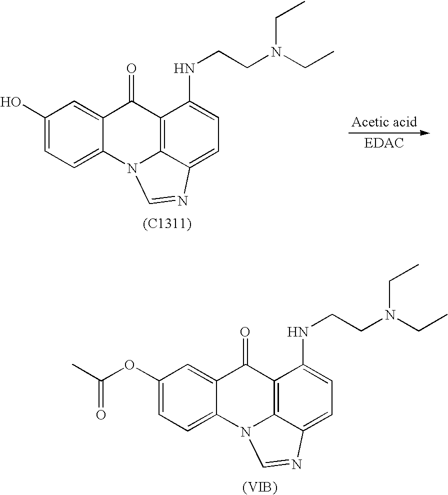

A solution of C1311 free base (2.11 g, 6.02 mmol), N-(3-dimethylaminopropyl)-N′-ethylcarbodiimide hydrochloride (1.73 g, 9.03 mmol), 4-(dimethylamino)pyridine (12 mg, 0.10 mmol), acetic acid (0.523 mL, 9.03 mmol), and N,N-diisopropylethylamine (2.35 mL, 13.5 mmol) in anhydrous DMF (36 mL) was stirred at room temperature for 20 hours. The mixture was concentrated to give a powder, which was subject to chromatograsphy (5-10% methanol in chloroform, silica gel) to give the title compound (1.83 g, 77% yield) as a bright yellow solid.

¹H NMR δ (CD₃SOCD₃): 9.13 (1H, s), 8.93 (1H, t, J=4.7 Hz), 8.42 (1H, d, J=8.6 Hz), 8.04 (1H, d, J=2.7 Hz), 7.94 (1H, d, J=9.0 Hz), 7.68 (1H, dd, J=9.0, 2.7 Hz), 6.76 (1H, d, J=9.0 Hz), 3.39-3.33 (4H, m), 2.55 (4H, q, J=7.0 Hz), 2.32 (3H, s), 1.01 (6H, t, J=7.0 Hz). ¹³C NMR δ(CD₃SOCD₃): 177.40, 170.05, 149.97, 148.35, 136.09, 133.14, 133.08, 131.06, 129.95, 128.29, 126.51, 120.51, 118.35, 107.50, 102.75, 51.80, 47.30, 41.04, 21.70, 12.40. LRMS: m/z=393.0 (M+H)
Compound (VIIB): Octanoic acid 5-(2-diethylamino-ethylamino)-6-oxo-6H-2,10b-diaza-aceanthrylen-8-yl ester

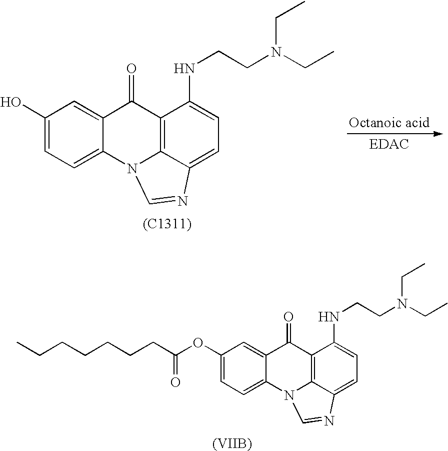

To a stirred solution of C1311 free base (3.50 g, 10 mmol), octanoic acid (1.59 mL, 10 mmol), and N-(3-dimethylaminopropyl)-N′-ethylcarbodiimide hydrochloride (2.30 g, 12 mmol) in DMF (60 mL) were added 4-(dimethylamino)pyridine (12 mg, 0.10 mmol) and triethylamine (2.83 mL, 20 mmol). The reaction mixture was stirred at ambient temperature for 66 hours (over weekend) and then concentrated to yield a solid, which was purified by chromatography 6-20% methanol in chloroform, silica gel) furnished the title compound (4.25 g, 89% yield) as greenish yellow solid.

¹H NMR δ (CD₃SOCD₃): 9.14 (1H, s), 9.07 (1H, s), 8.43 (1H, d, J=8.6 Hz), 8.23 (1H, d, J=9.0 Hz), 8.02 (1H, d, J=2.3 Hz), 7.69 (1H, dd, J=9.0, 2.3 Hz), 6.76 (1H, d, J=9.0 Hz), 3.63-3.54 (2H, m), 3.40-3.36 (2H, m), 3.17-3.11 (2H, m), 2.70 (2H, t, J=6.2 Hz), 2.4-1.4 (12H, m), 1.00 (6H, t, J=7.8 Hz), 0.83 (3H, t, J=7.1 Hz). LRMS: m/z=477.0 (M+H)
Compound (VIIIB): 2,2-Dimethyl-propionic acid 5-(2-diethylamino-ethylamino)-6-oxo-6H-2,10b-diaza-aceanthrylen-8-yl ester

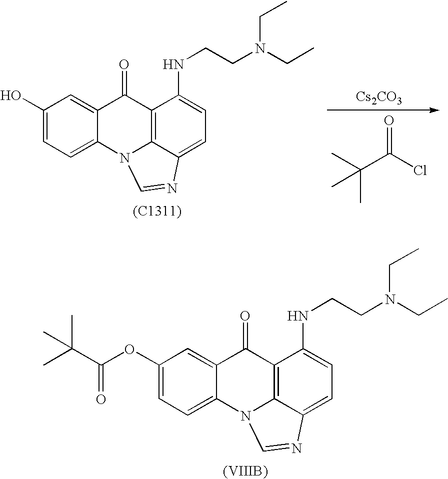

To a stirred solution of C1311 free base (0.280 g, 0.800 mmol) in anhydrous DMF (8 mL) was added cesium carbonate (0.67 g, 2.40 mmol). After 10 minutes, the initial yellow solution became orange. Trimethylacetyl chloride (0.295 mL, 2.40 mmol) was added via a syringe, the orange solution immediately turned greenish and cloudy. After 21 hours, the mixture was concentrated, the resulting solid was subject to chromatography (5-20% methanol in chloroform, silica gel) to give the title compound (0.13 g, 37% yield) as a yellow solid.

¹H NMR δ (CD₃SOCD₃): 9.15 (1H, s), 8.94 (1H, t, J=4.7 Hz), 8.45 (1H, d, J=9.0 Hz), 7.98 (1H, d, J=2.7 Hz), 7.95 (1H, d, J=8.6 Hz), 7.68 (1H, dd, J=9.0, 2.7 Hz), 6.77 (1H, d, J=9.0 Hz), 3.39 (2H, m), 3.30 (2H, m), 2.55 (4H, m), 1.35 (9H, s), 1.02 (6H, t, J=7.4 Hz). ¹³C NMR δ (CD₃SOCD₃): 177.36, 177.15, 149.96, 148.56, 136.09, 133.15, 133.06, 131.07, 129.95, 128.21, 126.52, 120.26, 118.40, 107.60, 102.75, 51.25, 46.97, 41.04, 39.33, 27.45, 12.51. LRMS: m/z=435.3 (M+H)
Compound (IXB): Cyclopropanecarboxylic acid 5-(2-diethylamino-ethylamino)-6-oxo-6H-2,10b-diaza-aceanthrylen-8-yl ester

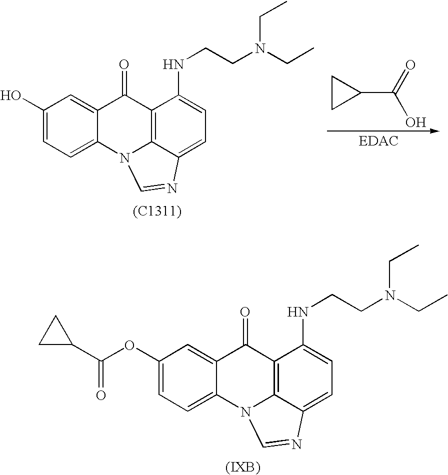

A solution of C1311 free base (1.59 g, 4.54 mmol), N-(3-dimethylaminopropyl)-N′-ethylcarbodiimide hydrochloride (0.96 g, 5.00 mmol), 4-(dimethylamino)pyridine (12 mg, 0.10 mmol), cyclopropanecarboxylic acid (0.360 mL, 4.54 mmol), and triethylamine (1.41 mL, 10 mmol) in anhydrous DMF (25 mL) was stirred at room temperature for 19 hours. The mixture was concentrated to give a powder, which was subject to chromatograsphy (10-25% methanol in chloroform, silica gel) to give the title compound (0.62 g, 32% yield) as a yellow solid. ¹H NMR δ (CD₃SOCD₃): 9.13 (1H, s), 8.89 (1H, t, J=5.1 Hz), 8.42 (1H, d, J=9.0 Hz), 7.98 (1H, d, J=2.3 Hz), 7.93 (1H, d, J=9.0 Hz), 7.69 (1H, dd, J=8.6, 2.3 Hz), 6.78 (1H, d, J=9.0 Hz), 3.9-3.0 (8H, m), 2.81 (1H, m), 2.65 (2H, m), 2.49-2.44 (2H, m), 1.04 (6H, t, J=7.0 Hz). LRMS: m/z=419.2 (M+H)

### Compound (XB): Hexanoic acid 5-(2-diethylamino-ethylamino)-6-oxo-6H-2,10b-diaza-aceanthrylen-8-yl ester

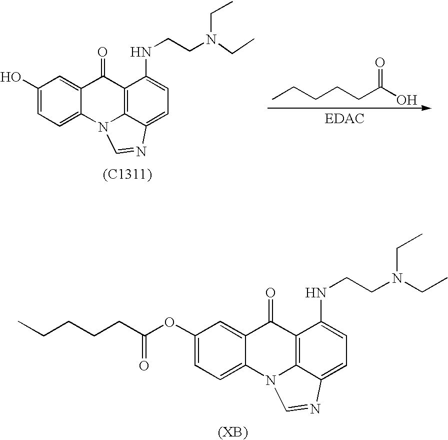

A solution of C1311 free base (2.20 g, 6.28 mmol), N-(3-dimethylaminopropyl)-N′-ethylcarbodiimide hydrochloride (1.33 g, 6.91 mmol), 4-(dimethylamino)pyridine (12 mg, 0.10 mmol), hexanoic acid (0.866 mL, 6.91 mmol), and triethylamine (1.94 mL, 13.8 mmol) in anhydrous DMF (50 mL) was stirred at room temperature for 19 hours. The mixture was concentrated to give a powder, which was subject to chromatograsphy (10-20% methanol in chloroform, silica gel) to give the title compound (0.936 g, 30% yield) as a yellow solid.

### Compound (XIB). Butyric acid 5-(2-diethylamino-ethylamino)-6-oxo-6H-2,10b-diaza-aceanthrylen-8-yl ester

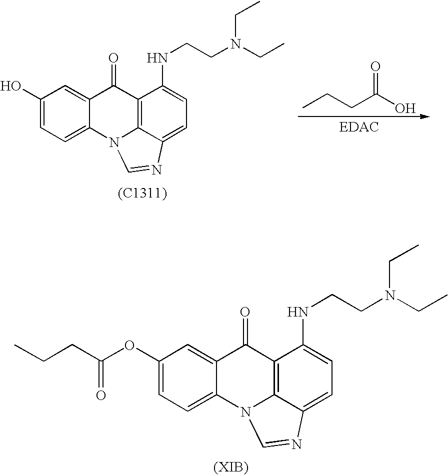

To a stirred solution of C1311 free base (3.01 g, 8.60 mmol) and butyric acid (0.870 mL, 9.46 mmol) in anhydrous DMF (36 mL) were added N-(3-dimethylaminopropyl)-N′-ethylcarbodiimide hydrochloride (1.81 g, 9.46 mmol), 4-(dimethylamino)pyridine (12 mg, 0.10 mmol), and triethylamine (4.21 mL, 30 mmol). The reaction mixture was stirred at RT for 65 hours (over weekend), and concentrated on a rotary evaporator. The resulting residue was purified by chromatography (10-20% methanol in chloroform, silica gel) to afford the title compound (2.25 g, 62% yield) as a yellow solid.

### Example 11

### Ester and Carbonate Compounds of Formula (IA) Selectively Target FLT3 Protein Tyrosine Kinase In Vitro

EC50 and E1000 values for selected compounds of formula (IB) were measured according to the protocol described above in Example 9.

When tested in in vitro dose response assays against FLT3, compounds (VB), (VIB) and (VIIB) exhibited the following values of EC50 and E1000:

| Structure | EC50 | E1000 |
|---|---|---|
| 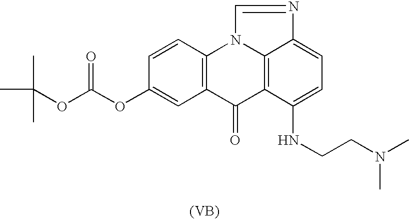 | 96 | 92 |
| 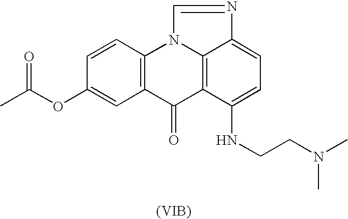 | 471 | 64 |
| 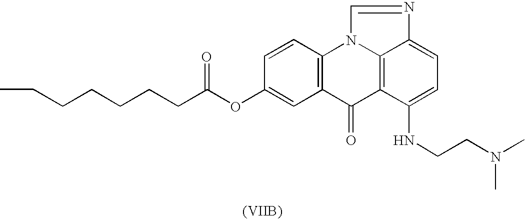 | 277 | 74 |

While this invention has been particularly shown and described with references to preferred embodiments thereof, it will be understood by those skilled in the art that various changes in form and details may be made therein without departing from the scope of the invention encompassed by the appended claims.

1. A compound of formula (I) or a pharmaceutically acceptable salt thereof: 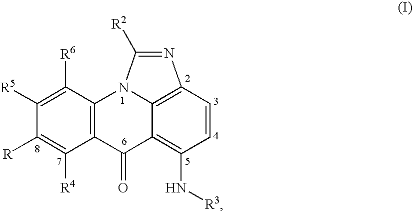
wherein:
R is R^{x}, a hydrolysable group, or alone or taken together with R⁴, or alternatively R⁵, and the intervening carbon atoms is a phenol isosteric group,
wherein R^{x} is —H, an optionally substituted alkyl, hydroxyl, alkoxy group,
a halogen, or a group represented by the following structural formula: 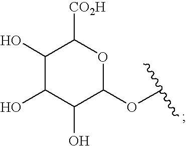
or, R and R⁵ taken together with their intervening carbon atoms form a 5, 6 or 7 member, optionally substituted, cycloalkyl or non-aromatic heterocycle;
or R and R⁴ taken together with their intervening carbon atoms form a 5, 6 or 7 member, optionally substituted, cycloalkyl or non-aromatic heterocycle;
R² is —H, an optionally substituted C1-C10 alkyl or an optionally substituted aryl or aralkyl or heteroaryl;
R³ is —(CH₂), —NR^{a}R^{b}, wherein n is an integer from 1 to 5, and R^{a} and R^{b}, each independently are hydrogen or an optionally substituted alkyl, or R^{a} and R^{b}, taken together with the nitrogen to which they are attached, form group R^{y},
wherein R^{y} is a heteroaryl or a non-aromatic heterocycle, each optionally substituted at one or more substitutable carbon atoms with methyl, hydroxyl, or methoxy, and optionally N′-substituted with C1-C4 alkyl or C1-C4 alkyl substituted with —NR^{c}R^{d}, wherein R^{c} and R^{d} are individually H, methyl or ethyl; and
R⁴, R⁵ and R⁶, are each independently —H, —OH, a halogen or an optionally substituted C1-C6 alkoxy; or
R⁵ and R⁶ taken together with their intervening carbon atoms, form a 5, 6 or 7 member, optionally substituted cycloalkyl or non-aromatic heterocycle,
provided that when R is R^{x}, then R^{a} and R^{b} are not H or optionally substituted alkyl and R^{y} is not optionally substituted N-morpholinyl, optionally substituted N-piperazinyl, or optionally substituted N-pyrazinyl.

2. The compound of claim 1, wherein R is a hydrolysable group selected from groups (II)-(VII): 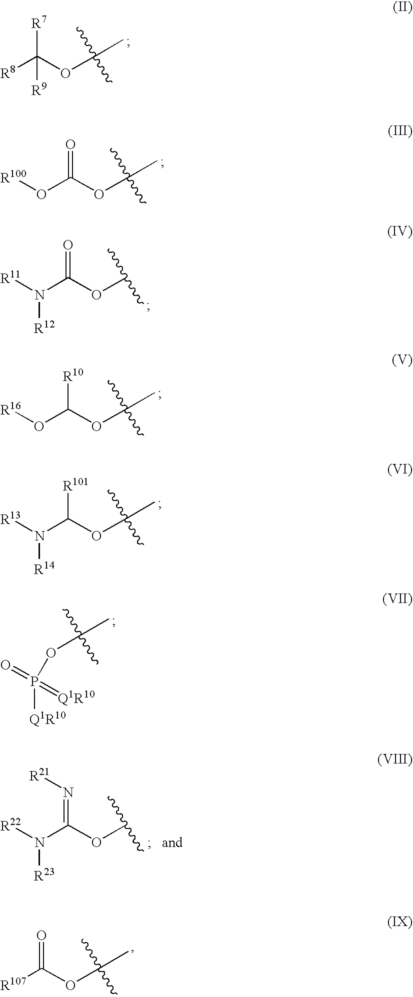
wherein
R⁷ and R⁸ are independently each H, optionally substituted C1-C6 alkyl, or optionally substituted aryl or aralkyl;
R⁹ is carboxyl, carboxamide optionally N-substituted with C1-C4 alkyl, C1-C6 optionally substituted alkanoyl, C1-C6 carbalkoxy, or optionally substituted aroyl;
R¹⁰ is H, C1-C6 optionally substituted alkyl or optionally substituted aryl or aralkyl;
R¹¹ and R¹² are independently each H, optionally substituted C1-C6 alkyl or, taken together with the atom to which they are attached, form an optionally substituted non-aromatic heterocycle;
R¹³ and R¹⁴ are each independently H, optionally substituted C1-C6 alkyl, optionally substituted C1-C6 alkanoyl, or optionally substituted aroyl, or, taken together with the atom to which they are attached, form a heteroaryl or non-aromatic optionally substituted heterocycle;
R¹⁶ is optionally substituted C1-C6 alkyl, optionally substituted aryl or aralkyl, C1-C6 alkanoyl, or optionally substituted aroyl;
R²¹ is optionally substituted C1-C10 alkyl, or an optionally substituted aryl or aralkyl or, R²¹ and R²² taken together with their intervening atoms form a 5-7 membered ring;
R²² and R²³ are each independently —H, or optionally substituted C1-C6 alkyl, provided that R²² and R²³ are not simultaneously hydrogen;
R¹⁰⁰ is optionally substituted C1-C6 alkyl or optionally substituted aryl or aralkyl;
R¹⁰¹ is H, optionally substituted C1-C6 alkyl or optionally substituted aryl or aralkyl;
R¹⁰⁷ is optionally substituted C1-C6 alkyl, optionally substituted aryl or aralkyl, or a non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}; and
Q¹ is O or NH.

3. The compound of claim 2, wherein
R¹⁰ is H, C1-C4 optionally substituted alkyl, phenyl or benzyl;
R¹¹ and R¹² are independently each a H, methyl or ethyl or, taken together with the atom to which they are attached form non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d};
R¹⁶ is a C1-C6 optionally substituted alkanoyl;
R¹⁰⁰ is a C1-C4 optionally substituted alkyl; and
group (VI) is represented by structural formulas (VIa) or (VIb) 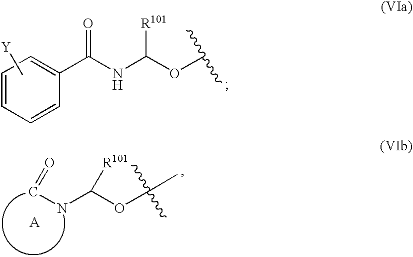
wherein
Y is a halogen, —NO₂, —NH₂, —COOH, alkyl, C1-C3 carbalkoxy, C1-C3 alkoxy group, C1-C3 haloalkyl or C1-C3 haloalkoxy;
ring A is a 5-7 membered non-aromatic heterocycle optionally substituted at one or more substitutable ring carbon atoms with methyl, hydroxyl, oxy, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl; and
R¹⁰¹ is H or C1-C4 alkyl.

4. The compound of claim 3, wherein:
R⁷ and R⁸ are each independently H or optionally substituted C1-C4 alkyl and R⁹ is carboxyl, carboxamide optionally N-substituted with a C1-C4 alkyl, C1-C4 alkanoyl, or C1-C4 carbalkoxy;
N, R¹¹ and R¹², taken together form N-pyrrolidinyl, N-piperidinyl, N-morpholinyl, N-thiomorpholinyl or N-piperazinyl, optionally N′-substituted with C1-C4 alkyl or C1-C4 alkyl substituted with —NR^{c}R^{d}, wherein R^{c} and R^{d} are individually H, methyl or ethyl;
R¹⁰¹ is H, methyl or ethyl;
R¹⁶ is R¹⁶¹—C(O)—, wherein R¹⁶¹ is a branched C3-C6 alkyl; and
R¹⁰⁷ is C1-C6 alkyl optionally substituted with —OH, —SH, halogen, cyano, nitro, amino, —COOH, a C1-C3 alkyl, C1-C3 haloalkyl, C1-C3 alkoxy, C1-C3 haloalkoxy or C1-C3 alkyl sulfanyl, or —(CH₂)ₚ—(CH₂)_{q}—C(O)OH, wherein and q are independently an integer from 1 to 6.

5. The compound of claim 4, wherein
R²¹ is optionally substituted C1-C10 alkyl, phenyl or benzyl optionally substituted with a halogen, —NO₂, —NH₂, —COOH, alkyl, C1-C3 carbalkoxy, C1-C3 alkoxy group, C1-C3 haloalkyl or C1-C3 haloalkoxy; and
R²² and R²³ are each independently —H, or a C1-C3 alkyl.

6. The compound of claim 4, wherein R²¹ and R²², taken together with their intervening atoms, form a 5 or 6 membered non-aromatic heterocycle and R²³ is —H, or a C1-C3 alkyl.

7. The compound of claim 4, wherein R² is —H, C1-C4 alkyl or C1-C4 haloalkyl, R⁴, R⁵ and R⁶ are each independently —H, —OH, C1-C4 alkyl or C1-C4 haloalkyl, or R⁵ and R⁶ taken together are methylenedioxy.

8. The compound of claim 7, wherein
R⁷ and R⁸ are each independently H, methyl or ethyl, and R⁹ is a C1-C4 alkanoyl;
R¹⁰ is a H, C1-C4 alkyl; and
ring A is selected from 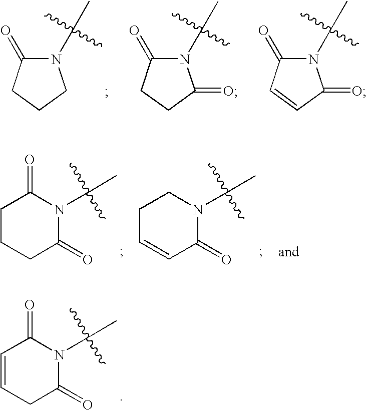
R¹⁰⁷ is C1-C6 alkyl or C1-C6 carboxyalkyl.

9. The compound of claim 1, wherein R alone or taken together with R⁴, or alternatively R⁵, and the intervening carbon atoms is a phenol isosteric group.

10. The compound of claim 9, wherein R alone or taken together with R⁴, or alternatively R⁵, and the intervening carbon atoms is selected from: 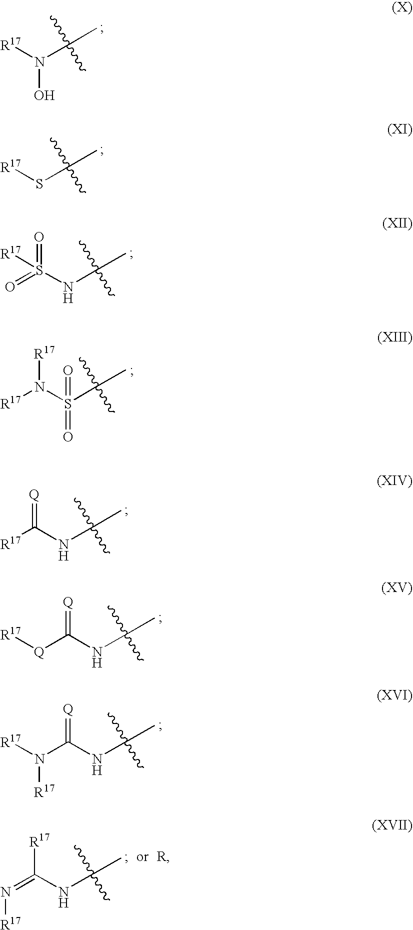
taken together with R⁴ and their intervening atoms are: 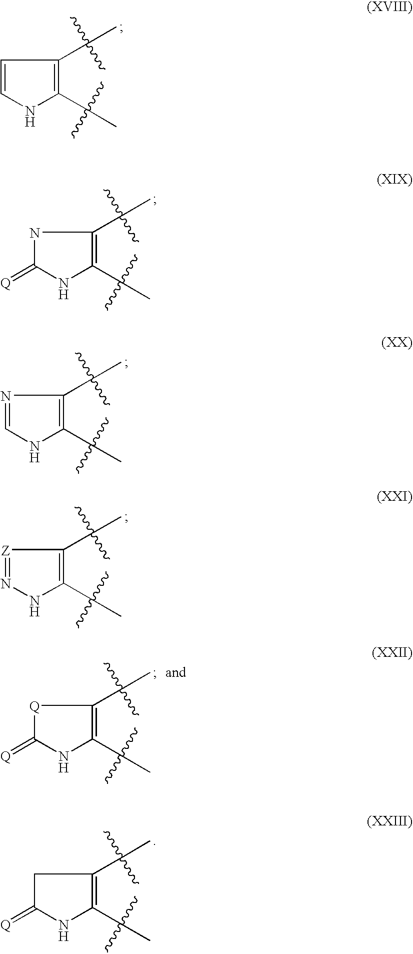
wherein
R¹⁷ for each occurrence, is H, optionally substituted C1-C6 alkyl, optionally substituted aryl or aralkyl or heteroaryl, C1-C6 alkoxyalkyl, optionally substituted aryloxy, aralkyloxy or heteroaryloxy;
Q, for each occurrence, is O or S; and
Z is CH or N.

11. The compound of claim 10, wherein R² is —H, C1-C4 alkyl or C1-C4 haloalkyl, R⁵ and R⁶ are each independently —H, —OH, C1-C4 alkyl or C1-C4 haloalkyl or R⁵ and R⁶ taken together are methylenedioxy.

12. The compounds of claim 11, wherein R¹⁷ is H, optionally substituted C1-C6 alkyl, C1-C6 alkoxyalkyl, or phenyl, benzyl, phenyloxy or benzyloxy, optionally substituted with one or more halogen atoms, —NO₂, —NH₂, —COOH, C1-C3 alkyl, C1-C3 carbalkoxy, C1-C3 alkoxy group, C1-C3 haloalkyl or C1-C3 haloalkoxy.

13. The compounds of claim 12, wherein R¹⁷ is H, C1-C4 alkyl, or phenyl, optionally substituted with one or more halogen atoms or C1-C3 haloalkyls.

14. The compounds of claim 13, wherein R¹⁷ is H, C1-C4 haloalkyl, or phenyl, optionally substituted with one or more C1-C3 haloalkyls.

15. The compounds of claim 14, wherein R¹⁷ is H, trifluoromethyl or phenyl substituted with one or more trifluoromethyls.

16. The compound of claim 1, wherein R^{a} and R^{b}, taken together with the nitrogen
to which they are attached, form group R^{y}.

17. The compound of claim 16, wherein R^{y} is a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}, wherein R^{c} and R^{d} are individually H, methyl or ethyl.

18. The compound of claim 16, wherein R^{y} is a heteroaryl.

19. The compound of claim 18, wherein R^{y} is a N-pyrazinyl or N-pyridinyl.

20. The compound of claim 19, wherein R² is —H, C1-C4 alkyl or C1-C4 haloalkyl and R⁶ is —H, —OH, C1-C4 alkyl or C1-C4 haloalkyl.

21. The compound of claim 20, wherein R^{y} is selected form a group consisting of 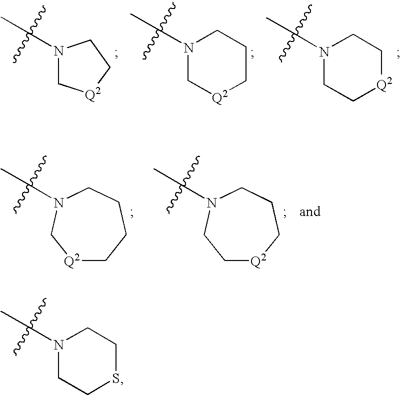
wherein Q² is CH₂, NH, or NR¹⁰², wherein R¹⁰² is methyl or ethyl.

22. The compound of claim 1, wherein R a hydrolysable group, or R alone or taken together with R⁴, or alternatively R⁵, and the intervening carbon atoms is a phenol isosteric group.

23. The compound of claim 22, wherein R is a hydrolysable group.

24. The compound of claim 23, wherein R is selected from groups (II)-(VII): 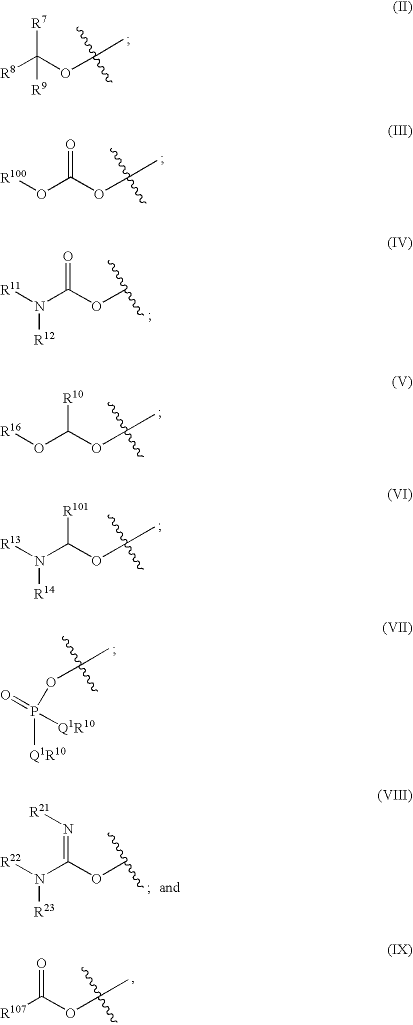
wherein
R⁷ and R⁸ are independently each H, optionally substituted C1-C6 alkyl, optionally substituted aryl or aralkyl;
R⁹ is carboxyl, carboxamide optionally N-substituted with C1-C4 alkyl, C1-C6 alkanoyl, C1-C6 carbalkoxy, or optionally substituted aroyl;
R¹⁰ is H, optionally substituted C1-C6 alkyl or optionally substituted aryl or aralkyl;
R¹¹ and R¹² are independently each H, optionally substituted C1-C6 alkyl or, taken together with the atom to which they are attached, form an optionally substituted non-aromatic heterocycle;
R¹³ and R¹⁴ are each independently H, optionally substituted C1-C6 alkyl, optionally substituted C1-C6 alkanoyl, or optionally substituted aroyl, or, taken together with the atom to which they are attached, form an optionally substituted heteroaryl or non-aromatic optionally substituted heterocycle;
R¹⁶ is optionally substituted C1-C6 alkyl, optionally substituted aryl or aralkyl, C1-C6 alkanoyl, or optionally substituted aroyl;
R²¹ is optionally substituted C1-C10 alkyl, or an optionally substituted aryl or aralkyl or, R²¹ and R²² taken together with their intervening atoms form a 5-7 membered non-aromatic heterocycle;
R²² and R²³ are each independently —H, or a optionally substituted C1-C6 alkyl,
provided that R²² and R²³ are not simultaneously hydrogens;
R¹⁰⁰ is optionally substituted C1-C6 alkyl or optionally substituted aryl or aralkyl;
R¹⁰¹ is H, optionally substituted C1-C6 alkyl or optionally substituted aryl or aralkyl;
R¹⁰⁷ is optionally substituted C1-C6 alkyl, optionally substituted aryl or aralkyl, or a non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}; and
Q¹ is O or NH.

25. The compound of claim 24, wherein R^{y} is an optionally substituted heteroaryl.

26. The compound of claim 24, wherein n is an integer from 1 to 5, and R^{a} and R^{b}, each independently are hydrogen or an alkyl, or R^{a} and R^{b}, taken together with the nitrogen to which they are attached, form a 5-7 membered non-aromatic heterocycle optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c}C and R^{d} are individually H, methyl or ethyl.

27. The compound of claim 26, wherein
R⁷ and R⁸ are independently each H, optionally substituted C1-C6 alkyl or phenyl or benzyl, each optionally substituted with one or more hydroxyl, C1-C3 alkoxy, amino, alkylamino, halogen, haloalkyl or haloalkoxy groups; and
R⁹ is carboxyl, carboxamide optionally N-substituted with a C1-C4 alkyl, C1-C4 alkanoyl, or C1-C4 carbalkoxy.

28. The compound of claim 27, wherein
R¹⁰ is H or C1-C4 alkyl, phenyl or benzyl each optionally substituted with one or more hydroxyl, C1-C3 alkoxy, amino, alkylamino, halogen, haloalkyl or haloalkoxy groups;
R¹¹ and R¹² are independently each a H, methyl or ethyl or, taken together with the atom to which they are attached form non-aromatic heterocycle, optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl;
R¹⁶ is a optionally substituted C1-C6 alkanoyl;
R¹⁰⁰ is a C1-C4 alkyl;
R¹⁰⁷ is C1-C6 alkyl optionally substituted with —OH, —SH, halogen, cyano, nitro, amino, —COOH, a C1-C3 alkyl, C1-C3 haloalkyl, C1-C3 alkoxy, C1-C3 haloalkoxy or C1-C3 alkyl sulfanyl, or —(CH₂)ₚ—(CH₂)_{q}—C(O)OH, wherein and q are independently an integer from 1 to 6; and
group (VI) is represented by structural formulas (VIa) or (VIb) 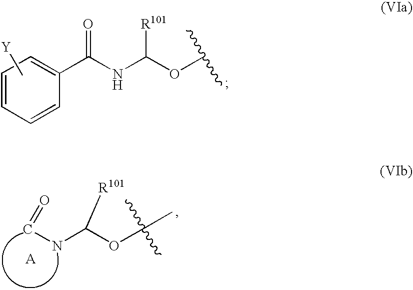
wherein
Y is a halogen, —NO₂, —NH₂, —COOH, alkyl, C1-C3 carbalkoxy, C1-C3 alkoxy group, C1-C3 haloalkyl or C1-C3 haloalkoxy;
ring A is a 5-7 membered non-aromatic heterocycle optionally substituted at one or more substitutable ring carbon atoms with methyl, hydroxyl, oxy, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl; and
R¹⁰¹ is H or C1-C4 alkyl.

29. The compound of claim 28, wherein
either R¹² is optionally substituted C1-C10 alkyl, phenyl or benzyl optionally substituted with a halogen, —NO₂, —NH₂, —COOH, alkyl, C1-C3 carbalkoxy, C1-C3 alkoxy group, C1-C3 haloalkyl or C1-C3 haloalkoxy, and R²² and R²³ are each independently —H, or a C1-C3 alkyl; or
R²¹ and R²², taken together with their intervening atoms, form a 5 or 6 membered non-aromatic heterocycle and R²³ is —H, or a C1-C3 alkyl.

30. The compound of claim 29, wherein R² is —H, C1-C4 alkyl or C1-C4 haloalkyl, R⁴, R⁵ and R⁶ are each independently —H, —OH, C1-C4 alkyl or C1-C4 haloalkyl, or R⁵ and R⁶ taken together are methylenedioxy.

31. The compound of claim 30, wherein
either n is 2 or 3 and R^{a} and R^{b}, is each independently a hydrogen or a C1-C3 alkyl; or
R^{a} and R^{b}, taken together with the nitrogen to which they are attached, form group R^{y} selected form a group consisting of 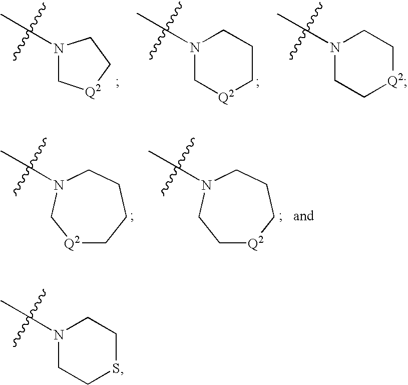
wherein Q2 is CH₂, NH, or NR¹⁰², wherein R¹⁰² methyl or ethyl.

32. The compound of claim 31, wherein:
R⁷ and R⁸ are each independently H, methyl or ethyl, and R⁹ is a C1-C4 alkanoyl;
R¹⁰ is a H, or C1-C4 alkyl;
NR¹¹R¹² is N-pyrrolidinyl, N-piperidinyl, N-morpholinyl, N-thiomorpholinyl or N-piperazinyl, optionally N′-substituted with C1-C4 alkyl or C1-C4 alkyl substituted with —NR^{c}R^{d}, wherein R^{c} and R^{d} are individually H, methyl or ethyl;
R¹⁰¹ is H, methyl or ethyl;
R¹⁶ is a branched C3-C6 alkanoyl; and
R¹⁰⁷ is C1-C6 alkyl or C1-C6 carboxyalkyl.

33. The compound of claim 32, wherein ring A is selected from:

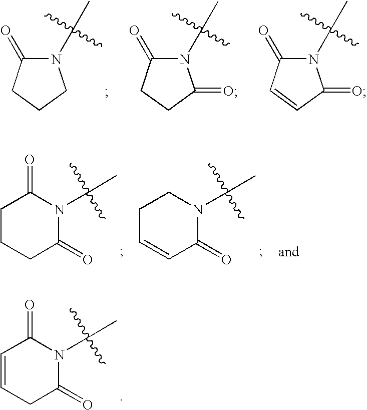

34. The compound of claim 33, wherein R²—H, methyl or ethyl, and R⁶ is —H, —OH or methyl or ethyl.

35. The compound of claim 22, wherein R alone or taken together with R⁴, or alternatively R⁵, and the intervening carbon atoms is the phenol isosteric group.

36. The compound of claim 35, wherein the phenol isosteric group is selected from: 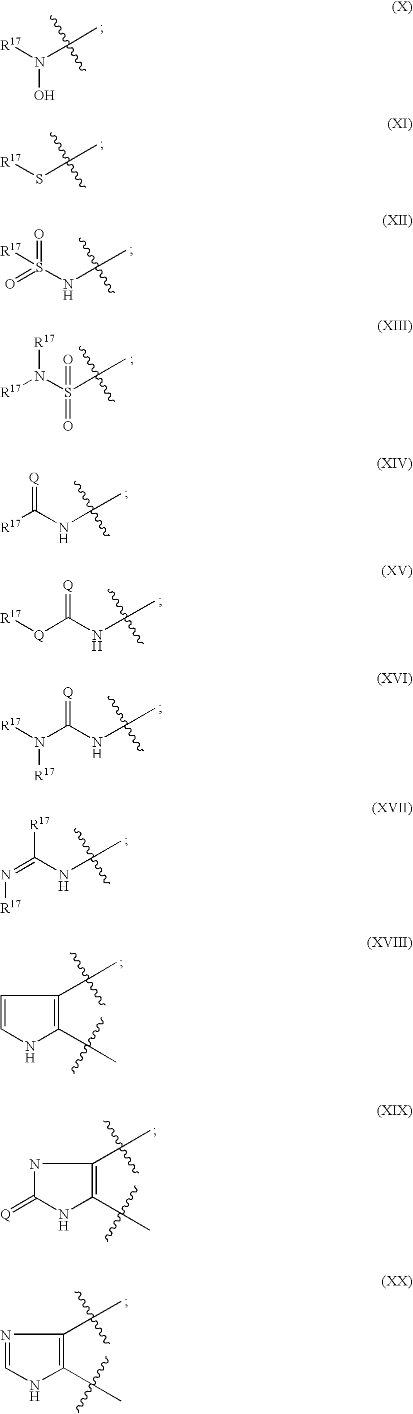 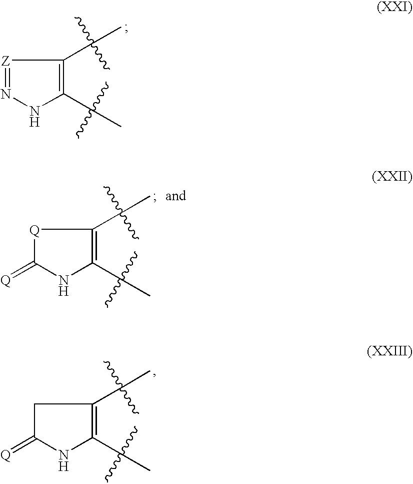
wherein
R¹⁷ for each occurrence, is H, optionally substituted C1-C6 alkyl, optionally substituted aryl or aralkyl or heteroaryl, C1-C6 alkoxyalkyl, optionally substituted aryloxy, aralkyloxy or heteroaryloxy;
Q is O or S; and
Z is CH or N.

37. The compound of claim 36, wherein R^{y} is an optionally substituted heteroaryl.

38. The compound of claim 36, wherein n is an integer from 1 to 5, and R^{a} and R^{b}, each independently are hydrogen or an alkyl, or R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}, wherein R^{c} and R^{d} are individually H, methyl or ethyl.

39. The compound of claim 38, wherein R² is —H, C1-C4 alkyl or C1-C4 haloalkyl, and R⁵ and R⁶ are each independently —H, —OH, C1-C4 alkyl or C1-C4 haloalkyl, or taken together are methylenedioxy.

40. The compounds of claim 39, wherein R¹⁷ is H, optionally substituted C1-C6 alkyl, or C1-C6 alkoxyalkyl, or phenyl, benzyl, phenyloxy or benzyloxy each optionally substituted with halogen, —NO₂, —NH₂, —COOH, C1-C3 alkyl, C1-C3 carbalkoxy, C1-C3 a alkoxy group, C1-C3 haloalkyl or C1-C3 haloalkoxy.

41. The compound of claim 40, wherein
either n is 2 or 3 and R^{a} and R^{b}, is each independently a hydrogen or a C1-C3 alkyl; or
R^{a} and R^{b}, taken together with the nitrogen to which they are attached, form group R¹ selected form a group consisting of 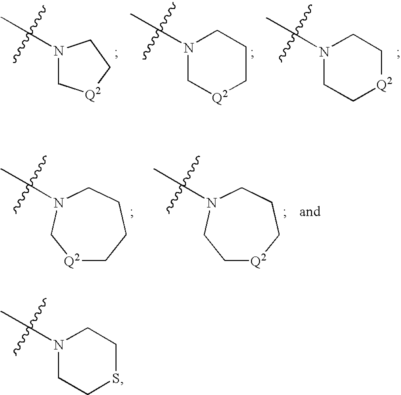
wherein Q² is CH₂, NH, or NR¹⁰², wherein R¹⁰² is methyl or ethyl.

42. The compounds of claim 41, wherein R¹⁷ is H, C1-C4 alkyl, or phenyl, optionally substituted with one or more halogen atoms, —NO₂, —NH₂, —COOH, C1-C3 alkyl, C1-C3 carbalkoxy, C1-C3 a alkoxy group, C1-C3 haloalkyl or C1-C3 haloalkoxy.

43. The compounds of claim 42, wherein R¹⁷ is H, C1-C4 haloalkyl or phenyl optionally substituted with one or more halogen atoms or C1-C3 haloalkyls.

44. The compound of claim 43, wherein R² is —H, methyl or ethyl and R⁶ is —H, —OH, methyl, ethyl.

45. The compounds of claim 44, wherein R¹⁷ is H, trifluoromethyl or phenyl substituted with one or more trifluoromethyls.

46. A method of treating an inflammatory or a demyelinating disorder in a patient, comprising administering to said patient a therapeutically effective amount of a compound of formula (I) or a pharmaceutically acceptable salt thereof: 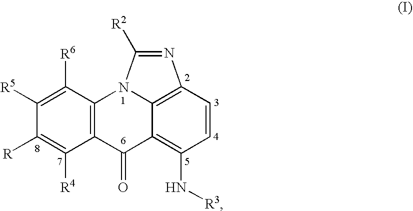
wherein:
R is R^{x}, a hydrolysable group, or alone or taken together with R⁴, or alternatively R⁵, and the intervening carbon atoms is a phenol isosteric group,
wherein R^{x} is —H, an optionally substituted alkyl, hydroxyl, alkoxy group,
a halogen, or a group represented by the following structural formula: 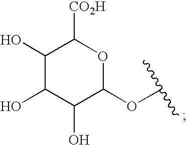
or, R and R⁵ taken together with their intervening carbon atoms form a 5, 6 or 7 member, optionally substituted, cycloalkyl or non-aromatic heterocycle;
or R and R⁴ taken together with their intervening carbon atoms form a 5, 6 or 7 member, optionally substituted, cycloalkyl or non-aromatic heterocycle;
R² is —H, an optionally substituted optionally substituted C1-C10 alkyl or an optionally substituted aryl or aralkyl or heteroaryl;
R³ is —(CH₂), —NR^{a}R^{b}, wherein n is an integer from 1 to 5, and R^{a} and R^{b}, each independently are hydrogen or an optionally substituted alkyl, or R^{a} and R^{b}, taken together with the nitrogen to which they are attached, form group R^{y},
wherein R^{y} is a heteroaryl or a non-aromatic heterocycle, each optionally substituted at one or more substitutable carbon atoms with methyl, hydroxyl, or methoxy, and optionally N′-substituted with C1-C4 alkyl or
C1-C4 alkyl substituted with —NR^{c}R^{d}, wherein R^{c} and R^{d} are individually H, methyl or ethyl; and
R⁴, R⁵ and R⁶, are each independently —H, —OH, a halogen or optionally substituted C1-C6 alkoxy; or
R⁵ and R⁶ taken together with their intervening carbon atoms, form a 5, 6 or 7 member, optionally substituted cycloalkyl or non-aromatic heterocycle,
provided that when R is R^{x}, then R^{a} and R^{b} are not H or optionally substituted alkyl and R^{y} is not optionally substituted N-morpholinyl, or optionally substituted N-pyrazinyl.

47. The method of claim 46, wherein R is a hydrolysable group selected from groups (II)-(VII): 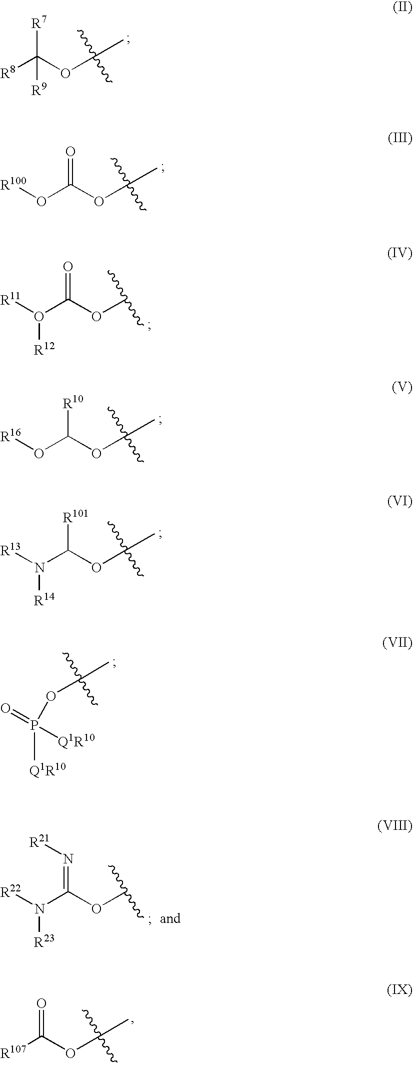
wherein
R⁷ and R⁸ are independently each H, optionally substituted C1-C6 alkyl, or optionally substituted aryl or aralkyl;
R⁹ is carboxyl, carboxamide optionally N-substituted with C1-C4 alkyl, optionally substituted C1-C6 alkanoyl, optionally substituted C1-C6 carbalkoxy, or optionally substituted aroyl;
R¹⁰ is H, optionally substituted C1-C6 alkyl or optionally substituted aryl or aralkyl;
R¹¹ and R¹² are independently each H, optionally substituted C1-C6 alkyl or, taken together with the atom to which they are attached, form an optionally substituted non-aromatic heterocycle;
R¹³ and R¹⁴ are each independently H, optionally substituted C1-C6 alkyl, optionally substituted C1-C6 alkanoyl, or optionally substituted aroyl, or, taken together with the atom to which they are attached, form a heteroaryl or non-aromatic optionally substituted heterocycle;
R¹⁶ is optionally substituted C1-C6 alkyl optionally substituted aryl or aralkyl, optionally substituted C1-C6 alkanoyl, or optionally substituted aroyl;
R²¹ is optionally substituted C1-C10 alkyl or an optionally substituted aryl or aralkyl or, R²¹ and R²² taken together with their intervening atoms form a 5-7 membered ring;
R²² and R²³ are each independently —H, or optionally substituted C1-C6 alkyl, provided that R²² and R²³ are not simultaneously hydrogen;
R¹⁰⁰ is optionally substituted C1-C6 alkyl or optionally substituted aryl or aralkyl;
R¹⁰¹ is H, optionally substituted C1-C6 alkyl or optionally substituted aryl or aralkyl;
R¹⁰⁷ is optionally substituted C1-C6 alkyl, optionally substituted aryl or aralkyl, or a non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}; and
Q¹ is O or NH.

48. The method of claim 47, wherein
R¹⁰ is H, C1-C4 alkyl, phenyl or benzyl;
R¹¹ and R¹² are independently each a H, methyl or ethyl or, taken together with the atom to which they are attached form non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d};
R¹⁶ is a optionally substituted C1-C6 alkanoyl;
R¹⁰⁰ is a C1-C4 alkyl; and
group (VI) is represented by structural formulas (VIa) or (VIb) 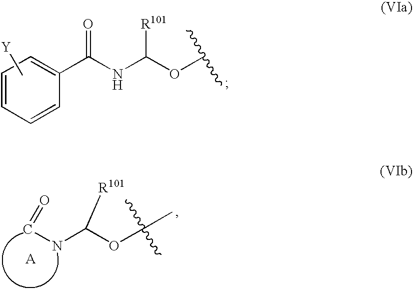
wherein
Y is a halogen, —NO₂, —NH₂, —COOH, alkyl, C1-C3 carbalkoxy, C1-C3 alkoxy group, C1-C3 haloalkyl or C1-C3 haloalkoxy;
ring A is a 5-7 membered non-aromatic heterocycle optionally substituted at one or more substitutable ring carbon atoms with methyl, hydroxyl, oxy, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl; and
R¹⁰¹ is H or C1-C4 alkyl.

49. The method of claim 48, wherein:
R⁷ and R⁸ are each independently H or C1-C4 alkyl and R⁹ is carboxyl, carboxamide optionally N-substituted with a C1-C4 alkyl, C1-C4 alkanoyl, or C1-C4 carbalkoxy;
N, R¹¹ and R¹², taken together form N-pyrrolidinyl, N-piperidinyl, N-morpholinyl, N-thiomorpholinyl or N-piperazinyl, optionally N′-substituted with C1-C4 alkyl or C1-C4 alkyl substituted with —NR^{c}R^{d}, wherein R^{c} and R^{d} are individually H, methyl or ethyl;
R¹⁰¹ is H, methyl or ethyl;
R¹⁶ is R¹⁶¹—C(O)—, wherein R¹⁶¹ is a branched C3-C6 alkyl; and
R¹⁰⁷ is C1-C6 alkyl optionally substituted with —OH, —SH, halogen, cyano, nitro, amino, —COOH, a C1-C3 alkyl, C1-C3 haloalkyl, C1-C3 alkoxy, C1-C3 haloalkoxy or C1-C3 alkyl sulfanyl, or —(CH₂)ₚ—(CH₂)_{q}—C(O)OH, wherein and q are independently an integer from 1 to 6.

50. The method of claim 49, wherein
R²¹ is optionally substituted C1-C10 alkyl, phenyl or benzyl optionally substituted with a halogen, —NO₂, —NH₂, —COOH, alkyl, C1-C3 carbalkoxy, C1-C3 alkoxy group, C1-C3 haloalkyl or C1-C3 haloalkoxy; and
R²² and R²³ are each independently —H, or a C1-C3 alkyl.

51. The method of claim 50, wherein R²¹ and R²², taken together with their intervening atoms, form a 5 or 6 membered non-aromatic heterocycle and R²³ is —H, or a C1-C3 alkyl.

52. The method of claim 50, wherein R² is —H, C1-C4 alkyl or C1-C4 haloalkyl, R⁴, R⁵ and R⁶ are each independently —H, —OH, C1-C4 alkyl or C1-C4 haloalkyl, or R⁵ and R⁶ taken together are methylenedioxy.

53. The method of claim 52, wherein
R⁷ and R⁸ are each independently H, methyl or ethyl, and R⁹ is a C1-C4 alkanoyl;
R¹⁰ is a H, C1-C4 alkyl; and
ring A is selected from 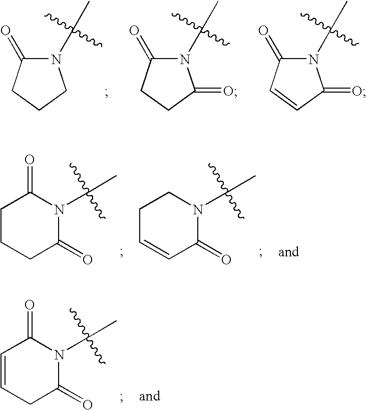
R¹⁰⁷ is C1-C6 alkyl or C1-C6 carboxyalkyl.

54. The method of claim 46, wherein R alone or taken together with R⁴, or alternatively R⁵, and the intervening carbon atoms is a phenol isosteric group.

55. The method of claim 54, wherein R alone or taken together with R⁴, or alternatively R⁵, and the intervening carbon atoms is selected from: 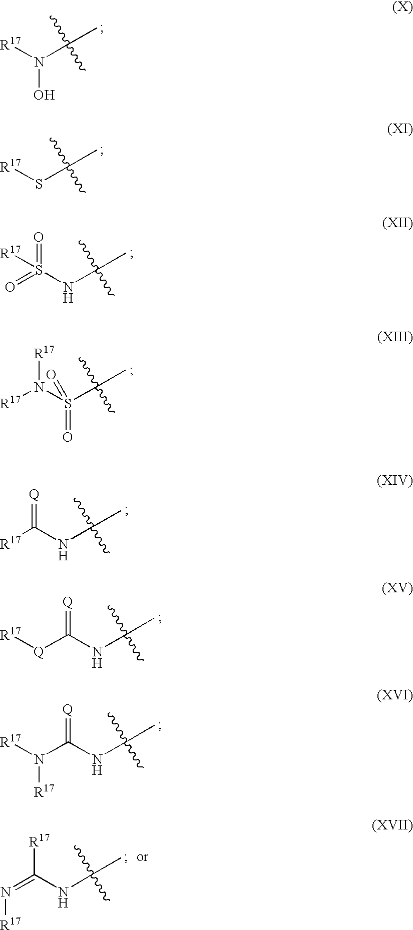
R, taken together with R⁴ and their intervening atoms are: 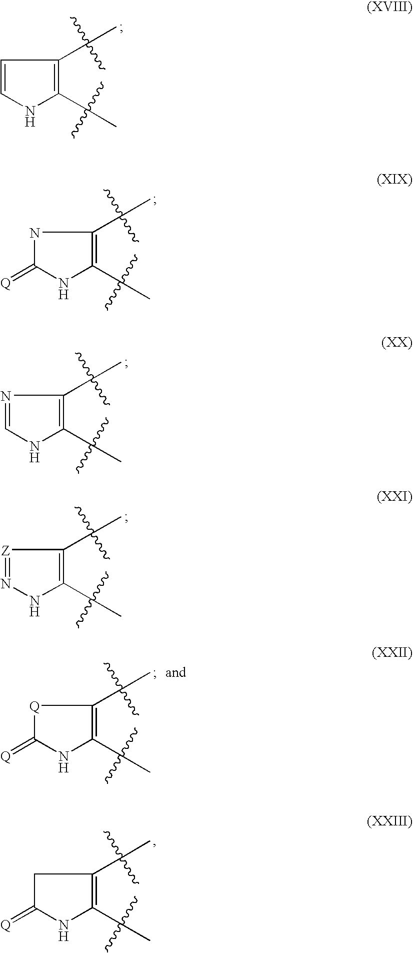
wherein
R¹⁷ for each occurrence, is H, optionally substituted C1-C6 alkyl, optionally substituted aryl or aralkyl or heteroaryl, C1-C6 alkoxyalkyl, optionally substituted aryloxy, aralkyloxy or heteroaryloxy;
Q, for each occurrence, is O or S; and
Z is CH or N.

56. The method of claim 55, wherein R² is —H, C1-C4 alkyl or C1-C4 haloalkyl, R⁵ and R⁶ are each independently —H, —OH, C1-C4 alkyl or C1-C4 haloalkyl or R⁵ and R⁶ taken together are methylenedioxy.

57. The method of claim 56, wherein R¹⁷ is H, optionally substituted C1-C6 alkyl, C1-C6 alkoxyalkyl, or phenyl, benzyl, phenyloxy or benzyloxy, optionally substituted with one or more halogen atoms, —NO₂, —NH₂, —COOH, C1-C3 alkyl, C1-C3 carbalkoxy, C1-C3 alkoxy group, C1-C3 haloalkyl or C1-C3 haloalkoxy.

58. The method of claim 57, wherein R¹⁷ is H, C1-C4 alkyl, or phenyl, optionally substituted with one or more halogen atoms or C1-C3 haloalkyls.

59. The method of claim 58, wherein R¹⁷ is H, C1-C4 haloalkyl, or phenyl, optionally substituted with one or more C1-C3 haloalkyls.

60. The method of claim 59, wherein R¹⁷ is H, trifluoromethyl or phenyl substituted with one or more trifluoromethyls.

61. The method of claim 46, wherein R^{a} and R^{b}, taken together with the nitrogen to which they are attached, form group R′.

62. The method of claim 61, wherein R^{y} is a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}, wherein R^{c} and R^{d} are individually H, methyl or ethyl.

63. The method of claim 61, wherein R^{y} is a heteroaryl.

64. The method of claim 63, wherein R^{y} is a N-pyrazinyl or N-pyridinyl.

65. The method of claim 62, wherein R² is —H, C1-C4 alkyl or C1-C4 haloalkyl and R⁶ is —H, —OH, C1-C4 alkyl or C1-C4 haloalkyl.

66. The method of claim 65, wherein R^{y} is selected form a group consisting of 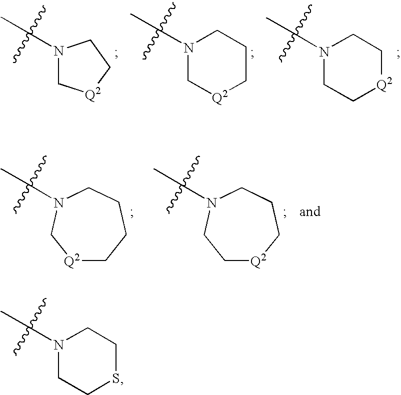
wherein Q² is CH₂, NH, or NR¹⁰², wherein R¹⁰² is methyl or ethyl.

67. The method of claim 46, wherein
R a hydrolysable group, or R alone or taken together with R⁴, or alternatively R⁵, and the intervening carbon atoms is a phenol isosteric group.

68. The method of claim 67, wherein R is a hydrolysable group.

69. The method of claim 68, wherein R is selected from groups (II)-(VII): 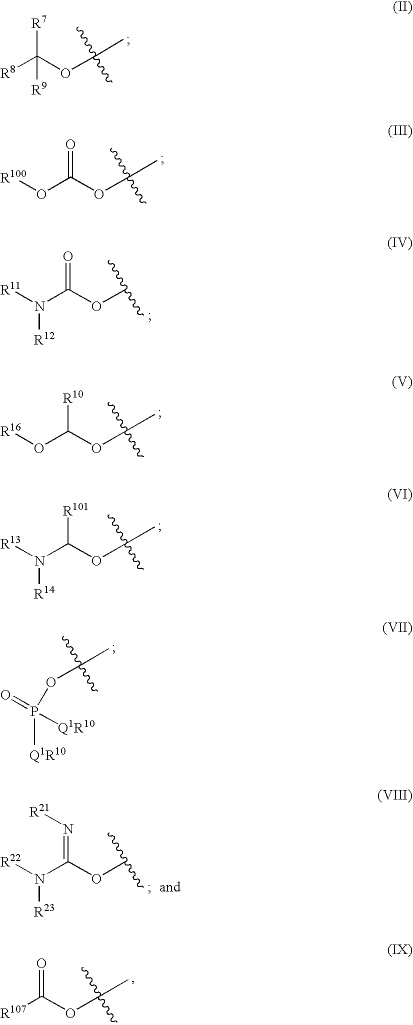
wherein
R⁷ and R⁸ are independently each H, optionally substituted C1-C6 alkyl, optionally substituted aryl or aralkyl;
R⁹ is carboxyl, carboxamide optionally N-substituted with C1-C4 alkyl, optionally substituted C1-C6 alkanoyl, optionally substituted C1-C6 carbalkoxy, or optionally substituted aroyl;
R¹⁰ is H, optionally substituted C1-C6 alkyl or optionally substituted aryl or aralkyl;
R¹¹ and R¹² are independently each H, optionally substituted C1-C6 alkyl or, taken together with the atom to which they are attached, form an optionally substituted non-aromatic heterocycle;
R¹³ and R¹⁴ are each independently H, optionally substituted C1-C6 alkyl, optionally substituted C1-C6 alkanoyl, or optionally substituted aroyl, or, taken together with the atom to which they are attached, form an optionally substituted heteroaryl or non-aromatic optionally substituted heterocycle;
R¹⁶ is optionally substituted C1-C6 alkyl, optionally substituted aryl or aralkyl, optionally substituted C1-C6 alkanoyl, or optionally substituted aroyl;
R²¹ is optionally substituted C1-C10 alkyl, or an optionally substituted aryl or aralkyl or, R²¹ and R²² taken together with their intervening atoms form a 5-7 membered non-aromatic heterocycle;
R²² and R²³ are each independently —H, or optionally substituted C1-C6 alkyl, provided that R²² and R²³ are not simultaneously hydrogens;
R¹⁰⁰ is optionally substituted C1-C6 alkyl or optionally substituted aryl or aralkyl;
R¹⁰¹ is H, optionally substituted C1-C6 alkyl or optionally substituted aryl or aralkyl;
R¹⁰⁷ is optionally substituted C1-C6 alkyl, optionally substituted aryl or aralkyl, or a non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}; and
Q¹ is O or NH.

70. The method of claim 69, wherein R^{y} is an optionally substituted heteroaryl.

71. The method of claim 69, wherein n is an integer from 1 to 5, and R^{a} and R^{b}, each independently are hydrogen or an alkyl, or R^{a} and R^{b}, taken together with the nitrogen to which they are attached, form a 5-7 membered non-aromatic heterocycle optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl.

72. The method of claim 71, wherein
R⁷ and R⁸ are independently each H, optionally substituted C1-C6 alkyl or phenyl or benzyl, each optionally substituted with one or more hydroxyl, C1-C3 alkoxy, amino, alkylamino, halogen, haloalkyl or haloalkoxy groups; and
R⁹ is carboxyl, carboxamide optionally N-substituted with a C1-C4 alkyl, C1-C4 alkanoyl, or C1-C4 carbalkoxy.

73. The method of claim 72, wherein
R¹⁰ is H, C1-C4 alkyl, or phenyl or benzyl each optionally substituted with one or more hydroxyl, C1-C3 alkoxy, amino, alkylamino, halogen, haloalkyl or haloalkoxy groups;
R¹¹ and R¹² are independently each a H, methyl or ethyl or, taken together with the atom to which they are attached form non-aromatic heterocycle, optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl;
R¹⁶ is a optionally substituted C1-C6 alkanoyl;
R¹⁰⁰ is a C1-C4 alkyl;
R¹⁰⁷ is C1-C6 alkyl optionally substituted with —OH, —SH, halogen, cyano, nitro, amino, —COOH, a C1-C3 alkyl, C1-C3 haloalkyl, C1-C3 alkoxy, C1-C3 haloalkoxy or C1-C3 alkyl sulfanyl, or —(CH₂)ₚ—(CH₂)_{q}—C(O)OH, wherein and q are independently an integer from 1 to 6; and
group (VI) is represented by structural formulas (VIa) or (VIb) 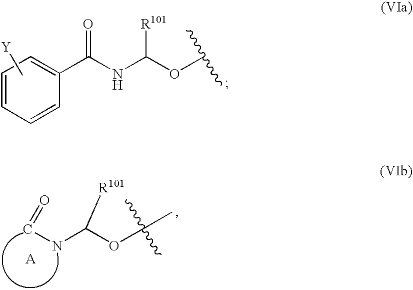
wherein
Y is a halogen, —NO₂, —NH₂, —COOH, alkyl, C1-C3 carbalkoxy, C1-C3 alkoxy group, C1-C3 haloalkyl or C1-C3 haloalkoxy;
ring A is a 5-7 membered non-aromatic heterocycle optionally substituted at one or more substitutable ring carbon atoms with methyl, hydroxyl, oxy, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl; and
R¹⁰¹ is H or C1-C4 alkyl.

74. The method of claim 73, wherein either R²¹ is optionally substituted C1-C10 alkyl, phenyl or benzyl optionally substituted with a halogen, —NO₂, —NH₂, —COOH, alkyl, C1-C3 carbalkoxy, C1-C3 alkoxy group, C1-C3 haloalkyl or C1-C3 haloalkoxy, and R²² and R²³ are each independently —H, or a C1-C3 alkyl; or
R²¹ and R²², taken together with their intervening atoms, form a 5 or 6 membered non-aromatic heterocycle and R²³ is —H, or a C1-C3 alkyl.

75. The method of claim 74, wherein R² is —H, C1-C4 alkyl or C1-C4 haloalkyl, R⁴, R⁵ and R⁶ are each independently —H, —OH, C1-C4 alkyl or C1-C4 haloalkyl, or R⁵ and R⁶ taken together are methylenedioxy.

76. The method of claim 75, wherein either n is 2 or 3 and R^{a} and R^{b}, is each independently a hydrogen or a C1-C3 alkyl; or
R^{a} and R^{b}, taken together with the nitrogen to which they are attached, form group R^{y} selected form a group consisting of 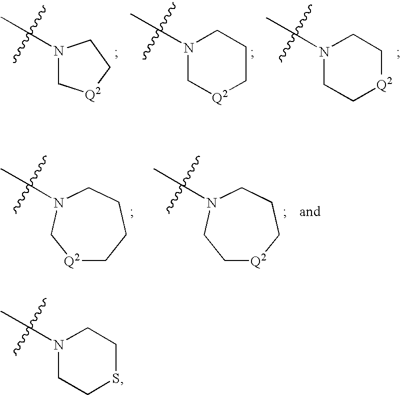
wherein Q² is CH₂, NH, or NR¹⁰², wherein R¹⁰² is methyl or ethyl.

77. The method of claim 76, wherein:
R⁷ and R⁸ are each independently H, methyl or ethyl, and R⁹ is a C1-C4 alkanoyl;
R¹⁰ is a H, or C1-C4 alkyl;
NR¹¹R¹² is N-pyrrolidinyl, N-piperidinyl, N-morpholinyl, N-thiomorpholinyl or N-piperazinyl, optionally N′-substituted with C1-C4 alkyl or C1-C4 alkyl substituted with —NR^{c}R^{d}, wherein R^{c} and R^{d} are individually H, methyl or ethyl;
R¹⁰¹ is H, methyl or ethyl;
R¹⁶ is a branched C3-C6 alkanoyl; and
R¹⁰⁷ is C1-C6 alkyl or C1-C6 carboxyalkyl.

78. The method of claim 77, wherein ring A is selected from:

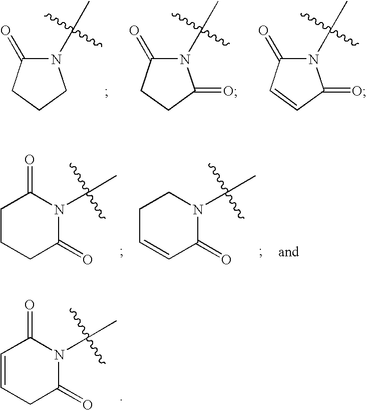

79. The method of claim 78, wherein R²—H, methyl or ethyl, and R⁶ is —H, —OH or methyl or ethyl.

80. The method of claim 67, wherein R alone or taken together with R⁴, or alternatively R⁵, and the intervening carbon atoms is the phenol isosteric group.

81. The method of claim 80, wherein the phenol isosteric group is selected from: 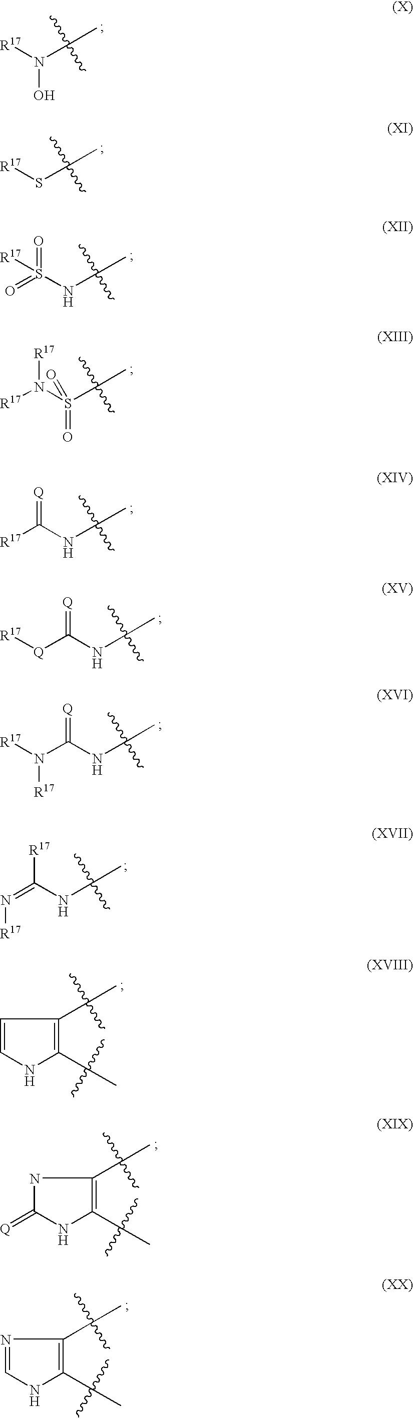 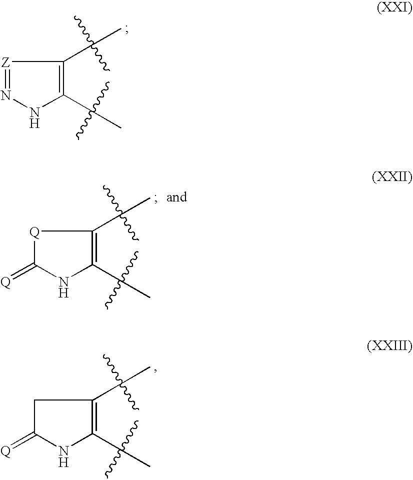
wherein
R¹⁷ for each occurrence, is H, optionally substituted C1-C6 alkyl, optionally substituted aryl or aralkyl or heteroaryl, C1-C6 alkoxyalkyl, optionally substituted aryloxy, aralkyloxy or heteroaryloxy;
Q is O or S; and
Z is CH or N.

82. The method of claim 81, wherein R^{y} is an optionally substituted heteroaryl.

83. The method of claim 81, wherein n is an integer from 1 to 5, and R^{a} and R^{b}, each independently are hydrogen or an alkyl, or R^{a} and R^{b}, taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d}, wherein R^{c} and R^{d} are individually H, methyl or ethyl.

84. The method of claim 83, wherein R² is —H, C1-C4 alkyl or C1-C4 haloalkyl, and R⁵ and R⁶ are each independently —H, —OH, C1-C4 alkyl or C1-C4 haloalkyl, or taken together are methylenedioxy.

85. The method of claim 84, wherein R¹⁷ is H, optionally substituted C1-C6 alkyl, or C1-C6 alkoxyalkyl, or phenyl, benzyl, phenyloxy or benzyloxy each optionally substituted with halogen, —NO₂, —NH₂, —COOH, C1-C3 alkyl, C1-C3 carbalkoxy, C1-C3 a alkoxy group, C1-C3 haloalkyl or C1-C3 haloalkoxy.

86. The method of claim 85, wherein either n is 2 or 3 and R^{a} and R^{b}, is each independently a hydrogen or a C1-C3 alkyl; or
R^{a} and R^{b}, taken together with the nitrogen to which they are attached, form group R^{y} selected form a group consisting of 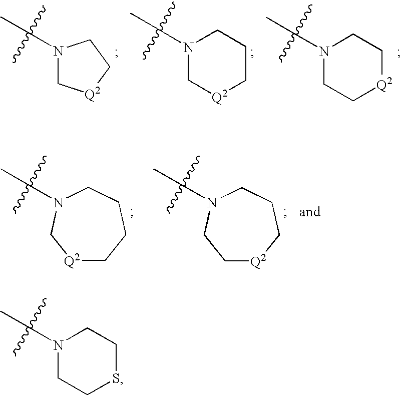
wherein Q² is CH₂, NH, or NR¹⁰², wherein R¹⁰² is methyl or ethyl.

87. The method of claim 86, wherein R¹⁷ is H, C1-C4 alkyl, or phenyl, optionally substituted with one or more halogen atoms, —NO₂, —NH₂, —COOH, C1-C3 alkyl, C1-C3 carbalkoxy, C1-C3 a alkoxy group, C1-C3 haloalkyl or C1-C3 haloalkoxy.

88. The method of claim 87, wherein R¹⁷ is H, C1-C4 haloalkyl or phenyl optionally substituted with one or more halogen atoms or C1-C3 haloalkyls.

89. The method of claim 88, wherein R² is —H, methyl or ethyl and R⁶ is —H, —OH, methyl, ethyl.

90. The method of claim 89, wherein R¹⁷ is H, trifluoromethyl or phenyl substituted with one or more trifluoromethyls.

91. The method of claim 46, wherein the disorder is systemic lupus, inflammatory bowl disease, psoriasis, Crohn's disease, rheumatoid arthritis, sarcoid, Alzheimer's disease, insulin dependent diabetes mellitus, atherosclerosis, asthma, spinal cord injury, stroke, a chronic inflammatory demyelinating neuropathy, multiple sclerosis, a congenital metabolic disorder, a neuropathy with abnormal myelination, drug-induced demyelination, radiation induced demyelination, a hereditary demyelinating condition, a prion-induced demyelination, encephalitis-induced demyelination.

92. The method of claim 46, wherein one or more additional pharmaceutical agents is co-administered with a compound of formula (I).

93. A method of treating a patient suffering from a cancer, comprising administering to said patient a therapeutically effective amount of any of the compounds of claim 1 of a pharmaceutically acceptable salt thereof, wherein the cancer is selected from the group consisting of colorectal, ovarian, renal, sarcoma, melanoma, head & neck, hepatocellular, thyroid, non-small cell lung cancer, multidrug-resistant leukemia, lymphoma, and multiple myeloma.

94. A method of treating a patient suffering from an acute myeloid leukemia characterized by a FLT3 mutation, comprising administering to the patient a therapeutically effective amount of a compound of claim 1 or a pharmaceutically acceptable salt thereof.
